(19) United States
(12) Patent Application Publication
(10) Pub. No.: US 2023/0265488 A1
(43) Pub. Date: Aug. 24, 2023

(54) COMPOSITIONS AND METHODS FOR USE WITH FIXED SAMPLES

(71) Applicant: 10x Genomics, Inc., Pleasanton, CA (US)

(72) Inventors: Shalini V. Gohil, Pleasanton, CA (US); Luigi Jhon Alvarado Martinez, Pleasanton, CA (US); Albert Dale Kim, Pleasanton, CA (US)

(21) Appl. No.: 18/046,457

(22) Filed: Oct. 13, 2022

Related U.S. Application Data
(63) Continuation of application No. PCT/US2021/027784, filed on Apr. 16, 2021.
(60) Provisional application No. 63/010,768, filed on Apr. 16, 2020.
(60) Provisional application No. 63/132,278, filed on Dec. 30, 2020.

(51) Int. Cl. C12Q 1/6809; C12Q 1/6832; C12Q 1/6874

(52) U.S. Cl. CPC C12Q 1/6809; C12Q 1/6832; C12Q 1/6874

(57) ABSTRACT

Disclosed are compositions, reagents, methods, kits and systems for improving un-fixing or decrosslinking of fixed biological particles, fixed membrane-bound particles, fixed cells, nuclei and/or the biomolecules (e.g., nucleic acids, RNA) of fixed cells or nuclei. Processing of nucleic acids and barcoding nucleic acids are disclosed. Methods for increasing amounts and quality of un-fixed biomolecules (e.g., nucleic acids, RNA) from fixed cells or nuclei are disclosed. Polymers like polyethylene glycol (PEG) increase the amount and quality of ribonucleic acid (RNA) obtained from un-fixed cells or nuclei. Disclosed are methods for using un-fixed cells/nuclei and biomolecules from un-fixed cells/nuclei in various assays. Polymers like PEG increase the efficiency of nucleic acid polymerase reactions (e.g., DNA polymerase, reverse transcriptase) in presence of un-fixing agents. In some examples, PEG increases reverse transcriptase activity, in the presence of proteases, when PEG is included in the reaction.

### CROSS-REFERENCE TO RELATED APPLICATIONS

This application is a continuation of International Application No. PCT/US2021/027784, filed Apr. 16, 2021, which claims priority to U.S. Provisional Application No. 63/010,768, filed Apr. 16, 2020, and to U.S. Provisional Application No. 63/132,278, filed Dec. 30, 2020, each of which is hereby incorporated in its entirety including all tables, figures, and claims.

### BACKGROUND

Detection and quantification of analytes in fixed biological samples, after treatment of the biological particles (e.g., cells or nuclei) in the samples to reverse effects of fixation on biomolecules within the particles (i.e., un-fixing or decrosslinking), is an active area of research and development. The goal is to obtain un-fixed biological particles usable for assays that detect and quantify various analytes/biomolecules in or from the particles. Ideally, the processes of fixing, un-fixing, and subsequent detection of analytes in the un-fixed particles are adapted to rapid, high-throughput systems, such as partitioned single-cell systems, where. As such, not only are outcomes of individual fixation, un-fixing, and detection steps sought to be improved. But, also of interest is rapidly progressing a sample that has undergone one step (e.g., un-fixing or decrosslinking), onto a subsequent step (e.g., enzymatic assays for detecting analytes), and producing a successful result. At least in some examples, current systems and methods for fixation, un-fixing, and/or detection have a relatively low throughput. Therefore, better methods for un-fixing biological particles, for maximizing usable enzymatic substrates and/or templates from the un-fixed particles, and conditions for using the enzymes on these substrates and templates would be useful.

### SUMMARY

Partition-based analysis of biological particles or membrane bound particles (e.g., cells, nuclei, etc.) routinely encounter sample stability challenges. Once removed from a state of relative viability (e.g., removal or dissociation of cells or nuclei from a tissue sample), the risk of sample degradation increases. The use of fixation agents can address sample degradation but then typically requires reversal of the state of fixation in order to access cellular analytes (e.g., ribonucleic acid (RNA) molecules from a single cell or nucleus) for processing in partitions (e.g., single cells or nuclei in droplets in an emulsion or wells). The present disclosure concerns the reversal of this state of fixation in partitions such that cellular analytes are rendered accessible to reagents for processing in the same partitions. Disclosed are compositions, reagents, methods, kits, and systems for un-fixing biological particles (e.g., cells) that have been fixed, to maximize the quantity and quality of analytes detected within the particles (also referred to herein as cellular analytes). Also disclosed are compositions, reagents, methods, and kits for detecting and quantifying analytes from un-fixed cells or nuclei. In one aspect, disclosed are methods for processing a nucleic acid analyte from fixed samples. In some embodiments, the fixed sample contains fixed cells or nuclei. In some embodiments, the methods includes providing a partition that includes: (i) a fixed biological particle or a fixed membrane bound particle that contains a nucleic acid molecule that has a nucleotide sequence, and (ii) a nucleic acid barcode molecule. In some embodiments, the partition further includes an un-fixing, decrosslinking or cleaving agent. In some embodiments, a cleaving agent is a protease. The protease may include Proteinase K, subtilisin A, and/or a cold-active protease, for example. In some embodiments, the un-fixing, decrosslinking or cleaving agent is a catalyst. In other embodiments, the un-fixing/decrosslinking/cleaving agents include both a protease and a catalyst. In some embodiments, the un-fixing/decrosslinking/cleaving agents break bonds generated by fixation agents.

In one other embodiment, the nucleic acid barcode molecule comprises a capture sequence configured to anneal to a nucleic acid molecule of said fixed biological particle or said fixed membrane bound particle. In another embodiment, the capture sequence is a poly-T sequence. In one additional embodiment, the nucleic acid barcode molecule comprises a unique molecular identifier sequence (UMI), or a sequence configured to allow attachment to a flow cell.

In another embodiment, the method comprises partitioning a fixed biological particle or a fixed membrane bound particle comprising a nucleic acid molecule comprising a nucleic acid sequence and (ii) a nucleic acid barcode molecule. In one embodiment, the method comprises subjecting said partition to a condition sufficient to generate a nucleic acid molecule comprising said nucleic acid sequence coupled to said nucleic acid barcode molecule. In other embodiments, the nucleic acid molecule is a cross-linked nucleic acid molecule. In one other embodiment, said cross-linked nucleic acid molecules comprises a ribonucleic acid (RNA) molecule, which can be a messenger RNA (mRNA) molecule. In one embodiment, the subjecting of said partition comprises (i) generating an unlinked nucleic acid molecule from said cross-linked nucleic acid molecule and (ii) allowing said unlinked nucleic acid molecule to couple with said nucleic acid barcode molecule.

In other embodiments, the subjecting of said partition comprises heating said partition. The heating of said partition may comprise heating to a temperature of about 53° C. for about 45 minutes and then optionally at a temperature of about 70° C. for about 15 minutes. In another embodiment, the heating further comprises subjecting said partition to a temperature of about 90° C. for about 10 minutes. In one additional embodiment, the partition is cooled or allowed to cool subsequent to said subjecting of said partition to a condition sufficient to generate a nucleic acid molecule comprising said nucleic acid sequence coupled to said nucleic acid barcode molecule. In one embodiment, said cooling or allowing to cool comprises bringing the partition to a temperature of about 25° C.

In another embodiment, the method comprises releasing said nucleic acid molecule comprising said nucleic acid sequence coupled to said nucleic acid barcode molecule from said partition to generate a released nucleic acid molecule comprising said nucleic acid sequence coupled to said nucleic acid barcode molecule. In one other embodiment, the partition is a droplet or a well. In another embodiment, the releasing comprises breaking said droplet. In one additional embodiment, the partition is cooled or allowed to cool prior to said releasing step. In one embodiment, said cooling or allowing to cool comprises bringing the partition to a temperature of about 25° C.

In other embodiments, the method comprises subjecting said released nucleic acid molecule comprising said nucleic acid sequence coupled to said nucleic acid barcode molecule to a condition sufficient for a nucleic acid extension reaction. In one other embodiment, the nucleic acid extension reaction comprising extension of said nucleic acid barcode molecule, using said nucleic acid sequence as a template, to generate a barcoded nucleic acid molecule. In one embodiment, the extension is an extension of a 3′ end of said nucleic acid barcode molecule.

In one other embodiment, the step of said released nucleic acid molecule to a condition sufficient for a nucleic acid extension reaction i) is performed in a presence of a protease inhibitor, or ii) comprises using a reverse transcriptase to extend said nucleic acid molecule barcode molecule. The reverse transcriptase may comprise RNase activity. In one embodiment, the extension is an extension of a 3′ end of said nucleic acid barcode molecule.

In other embodiments, the method further comprises appending an additional sequence to said barcoded nucleic acid molecule. In one embodiment, the additional sequence is a poly-C sequence. The appending may be performed by a ligase or a polymerase, which can be a reverse transcriptase. In other embodiments, the appending comprises using a splint nucleic acid molecule. In other embodiments, the appending comprises using a primer to anneal to said barcoded nucleic acid molecule.

In one additional embodiment, the partition further comprises a template switching oligonucleotide (TSO). In one other embodiment, the method further comprises i) subjecting said partition to a condition sufficient to hybridize said TSO to said additional sequence, or ii) extending said barcoded nucleic acid molecule to generate an extended barcoded nucleic acid molecule comprising a sequence complementary to said TSO. In one embodiment, the TSO comprises a sequencing primer sequence or complement thereof. In other embodiments, the nucleic acid barcode molecule is coupled to a support, which can be a bead. In one embodiment, the bead is a gel bead. In other embodiments, the nucleic acid barcode molecule is coupled to said support by a labile moiety.

In one other embodiment, the method further comprises sequencing said barcoded nucleic acid molecule or an amplification product thereof. In one embodiment, the method further comprises providing a plurality of partitions. In one additional embodiment, the method comprises, prior to providing a partition or plurality of partitions, partitioning a one or more or a plurality of fixed biological particles or a plurality of fixed membrane bound particles into a plurality of partitions. In one embodiment, the plurality of partitions comprise a plurality of nucleic acid molecules coupled to nucleic acid barcode molecules subsequent to said subjecting said partition to a condition sufficient to generate a nucleic acid molecule comprising said nucleic acid sequence coupled to said nucleic acid barcode molecule and/or prior to releasing said nucleic acid molecule comprising said nucleic acid sequence coupled to said nucleic acid barcode molecule from said partition. In other embodiments, the method comprises, prior to releasing said nucleic acid molecule, pooling said plurality of nucleic acid molecules coupled to said nucleic acid barcode molecules from said plurality of partitions. In additional embodiments, the method further comprises, prior to providing a partition (or partitioning), fixing a biological particle or a membrane bound particle to generate said fixed biological particle or said fixed membrane bound particle. The fixing may comprise use of a fixation agent, which may comprise paraformaldehyde. In another embodiment, the fixed biological particle or said fixed membrane bound particle comprises a cell, virus, or nucleus.

Also disclosed is the discovery that polymers, like polyethylene glycol (PEG), increase the amount of high-quality RNA analytes recovered from un-fixed cells or nuclei. In various examples, PEG increases the amount of RNA obtained from cells or nuclei where the un-fixing agents include proteases and/or other substances. In addition, such polymers, like PEG, were found to increase activity of enzyme reactions performed using un-fixed cells, nuclei and/or the biomolecules obtained from un-fixed cells or nuclei. In some examples, various activities of reverse transcriptase (RT) are increased in the presence of proteases, the proteases which may remain from the un-fixing reactions. The methods and reactions described may be used in partitions, including discrete droplets, which may contain a single cell or nucleus, or the methods/reactions may be used on a larger scale (e.g., in bulk).

Disclosed are compositions containing a reverse transcriptase (RT), a protease and a polyethylene glycol (PEG). The composition may additionally include a nucleic acid template capable of being used by the RT to synthesize a complementary DNA strand, a first primer capable of being used by the RT to initiate first-stand synthesis of the complementary DNA strand, a template switching oligonucleotide, and a second primer capable of being used by the RT to initiate second-strand DNA synthesis. In some examples, the nucleic acid template capable of being used by the RT includes RNA, which may be from un-fixed cells or nuclei, in some examples from un-fixed cells or nuclei in which a protease un-fixing agent has been used.

Disclosed are methods for synthesizing a complementary single-stranded DNA in a composition that includes a reverse transcriptase (RT), a nucleic acid template, a protease, and a polyethylene glycol (PEG). The method may use a composition that additionally includes a nucleic acid template capable of being used by the RT to synthesize a complementary DNA strand, a first primer capable of being used by the RT to initiate first-stand synthesis of the complementary DNA strand, a template switching oligonucleotide, and a second primer capable of being used by the RT to initiate second-strand DNA synthesis. In some examples, the nucleic acid template capable of being used by the RT includes RNA, which may be from un-fixed cells or nuclei, in some examples from un-fixed cells or nuclei in which a protease un-fixing agent has been used.

Disclosed are methods that include: (a) un-fixing cells or nuclei that have been fixed using an un-fixing agent, and (b) synthesizing a complementary single-stranded DNA using a nucleic acid template from the un-fixed cells or nuclei and a reverse transcriptase (RT), in presence of a polyethylene glycol (PEG). The un-fixing agent may have protease activity. The synthesizing step may be performed in presence of protease activity. PEG may be included with the un-fixing agent in the un-fixing step.

Disclosed are methods for un-fixing cells, nuclei or tissues that have been fixed with a fixing agent, where the un-fixing uses an un-fixing agent in presence of a polyethylene glycol (PEG). The un-fixing agent may have protease activity. The un-fixed cells/nuclei/tissues, or biomolecules from the un-fixed cells, nuclei or tissues, may be used in various assays as substrates or templates for enzymatic reactions.

Disclosed are methods for: (a) fixing cells or nuclei with a fixing agent to crosslink biomolecules in the cells or nuclei, and (b) un-fixing the fixed cells or nuclei with an un-fixing agent in the presence of PEG to remove the crosslinks from the biomolecules. The un-fixing agent may have protease activity. In a step (c), a nucleic acid (e.g., RNA) may be isolated from the un-fixed cells or nuclei.

In some examples, any of the methods disclosed herein may be distinguished by having the advantage of high throughput processing of fixed cells or nuclei in partitions (e.g., droplets in an emulsion or wells, such as microwells). As further described herein, the methods of the present disclosure allow for thousands to tens of thousands to hundreds of thousands fixed cells or nuclei to be analyzed on a single cell/nuclei level in individual partitions containing individual cells or nuclei. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

The following U.S. patents and U.S. published patent applications are each incorporated by reference in their entirety into this application:

U.S. Pat. No. 9,644,204 (Ser. No. 14/175,935), issued May 9, 2017 and titled, “Partitioning And Processing Of Analytes And Other Species”;

U.S. Pat. No. 9,975,122 (Ser. No. 14/934,044), issued May 22, 2018 and titled, “Instrument Systems For Integrated Sample Processing”;

U.S. Pat. No. 10,011,872 (Ser. No. 15/720,085), issued Jul. 3, 2018 and titled, “Methods and Systems For Processing Polynucleotides”;

U.S. Pat. No. 10,053,723 (Ser. No. 15/719,459), issued Aug. 21, 2018 and titled, “Capsule Array Devices And Methods Of Use”;

U.S. Pat. No. 10,071,377 (Ser. No. 15/687,856), issued Sep. 11, 2018 and titled, “Fluidic Devices, Systems, And Methods For Encapsulating And Partitioning Reagents, And Applications Of Same”; and

U.S. Pat. No. 10,590,244 (Ser. No. 16/178,430), issued Mar. 17, 2020 and titled, “Compositions, Methods, and Systems For Bead Formation Using Improved Polymers.”

Other references incorporated by reference may be listed throughout the application.

### BRIEF DESCRIPTION OF THE DRAWINGS

In the accompanying drawings, which are incorporated in and constitute a part of the specification, embodiments of the disclosed inventions are illustrated. It will be appreciated that the embodiments illustrated in the drawings are shown for purposes of illustration and not for limitation. It will be appreciated that changes, modifications, and deviations from the embodiments illustrated in the drawings may be made without departing from the spirit and scope of the invention, as disclosed below.

FIG. 1 illustrates an example of a workflow for methods provided herein.

FIGS. 2A-2D shows example schematics for processing nucleic acids.

FIG. 3 shows an example of a microfluidic channel structure for partitioning individual biological particles.

FIG. 4 shows an example of a microfluidic channel structure for delivering barcode carrying beads to droplets.

FIG. 5 shows an example of a microfluidic channel structure for co-partitioning biological particles and reagents.

FIG. 6 shows an example of a microfluidic channel structure for the controlled partitioning of beads into discrete droplets.

FIG. 7 shows an example of a microfluidic channel structure for increased droplet generation throughput.

FIG. 8 shows another example of a microfluidic channel structure for increased droplet generation throughput.

FIG. 9 is a schematic drawing (side view) that illustrates an example of a capture probe attached to a support.

FIG. 10 illustrates an example of a barcode carrying bead.

FIG. 11 illustrates another example of a barcode carrying bead.

FIG. 12 shows an exemplary microwell array schematic.

FIG. 13 shows an exemplary microwell array workflow for processing nucleic acid

FIG. 14 shows a computer system that is programmed or otherwise configured to implement methods provided herein.

FIGS. 15A-15C illustrate example data obtained from single-cell gene expression libraries made from fresh cells and from fixed cells that were un-fixed under various conditions.

FIGS. 16A-16C illustrates example data obtained from single-cell gene expression libraries made from fresh cells and from fixed cells that were un-fixed under various conditions.

FIG. 17 illustrates a tracing of separation of products resulting from an example reverse transcription reaction. The marked regions (1, 2, 3, 4 along the x-axis) were used to calculate transcription efficiency and TSO efficiency, as described in Example 4.

FIG. 18 illustrates example results of reverse transcriptase reaction products obtained using an input template and primers and the reaction products separated by capillary electrophoresis (panel A). Panel B illustrates results when Proteinase K is added to the reaction. Panel C illustrates results when Proteinase K and polyethylene glycol (PEG) 6000 (8% final concentration) are added to the reaction. From left to right in the panels, the peaks represent residual input primer (labeled as “Primer”), first strand cDNA (labeled as “Full Length), and first strand cDNA with the addition of the template-switching sequence (labeled as “TSO”).

FIG. 19 illustrates example results of the amount of reverse transcriptase reaction products obtained using an input template and primers and the reaction products separated by capillary electrophoresis. For each set of conditions, Transcription Efficiency (left bar) and TSO Efficiency (right bar) were calculated as described in Example 4. All reactions contained template, primers, and reverse transcriptase, and were performed for either 45 or 90 minutes. Other additions and conditions for each sample were as follows:

(A) No additions, 45 min.;

(B) Subtilisin A, 45 min.;

(C) Subtilisin A and PEG 6000 (8% final concentration); 45 min.;

(D) Subtilisin A, PEG 6000 (8% final concentration) and protease inhibitor; 45 min.;

(E) No additions, 90 min.;

(F) Proteinase K; 45 min.;

(G) Proteinase K; 90 min.;

(H) Proteinase K and protease inhibitor; 90 min.;

(I) Proteinase K and PEG 6000 (8% final concentration); 45 min.;

(J) Proteinase K, PEG 6000 (8% final concentration) and protease inhibitor; 90 min.; and

(K) Proteinase K and PEG 6000 (4% final concentration); 45 min.

FIG. 20 illustrates example results of the amount of RNA isolated from paraformaldehyde-fixed cells after various decrosslinking/un-fixation conditions. Sample (A) is RNA from fresh, unfixed cell pellets (positive control). All decrosslinking reactions were performed at 53° C. for 45 min. For the decrosslinked samples (B through L), the amount of RNA obtained from the supernatant, after the cells had been decrosslinked and centrifuged, is shown above the black line in some of the bars (samples B, C, G, H, I, J, K and L). For those samples, the amount of RNA obtained from the cell pellet is shown below the black line. For some samples (D, E and F), the amount of RNA in the supernatant was negligible and only RNA from the cell pellet is shown. Additions to each reaction were as follows:

(A) RNA from fresh cells (positive control);

(B) 0.1 mg Proteinase K (PK) per ml;

(C) 0.1 mg PK per ml and 2-amino-5-methylbenzoic acid un-fixer;

(D) 0.1 mg PK per ml and (4-aminopyridin-3-yl)phosphonic acid un-fixer;

(E) 0.1 mg PK per ml, (4-aminopyridin-3-yl)phosphonic acid un-fixer and PEG 6000 (4% final concentration);

(F) 0.1 mg PK per ml, (4-aminopyridin-3-yl)phosphonic acid un-fixer and PEG 6000 (8% final concentration);

(G) 0.1 mg PK per ml, 2-amino-5-methylbenzoic acid and (4-aminopyridin-3-yl)phosphonic acid un-fixers;

(H) 0.1 mg PK per ml, 2-amino-5-methylbenzoic acid un-fixer, (4-aminopyridin-3-yl)phosphonic acid un-fixer and PEG 6000 (4% final concentration);

(I) 0.1 mg PK per ml, 2-amino-5-methylbenzoic acid un-fixer, (4-aminopyridin-3-yl)phosphonic acid un-fixer and PEG 6000 (8% final concentration);

(J) 0.2 mg PK per ml, 2-amino-5-methylbenzoic acid un-fixer and (4-aminopyridin-3-yl)phosphonic acid un-fixer;

(K) 0.2 mg PK per ml, 2-amino-5-methylbenzoic acid 1 un-fixer, (4-aminopyridin-3-yl)phosphonic acid un-fixer and PEG 6000 (4% final concentration); and

(L) 0.2 mg PK per ml, 2-amino-5-methylbenzoic acid un-fixer, (4-aminopyridin-3-yl)phosphonic acid un-fixer and PEG 6000 (8% final concentration).

### DETAILED DESCRIPTION

Provided herein are compositions, reagents, methods, kits, and systems for analyzing nucleic acid molecules from a biological particle that has been fixed or subjected to a cell/nucleus fixative or a cell/nucleus fixation agent. The compositions, reagents, methods, kits, and systems may be used to barcode nucleic acid molecules (e.g., mRNAs) from the biological particle. The compositions, reagents, methods, kits and systems may be able to remove any crosslinks from the nucleic acid molecules such that the nucleic acid molecules can be extracted from the biological particles (e.g., cells or nuclei) and be subjected to downstream reactions, such as barcoding or sequencing. For example, mRNA from a fixed cell or nucleus may be subjected to reactions to allow the mRNA to be barcoded and sequenced. The nucleic acid molecules of a fixed cell or nucleus may be crosslinked to other macromolecules and may be less accessible than nucleic acid molecules of fresh or non-fixed cells or nuclei. Because of a lower accessibility of nucleic acid molecules in fixed cells or nuclei compared to fresh or non-fixed cells/nuclei, it may be more difficult to analyze the nucleic acid molecules from fixed cells or nuclei. The barcoding of the nucleic acid molecules may be used to identify the nucleic acid molecules as originating from a particular biological particle. The compositions, systems, and methods may be used to analyze multiple biological particles (e.g., multiple cells or nuclei) and allow the nucleic acid molecules from the multiple biological particles to be identified as originating from a particular biological particle.

Provided herein is a method for processing a nucleic acid molecule, comprising: a) providing a partition comprising: (i) a fixed biological particle comprising a nucleic acid molecule comprising a nucleic acid sequence and (ii) a nucleic acid barcode molecule; b) subjecting said partition to a condition sufficient to generate a nucleic acid molecule comprising said nucleic acid sequence coupled to said nucleic acid barcode molecule; c) releasing said nucleic acid molecule comprising said nucleic acid sequence coupled to said nucleic acid barcode molecule from said partition to generate a released nucleic acid molecule comprising said nucleic acid sequence coupled to said nucleic acid barcode molecule; and d) subjecting said released nucleic acid molecule comprising said nucleic acid sequence coupled to said nucleic acid barcode molecule to a condition sufficient to extend said nucleic acid barcode molecule, using said nucleic acid sequence as a template, to generate a barcoded nucleic acid molecule. In one embodiment, the condition sufficient is a condition sufficient to extend a 3′ end of said nucleic acid barcode molecule. In one embodiment, the fixed biological particle comprises a membrane or is a fixed membrane bound biological particle. The fixed biological particle or fixed membrane bound particle may be a fixed cell or nucleus.

In some examples, polymers, like polyethylene glycol (PEG), may increase the amount and/or quality of nucleic acids recovered from fixed cells or nuclei that have been un-fixed. In some examples, polymers may increase the activity of enzymes in various reactions performed using un-fixed cells or nuclei. The methods and reactions described may be used in partitions, including discrete droplets, which may contain a single cell or nucleus, or the methods/reactions may be used on a larger scale (e.g., in bulk).

### Definitions

The terms “a,” “an,” and “the,” as used herein, generally refers to singular and plural references unless the context clearly dictates otherwise.

Herein, “absence of” means without.

Herein, “activity,” generally when referring to an enzyme, means the amount of, or extent to which, an enzyme catalyzes a reaction.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

Herein, “agent” means a substance to which a particular activity can be attributed.

Herein, “amplification product” refers to molecules that result from reproduction or copying of another molecule. Generally, the molecules copied or reproduced are nucleic acid molecules, specifically DNA or RNA molecules. In some examples, the molecule reproduced or copied may be used as a template for the produced molecules. In some examples, an analyte captured by the capture domain of an oligonucleotide may be used as a template to produce an amplification product. In some examples, an mRNA captured by the capture domain of an oligonucleotide may be used as a template to produce a cDNA amplification product. Various enzymes (e.g., reverse transcriptase) may be used for this process. The cDNA amplification product may in turn act as a template for amplification that may also be called amplification products. Various enzymes (e.g., Taq polymerase) may be used for this process.

Herein, “analyte” refers to a substance whose chemical constituents are being identified and/or measured. Generally, this application refers to analytes from and/or produced by cells or nuclei. Any or all molecules or substance from or produced by a cell or nucleus may be referred to herein as analytes. Chemically, cellular analytes may include proteins, polypeptides, peptides, saccharides, polysaccharides, lipids, nucleic acids, and other biomolecules.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Herein, “average” refers to a mean.

Herein, “barcode” generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

Herein, “barcoded nucleic acid molecule” generally refers to a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence (e.g., targeted by a primer sequence) or a non-targeted sequence. For example, in the methods, compositions, kits, and systems described herein, hybridization and reverse transcription of the nucleic acid molecule (e.g., a messenger RNA (mRNA) molecule) of a cell or nucleus with a nucleic acid barcode molecule (e.g., a nucleic acid barcode molecule containing a barcode sequence and a nucleic acid primer sequence complementary to a nucleic acid sequence of the mRNA molecule) results in a barcoded nucleic acid molecule that has a sequence corresponding to the nucleic acid sequence of the mRNA and the barcode sequence (or a reverse complement thereof). A barcoded nucleic acid molecule may be a nucleic acid product. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the mRNA.

Herein, “base-paired” generally refers to the situation where two complementary nucleic acids have formed hydrogen bonds between complementary nucleotides in the different strands. Two such nucleic acid strands may be referred to as hybridized to one another.

The term “bead” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold, or silver) covered with a coating comprising one or more polymers. Such coating may be disruptable or dissolvable.

The term “biological particle,” as used herein, generally refers to a discrete biological system derived from a biological sample. The biological particle may be a macromolecule. The biological particle may be a small molecule. The biological particle may be a virus. The biological particle may be a cell, a nucleus or a derivative of a cell. The biological particle may be an organelle. The biological particle may be an organelle from a cell. Examples of an organelle from a cell include, without limitation, a nucleus, endoplasmic reticulum, a ribosome, a Golgi apparatus, an endoplasmic reticulum, a chloroplast, an endocytic vesicle, an exocytic vesicle, a vacuole, and a lysosome. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell but may not include other constituents of the cell. An example of such constituents is a nucleus or an organelle. A cell may be a live cell. The live cell may be capable of being cultured, for example, being cultured when enclosed in a gel or polymer matrix or cultured when comprising a gel or polymer matrix.

Herein, “biomolecule” or “biological molecule” generally refers to substances produced by cells or nuclei. Example groups of biomolecules include proteins, nucleic acids, carbohydrates, and lipids.

Herein, “capable” means having the ability or quality to do something.

Herein, “capture” generally refers to the capability of a first substance to interact with and/or bind a second substance where, for example, the second substance is part of a population of other substances. An analyte may be captured. In some examples, capture refers to identification of a target nucleic acid molecule (e.g., an RNA) by its hybridization to a capture probe, and/or amplification of a target nucleic acid molecule or a nucleic acid probe hybridized to it (e.g., an RNA or a probe hybridized to the RNA) using, for example polymerase chain reaction (PCR) and/or nucleic acid extension of a target nucleic acid molecule or a capture probe hybridized to it using, for example reverse transcription reactions.

Herein, “capture probe” refers to a molecule (e.g., an oligonucleotide) that contains a capture domain.

Herein, “capture domain” or “capture sequence” means a part of a molecule that is capable of binding or capturing a substance. An analyte capture domain may be capable of capturing analytes that may include proteins, polypeptides, peptides, saccharides, polysaccharides, lipids, nucleic acids, and other biomolecules. In some examples, the analyte capture domain may be a nucleotide sequence capable of hybridizing to an analyte that contains a complementary nucleotide sequence. Herein, “nucleotide capture sequence” refers to a first nucleotide sequence that is capable of capturing (e.g., by hybridizing to) a second nucleotide sequence. In some examples, an analyte capture domain may contain modified nucleotides.

Herein, “catalyst” refers to a substance that increases a rate of a reaction, generally without undergoing changes itself. Herein, “catalyst” may refer to a substance that un-fixes/decrosslinks nucleic acid molecules. Example catalysts are indicated in Table 1 herein.

Herein, “cleaving agent” is the same as un-fixing and de-crosslinking agents (see definition of “un-fix).

Herein, “cold-active protease” or “CAP” refers to proteases that have protease active at temperatures less than about 37° C. In some examples, CAP have protease activity at temperatures less than about 25° C. In some examples, CAP may have protease activity at temperatures of about 15-25° C. or about 5-15° C. In some examples, a CAP may be ArcticZymes Proteinase (ArcticZymes Technologies ASA, Tromso, Norway), alcalase, alkaline proteinase, bacillopeptidase A, bacillopeptidase B, bioprase, colistinase, esperase, genenase, kazusase, maxatase, proteinase K, protease S, savinase, Serratia peptidase (i.e., peptidase derived from Serratia sp.), subtilisin A, subtilisin B, subtilisin BL, subtilisin E, subtilisin J, subtilisin S, subtilisin S41, thermoase, trypsin, and a combination thereof. Additional CAP compositions, methods, and kits are disclosed in PCT/US2021/026592, which is incorporated herein by reference in its entirety.

Herein, “complementary,” in the context of one sequence of nucleic acids being complementary to another sequence, refers to the ability of the two strands to form hydrogen bonds between the two strands, along their full length. A complementary strand of nucleic acids is generally made using a template nucleic acid strand.

Herein, “contribute to,” in reference to a substance causing an activity or result, generally refers to how much of the activity or result can be attributed to the specific sub stance.

Herein, “couple” means to join or connect. Herein, “hybridization” is a type of coupling.

Herein, “crosslinking” means connecting or attaching two or more separate substances to each other. The connecting or attaching is due to formation of crosslinks. In some examples, crosslinking refers to formation of chemical bonds between two or more atoms in a molecule or in different molecules. In some examples, nucleic acid molecules may be crosslinked. A nucleic acid molecule that is not crosslinked maybe said to be “unlinked.”

Herein, “deoxyribonucleic acid” or “DNA” refers to a nucleic acid formed from polymerization of deoxyribonucleotides.

Herein, “deoxyribonucleotide” or “dNTP” means a nucleotide that contains deoxyribose and is a constituent of DNA.

Herein, “derived from,” when referring to a second substance or molecule that is derived from a first substance or molecule, refers to a second substance that is different than, but related to, the first substance. In an example where the amino acid sequence of a first enzyme is changed by substituting one amino acid with another to yield a second enzyme, the second enzyme can be said to be derived from the first enzyme.

Herein, “DNA polymerase” refers to an enzyme that synthesizes DNA from deoxyribonucleotides.

Herein, “droplet” refers to a small portion of a liquid, generally round or pear-shaped.

Herein, “enzyme” refers to molecules, generally proteins, that catalyze biochemical reactions.

Herein, “equivalent” means the same as.

Herein, “extend” generally refers to synthesis of a nucleic acid strand by extending a primer and using a template. Extension may also refer to extension of a nucleic strand using terminal transferase activity of a reverse transcriptase, for example.

Herein, “first-strand synthesis” refers to a complementary DNA strand made using a nucleic acid template, generally an RNA template, by a reverse transcriptase enzyme.

Herein, “final concentration” generally refers to the concentration of a substance in a mixture in which no additional components will be added.

Herein, “fix” refers to formation of covalent bonds, such as crosslinks, between biomolecules or within molecules. The process of fixing, cells or nuclei for example, is called “fixation.” The agent that causes fixation is generally referred to as a “fixative” or “fixing agent.” “Fixed cells” or “fixed tissues” or “fixed nuclei” refers to cells, nuclei or tissues that have been in contact with a fixative under conditions sufficient to allow or result in the formation of intra- and inter-molecular covalent crosslinks between biomolecules in the biological sample.

Herein, “flow cell” refers to a slide, generally a glass slide, that contains fluidic channels. In some examples, the term “flow cell” refers to an Illumina® flow cell or equivalent.

Herein, “gel” means a semisolid colloidal suspension of a solid dispersed in a liquid. A gel is a type of medium. Types of gel may include agar, agarose, hydrogel, polyacrylamide, and the like.

The term “genome” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all these together may constitute a human genome.

Herein, “hybridize” refers to a nucleotide sequence of a single-stranded nucleic acid molecule forming a complex with a nucleic acid molecule having a complementary nucleotide sequence. Generally, the complex forms through hydrogen bonding between complementary nucleotide bases in separate nucleic acid molecules. “Anneal” is another term for hybridize.

Herein, “hybridizing nucleotide sequence” refers to a nucleotide sequence, within an oligonucleotide for example, that is capable of hybridizing with a complementary nucleotide sequence in a target nucleic acid molecule present on or within a cell from a tissue sample (e.g., cellular RNA). When a hybridizing nucleotide sequence is of such a length that it hybridizes with a complementary, either fully or partially, nucleotide sequence that is unique to a target nucleic acid molecule(s) (e.g., cellular RNA or family of RNAs), the hybridizing nucleotide sequence may be said to hybridize to the same target nucleic acid molecule (e.g., the same RNA).

Herein, “initiate” or “initiation” means to start.

Herein, “ligase” refers to an enzyme and/or activity that joins breaks in the phosphodiester backbone of DNA. Ligases generally form phosphodiester bonds.

Herein, “longer than,” when referring to a first nucleic acid molecule being longer than a second nucleic acid molecule, means that the first nucleic acid contains more nucleotides than the second.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

Herein, “membrane bound particle” may refer to a cell, a nucleus, a cellular organelle, or a non-cellular membrane-bound particle (e.g., liposome).

Herein, “mixture” refers to a combination of multiple chemical substances that have not reacted with one another.

Herein, “molecular mass” refers to the sum of the atomic masses of the atoms in a molecule.

The term “molecular tag” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Herein, “nucleic acid” refers to linear macromolecules formed from polymerization of units called nucleotides.

Herein, a “nucleic acid product” refers to a nucleic acid produced using a target nucleic acid molecule (e.g., an RNA) as a template, and derivatives thereof. In some examples, a nucleic acid probe (e.g., an RNA capturing probe) may act as a primer for a nucleic acid extension reaction (e.g., a reverse transcription reaction or a polymerase chain reaction) that extends (or amplifies) a nucleotide sequence of the target nucleic acid molecule, thus generating nucleic acid products based on the target nucleic acid molecule or the nucleic acid probe.

Herein, “nucleotide sequence” or “nucleic acid sequence” refers to a linear progression of nucleotide bases within a nucleic acid molecule (e.g., oligonucleotide).

Herein, “oligomer” refers to a polymer with relatively few repeating units.

Herein, “oligonucleotide” means a linear polymer of nucleotides, in some examples 2′-deoxyribonucleotides. Oligonucleotides are single stranded. Oligonucleotides can be of various lengths. Oligonucleotides can include modified nucleotides as known in the art.

Herein, “partial” means less than all or less than complete.

The term “partition” as used herein, generally, refers to a space or volume that may be suitable to contain one or more species or conduct one or more reactions. A partition may be a physical compartment, such as a droplet or well (e.g., microwell). The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

Herein, “PEGylate” or “pegylate” means that polyethylene glycol (PEG) and/or an amalgamation thereof, is covalently or noncovalently attached to something else, like a biomolecule, for example. “PEGylation” refers to the process by which PEG is attached. Herein, adding a solution of PEG to a reaction solution that contains reverse transcriptase, for example, is not PEGylation of the reverse transcriptase (i.e., the PEG is not attached to the reverse transcriptase). However, the methods disclosed in this application include situations where a solution of PEG is added to an enzymatic reaction and is not attached, as well as situations where PEG is attached to an enzyme in the reaction.

Herein, “polyethylene glycol” refers to a polyether compound with the chemical formula H—(O—CH₂—CH₂)ₙ—OH.

Herein, “performed” means carried out or accomplished.

Herein, “polymer” refers to a substance having many repeating units.

Herein, “polymerase” generally refers to enzymes and/or activities that catalyze formation of nucleic acids from precursor substances.

Herein, “presence of” generally refers to being in the same area or space. In one example, when an enzyme is stated to catalyze a reaction in the “presence of” another substance, the enzyme and the substance will be part of the same solution and, generally, in contact with one another.

Herein, “primer” means a single-stranded nucleic acid sequence that provides a starting point for DNA synthesis. Generally, a primer has a nucleotide sequence that is complementary to a template, and has an available 3′-hydroxyl group to which a transcriptase or polymerase can add additional nucleotides complementary to corresponding nucleotides in the template, to synthesize a nucleic acid strand in the 3′ to 5′ direction.

Herein, “protease” refers to molecules, generally enzymes, and/or activities that can break down proteins by hydrolysis, generally into peptides and/or amino acids.

Herein, “Proteinase K” refers to a specific protease.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection, or identification.

Herein, “release” means to let go or escape.

Herein, “reverse transcriptase” refers to an enzyme and/or activity that can synthesize single-stranded DNA using an RNA template.

Herein, “ribonucleic acid” or “RNA” refers to a nucleic acid formed from polymerization of ribonucleotides.

Herein, “ribonuclease” or “RNase” refers to an enzyme and/or an activity that catalyzes degradation of RNA into smaller components.

Herein, “RNA polymerase” refers to an enzyme and/or an activity that can synthesize single-stranded RNA using a DNA template.

The term “sample” or “biological sample” generally refers to a biological sample of a subject. The biological sample may comprise any number of macromolecules, for example, cellular macromolecules. The sample may be a cell or nuclei sample. The sample may be a cell line or cell culture sample. The sample can include one or more cells/nuclei. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool, and tears. In some examples, a biological sample may be a suspension of cells or nuclei and the contents of the suspension cells or nuclei.

Herein, “second strand synthesis” refers to a complementary DNA strand made using, as template, a DNA strand made by first-strand synthesis.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

Herein, “strand” generally refers to a nucleic acid. Often, “strand” is used in the context of a “single-stranded” nucleic acid or a “double-stranded” nucleic acid. Two nucleic acid strands may or may not be complementary

The term “subject” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

Herein, “subsequent to” means after.

Herein, “substrate” refers to the molecule on which an enzyme acts.

Herein, “support,” when used as a noun, refers to something that serves, for example, as a foundation for another thing. In some examples, the support may be larger, more easily worked with, or more easily tracked or visualized than the thing being supported. A support may be a solid support. In some instances, a support may be dissolvable, disruptable, and/or degradable. In some cases, a support may not be degradable. A support may comprise a glass, plastic, metal, and/or other substances. In some cases, the support can be rigid. In other cases, the support may be flexible and/or compressible. In some examples, a support may be referred to as a “substrate.”

Herein, “surface” means the outside part or upper layer of something. Herein, a “surface” of an array generally refers to a surface of a support or substrate that has oligonucleotides attached.

Herein, “synthesize” generally refers to make something chemically.

Herein, “subtilisin” refers to a specific group of proteases. In some examples, a subtilisin is subtilisin A.

Herein, “template” refers to one single-stranded nucleic acid acting as a “template” for synthesis of another complementary single-stranded nucleic acid. For example, RNA can act as a template for synthesis of a complementary DNA strand synthesized using reverse transcriptase. A single-stranded DNA can act as a template for synthesis of a complementary DNA strand, most often by a DNA polymerase.

Herein, “template switching” or “TS” refers to a process that takes advantage of an activity of some reverse transcriptases that, upon reaching the end (5′ end) of an RNA template during first-strand DNA synthesis, the reverse transcriptase adds multiple nucleotides (generally deoxycytidines) to the 3′ end of the first-strand DNA using terminal transferase activity of the reverse transcriptase (i.e., without use of a template). By adding to the reaction, a DNA oligomer or primer that has a stretch of riboguanosines at its 3′ end (called a template switching oligo, TS oligo or TSO), the riboguanosines will base pair with the stretch of deoxycytidines that is part of the 3′ end of the first-strand DNA. Using the TS oligo as a template, synthesis of the first-strand DNA may then be extended by the reverse transcriptase to the 5′ end of the TS oligo, which is acting as a template for the reverse transcriptase. In this extension, the reverse transcriptase “switches” templates, from the original RNA template to the TS oligo. Second-strand synthesis may then initiate at the 5′ end of the riboguanosine stretch of the TS oligo.

Herein, “terminal transferase” refers to an enzyme activity that adds deoxynucleotides to the 3′ hydroxyl end of a DNA molecule in a template-independent manner.

Herein, “template switching oligo,” “TS oligo” or “TSO” generally refers to a DNA oligomer that has a stretch of riboguanosines at its 3′ end. TSOs may be used in reverse transcription reactions to extend the length of single-stranded DNA that is made during first-strand synthesis. Generally, the second-strand synthesis product is also longer when a TSO is used, compared to when TSOs are not used.

Herein, “un-fix” or “decrosslink” refers to breaking or reversing the formation of covalent bonds in biomolecules formed by fixatives. “Un-fixing agents” (or “decrosslinking agents”) refer to the substances that cause the un-fixing. In some examples, un-fixing or decrosslinking may occur when a reversible fixing agent is used, and when the fixation caused by these agents is reversed.

The terms “un-fixing agent,” “de-crosslinking agent” or “cleaving agent,” as used herein, refers to a compound or composition that reverses fixation and/or removes the crosslinks within or between biomolecules in a sample caused by previous use of a fixation reagent. In some embodiments, these agents are compounds that act catalytically in removing crosslinks in a fixed sample. Herein, an un-fixing/decrosslinking agent may be a protease. Herein, an un-fixing/decrosslinking agent may be a catalyst. Other types of un-fixing/decrosslinking agents may be used.

Herein, “un-fixed” refers to the processed condition of a cell or nucleus, a plurality of cells/nuclei, a tissue sample or any other biological sample that is characterized by a prior state of fixation followed by a reversal of the prior state of fixation. For instance, an un-fixed cell or nucleus may also be referred to as a “previously fixed” cell or nucleus. In one embodiment, an un-fixed cell or nucleus is characterized by broken or reversed covalent bonds in the biomolecules of the cell(s), nuclei or sample, where such covalent bonds were previously formed by treatment with a fixation agent (e.g., paraformaldehyde or PFA).

Herein, “unique molecular identifier” or “UMI” generally refers to an identifier of a particular analyte captured by a capture probe.

Herein, “unlinked” generally refers to a crosslinked molecule that is no longer crosslinked.

Herein, “variant” generally refers to proteins that are modified (e.g., by amino acid substitutions, deletions, insertions, and the like) as compared to the parent or starting protein.

Herein, “well” generally refers to a depression, for example in a multiwell dish, that can hold liquid and may be used for various assays.

### Fixation

Biological samples are unstable. When a biological sample is removed from its viable niche or environment, physical decomposition begins immediately. The degree of decomposition is determined by a number of factors including time, solution buffering conditions, temperature, source (e.g. certain tissues and cells/nuclei a have higher levels of endogenous RNase activity), biological stress (e.g. enzymatic tissue dissociation can activate stress response genes), and physical manipulation (e.g. pipetting, centrifuging). The degradation includes nucleic acid molecules (e.g., RNA), proteins, as well as higher-order 3D structure of molecular complexes, whole cells, tissues, organs, and organisms. The instability of biological samples is a significant obstacle for their use in a variety of assays, including droplet-based genomic assays. Sample degradation greatly limits the ability to use such assays accurately and reproducibly with a wide range of available biological samples.

Example methods for preserving biological sample integrity, and limiting decomposition include cryopreservation, dehydration (e.g., methanol), high salt storage (e.g., using RNAssist, or RNAlater®), and treatment with chemical fixing agents. Chemical fixing agents typically create covalent crosslinks in the biomolecules of the sample (e.g., paraformaldehyde). These techniques for stabilizing biological samples can be used alone or in combination, and each can be reversed to various extents using various un-fixing treatments.

The term “fixed” as used herein regarding biological samples, and the tissues, cells, nuclei and molecules contained in the samples, refers to the state of being preserved from decay and/or degradation. “Fixation” refers to a process that results in a fixed sample, and can include contacting the biomolecules within a biological sample with a fixative (or fixing agent) for some amount of time, whereby the fixative results in covalent bonding interactions such as crosslinks between and within biomolecules in the sample. A “fixed biological sample” refers to a biological sample that has been contacted with a fixation reagent. For example, a formaldehyde-fixed biological sample has been contacted with the fixing agent formaldehyde.

The formation of crosslinks in biomolecules (e.g., proteins, RNA, DNA) due to fixation greatly reduces the ability to detect (e.g., bind to, amplify, sequence, hybridize to) the biomolecules in standard assay methods. Common techniques to remove the crosslinks induced by fixative reagents (e.g., heat, acid) can cause further damage to the biomolecules (e.g., loss of bases, chain hydrolysis, cleavage, denaturation, etc.). Further description of the consequences of fixation of tissue samples and the benefits of removing adducts and/or crosslinks are described in U.S. Pat. No. 8,288,122, which is hereby incorporated by reference in its entirety. For example, the widely used fixing agent, paraformaldehyde or PFA, fixes tissue samples by catalyzing crosslink formation between basic amino acids in proteins, such as lysine and glutamine. Both intra-molecular and inter-molecular crosslinks can form in the protein. These crosslinks can preserve protein secondary structure and eliminate enzymatic activity of proteins in the preserved tissue sample.

The present invention provides methods, composition, kits, and systems for treating fixed biological samples to process cellular analytes. The cellular analytes may be affected by the fixation process. Un-fixing may be used to prepare the analytes for processing. Cellular analytes that are suitable for use with the present invention include, without limitation, intracellular and extracellular analytes. The cellular analyte may be a protein, a metabolite, a metabolic byproduct, an antibody or antibody fragment, an enzyme, an antigen, a carbohydrate, a lipid, a macromolecule, or a combination thereof (e.g., proteoglycan) or another biomolecule. The cellular analyte may be a nucleic acid molecule. The cellular analyte may be a deoxyribonucleic acid (DNA) molecule or a ribonucleic acid (RNA) molecule. The DNA molecule may be a genomic DNA molecule. The cellular analyte may comprise coding or non-coding RNA. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA.

In some instances, the cellular analyte is associated with an intermediary entity, wherein the intermediary entity is analyzed to provide information about the cellular analyte and/or the intermediary entity itself. For instance, an intermediary entity (e.g., an antibody) may be bound to an extracellular analyte (e.g., a cell surface receptor), where the intermediary entity is processed to provide information about the intermediary entity, the extracellular analyte, or both. In one embodiment, the intermediary entity comprises an identifier (e.g., a barcode molecule) that can be used to generate barcode molecules (e.g., droplet-based barcoding) as further described herein.

In some examples, nucleic acid molecules (and biological particles or membrane bound particles) may be crosslinked by one or more fixation reagents. The crosslinking may link multiple molecules within a biological particle. Multiple biological particles may be subjected to a crosslinking reaction to generate a plurality of fixed biological particles or fixed membrane bound particles. For example, the nucleic acid molecules may be derived from a fixed sample or a fixed tissue. Fixation of a biological particle, a membrane bound particle, a cellular constituent or a tissue comprising a plurality of cells, may comprise application of a chemical species or chemical stimulus, such as a fixation agent. The fixation agent may comprise paraformaldehyde. In some cases, a fixation agent may be selected from the group consisting of paraformaldehyde, glutaraldehyde disuccinimidyl suberate (DSS), dimethylsuberimidate (DMS), formalin, and dimethyladipimidate (DMA), dithio-bis(-succinimidyl propionate) (DSP), disuccinimidyl tartrate (DST), and ethylene glycol bis(succinimidyl succinate) (EGS). Other reagents and methods can be used to fix biological samples (e.g., cells or nuclei) including, without limitation, the fixing agents described in PCT/US2020/066705, which is incorporated by reference herein in its entirety.

Fixation may also affect other features of a cell, nucleus or cellular constituent. For example, fixation may result in a change in the porosity of a membrane (cell or nucleus) or wall of a cell; reorganization of components of the cell or nucleus; a change in cell/nucleus fluidity or rigidity; or other changes. Changes to a characteristic or set of characteristics of a cell, nucleus or cellular constituents (e.g., incurred upon interaction with one or more fixation agents) may be at least partially reversible (e.g., via rehydration or de-crosslinking). Alternatively, changes to a characteristic or set of characteristics of a cell or cellular constituents (e.g., incurred upon interaction with one or more fixation agents) may be substantially irreversible.

Generally, contact of a biological sample (e.g., a cell or nucleus) with a fixing agent (e.g., paraformaldehyde or PFA) under appropriate conditions results in the formation of intra- and inter-molecular covalent crosslinks between biomolecules in the biological sample. In some cases, the fixation reagent, formaldehyde, is known to result in covalent aminal crosslinks in RNA, DNA, and/or protein molecules. Examples of fixing agents include but are not limited to aldehyde fixatives (e.g., formaldehyde, also commonly referred to as “paraformaldehyde,” “PFA,” and “formalin”; glutaraldehyde; etc.), imidoesters, NHS (N-Hydroxysuccinimide) esters, and the like.

In some embodiments, the fixative or fixing agent useful in the methods of the present disclosure is formaldehyde. The term “formaldehyde” when used in the context of a fixative also may refer to “paraformaldehyde” (or “PFA”) and “formalin”, both of which are terms with specific meanings related to the formaldehyde composition (e.g., formalin is a mixture of formaldehyde and methanol). Thus, a formaldehyde-fixed biological sample may also be referred to as formalin-fixed or PFA-fixed. Protocols and methods for the use of formaldehyde as a fixation reagent to prepare fixed biological samples are well known in the art and can be used in the methods and compositions of the present disclosure. For example, suitable ranges of formaldehyde concentrations for use in preparing a fixed biological sample is 0.1 to 10%, 1-8%, 1-4%, 1-2%, 3-5%, or 3.5-4.5%. In some embodiments of the present disclosure, the biological sample is fixed using a final concentration of 1% formaldehyde, 4% formaldehyde, or 10% formaldehyde. Typically, the formaldehyde is diluted from a more concentrated stock solution—e.g., a 35%, 25%, 15%, 10%, 5% PFA stock solution.

The amount of time a biological sample is contacted with a fixative to provide a fixed biological sample depends on the temperature, the nature of the sample, and the fixative used. For example, a biological sample can be contacted by a fixation agent for 72 or less hours (e.g., 48 or less hours, 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes). Various temperatures may be used during a fixation process.

It is contemplated that more than one fixation reagent may be used in combination in preparing a fixed biological sample. For example, in some cases biomolecules (e.g., biological samples such as tissue specimens) are contacted with a fixing agent containing both formaldehyde and glutaraldehyde, and thus the contacted biomolecules can include fixation crosslinks resulting both from formaldehyde induced fixation and glutaraldehyde induced fixation. Typically, a suitable concentration of glutaraldehyde for use as a fixation reagent is 0.1 to 1%.

In some examples, reversible fixation agents may be used. In some examples, such fixation agents may be bis-imidazole-carboxylate based compounds.

The biological particle, membrane bound particle, or nucleic acid molecules may be subjected to a fixation process at any useful point in time. For example, cells, nuclei and/or cellular constituents of a sample may be subjected to a fixation process involving one or more fixation agents (e.g., as described herein) prior to commencement of any subsequent processing, such as for storage. Cells, nuclei and/or cellular constituents, such as cells/nuclei and/or cellular constituents of a tissue sample, subjected to a fixation process prior to storage may be stored in an aqueous solution, optionally in combination one or more preserving agents configured to preserve morphology, size, or other features of the cells, nuclei and/or cellular components. Fixed cells or nuclei and/or cellular constituents may be stored below room temperature, such as in a freezer. Alternatively, cells or nuclei and/or cellular constituents of a sample may be subjected to a fixation process involving one or more fixation agents subsequent to one or more other processes, such as filtration, centrifugation, agitation, selective precipitation, purification, permeabilization, isolation, heating, etc. For example, cells or nuclei and/or cellular constituents of a given type from a sample may be subjected to a fixation process following a separation and/or enrichment procedure (e.g., as described herein). In an example, a sample comprising a plurality of cells or nuclei including a plurality of cells or nuclei of a given type may be subjected to a positive separation process to provide a sample enriched in the plurality of cells or nuclei of the given type. The enriched sample may then be subjected to a fixation process involving one or more fixation agents (e.g., as described herein) to provide an enriched sample comprising a plurality of fixed cells or nuclei. A fixation process may be performed in a bulk solution. In some cases, fixed biological particles or fixed membrane bound particles (e.g., fixed cells, nuclei and/or cellular constituents) may be partitioned amongst a plurality of partitions (e.g., droplets or wells) and subjected to processing as described elsewhere herein. In some cases, fixed biological particles or fixed membrane bound particles may undergo additional processing, such as partial or complete reversal of a fixation process by, for example, rehydration or de-crosslinking, prior to partitioning and any subsequent processing. In some cases, fixed biological particles or fixed membrane bound particles may undergo partial or complete reversal of a fixation process within a plurality of partitions (e.g., prior to or concurrent with additional processing described elsewhere herein).

### Un-Fixing/Decrosslinking

The ability to use a fixed biological sample in various assays, including partition-based genomic assays, requires rapid and efficient un-fixing of the sample to obtain the relevant genomic assay information before degradation of the sample occurs. Ideally, the genomic assay data obtained from an un-fixed biological sample should be identical to that obtained from a fresh sample that has not been fixed, or resemble a sample obtained from its natural environment as closely as possible.

Conditions for reversing the effects of fixing a biological sample are known in the art, however, these conditions tend to be harsh. See e.g., WO2001/46402; US2005/0014203A1, and US2009/0202998A1, each of which is hereby incorporated by reference in its entirety. For example, un-fixing treatment of PFA-treated tissue samples includes heating to 60-70° C. in Tris buffer for several hours, and yet typically results in removal of only a fraction of the fixative-induced crosslinks. Furthermore, the harsh un-fixing treatment conditions can result in permanent damage to biomolecules, particularly nucleic acids, in the sample. Recently, less harsh un-fixing techniques and conditions have been proposed that utilize compounds capable of chemically reversing the crosslinks resulting from fixation. See e.g., Karmakar et al., “Organocatalytic removal of formaldehyde adducts from RNA and DNA bases,” Nature Chemistry, 7: 752-758 (2015); US 2017/0283860A1; and US 2019/0135774A1, each of which is hereby incorporated by reference in its entirety.

The terms “un-fixing agent” (or “decrosslinking agent” or cleaving agent”) as used herein, refers to a compound or composition that reverses fixation and/or removes the crosslinks within or between biomolecules in a sample caused by previous use of a fixation reagent. In some embodiments, un-fixing agents are compounds that act catalytically to remove or break crosslinks in a fixed sample.

In various aspects described herein, nucleic acid molecules from fixed biological particles (e.g., fixed membrane bound particles) are subjected to reactions to generate barcoded nucleic acid molecules. The nucleic acid molecules may comprise DNA. The nucleic acid molecules may be derived from genomic DNA or comprise genomic DNA. The nucleic acid molecule may comprise RNA. For example, the nucleic acid molecule may be an mRNA molecule. The nucleic acid molecules may be crosslinked nucleic acid molecules. The biological particle or membrane bound particle may be cell, nucleus, virus, or nucleus.

The nucleic acid molecules may be subjected to reactions to remove the crosslinks or cleave macromolecules such that the nucleic acid molecules can be extracted from the fixed biological particles or fixed membrane bound particles. The reactions may comprise a cleaving agent. For example, the cleaving agent may be an enzyme to cleave bonds. For example, the enzyme may be a protease. The protease may be Proteinase K, subtilisin A, a cold-active protease, and the like. The protease may cleave at a particular amino acid residue. The protease may non-discriminatively cleave proteins in the fixed membrane particle.

The cleaving agent may be a catalyst (e.g., Table 1). For example, the catalyst may catalyze a bond cleaving event such that a nucleic acid may be able to be extracted from the fixed membrane bound particle. The cleaving agent may cleave a bond generated by a fixation agent. For example, the fixation agent may form a particular bond and generate a fixed particle and cleaving agent may cleave the same bond generated by the fixation agent. Alternatively, the cleaving agent may cleave another bond, which is not generated by the fixation agent. For example, the fixation agent may crosslink a nucleic acid to a polypeptide, and the cleaving agent may cleave a bond in the polypeptide such as a peptide bond.

In some examples, the un-fixing agents are proteases. Various proteases may be used. Proteases used in the present disclosure may include serine proteases, cysteine proteases, threonine proteases, aspartic proteases, glutamic proteases, metalloproteases, asparagine peptide lyases, and others. Proteases used in the compositions, methods, reagents, and kits disclosed here may come from many different organisms. The proteases may be variants of or derived from other proteases.

In some examples, the protease is Proteinase K. In some examples, the protease is a subtilisin. In some examples, the protease is subtilisin A. In some examples, the protease may be a cold-active protease. Combinations of proteases may be used.

In some examples, the un-fixing agents may include substances such as 2-amino-5-methylbenzoic acid, 2-amino-5-nitrobenzoic acid, (2-amino-5-methylphenyl)phosphonic acid, 2-amino-5-methylbenzenesulfonic acid, 2,5-diaminobenzenesulfonic acid, 2-amino-3,5-dimethylbenzenesulfonic acid, (2-amino-5-nitrophenyl)phosphonic acid, (4-aminopyridin-3-yl)phosphonic acid, and (2-amino-5-{[2-(2-poly-ethoxy)ethyl]carbamoyl}phenyl)phosphonic acid. In some embodiments, un-fixing agents are compounds that act catalytically in removing crosslinks in a fixed sample.

In some examples, the un-fixing agents may include substances having the chemical formulas, below:

TABLE 1
Un-fixing/Decrosslinking Agents (Catalysts)

| | |
|---|---|
| 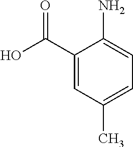 | (1) |

2-amino-5-methylbenzoic acid
(CAS No. 2941-78-8; Sigma-Aldrich)

| | |
|---|---|
| 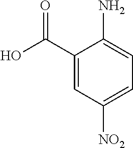 [SMILES: Nc1ccc([N+](=O)[O-])cc1C(=O)O] | (2) |

2-amino-5-nitrobenzoic acid
(CAS No. 616-79-5; Sigma-Aldrich)

| | |
|---|---|
| 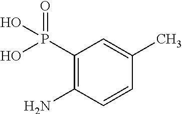 | (3) |

(2-amino-5-methylphenyl)phosphonic acid
(CAS 69675-98-5; Ambeed Inc.)

| | |
|---|---|
| 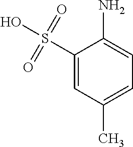 | (4) |

2-amino-5-methylbenzenesulfonic acid
(CAS No. 88-44-8; Sigma-Aldrich)

| | |
|---|---|
| 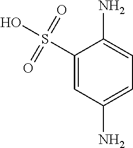 [SMILES: Nc1ccc(N)c(S(=O)(=O)O)c1] | (5) |

2,5-diaminobenzenesulfonic acid
(CAS No. 88-45-9; Sigma-Aldrich)

| | |
|---|---|
| 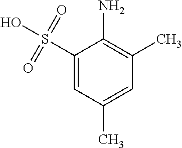 | (6) |

2-amino-3,5-dimethylbenzenesulfonic acid
(CAS No. 88-22-2; TCI Co. Ltd., Tokyo, JP)

| | |
|---|---|
| 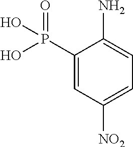 [SMILES: Nc1ccc([N+](=O)[O-])cc1P(=O)(O)O] | (7) |

(2-amino-5-nitrophenyl)phosphonic acid

| | |
|---|---|
| 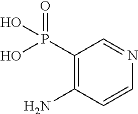 [SMILES: Nc1ccncc1P(=O)(O)O] | (8) |

(4-aminopyridin-3-yl)phosphonic acid

| | |
|---|---|
| 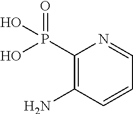 [SMILES: Nc1cccnc1P(=O)(O)O] | (9) |

(3-aminopyridin-2-yl)phosphonic acid

| | |
|---|---|
| 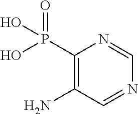 [SMILES: Nc1cncnc1P(=O)(O)O] | (10) |

(5-aminopyrimidin-4-yl)phosphonic acid

| | |
|---|---|
| 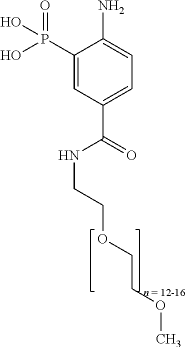 | (11) |

(2-amino-5-{[2-(2-poly-ethoxy)ethyl]carbamoyl}phenyl)phosphonic acid

| | |
|---|---|
| 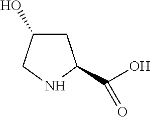 | (12) |

(2S,4R)-4-hydroxypyrrolidine-2-carboxylic acid
(“trans-4-hydroxy-L-proline;” CAS No. 51-35-4; Sigma-Aldrich)

| | |
|---|---|
| 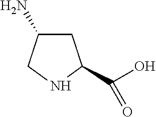 | (13) |

(2S,4R)-4-aminopyrrolidine-2-carboxylic acid
(“trans-4-aminoproline;” CAS No. 16257-88-8)

| | |
|---|---|
| 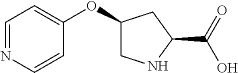 [SMILES: O=C(O)[C@@H]1C[C@H](Oc2ccncc2)CN1] | (14) |

(2S,4S)-4-[(pyridin-4-yl)oxy]pyrrolidine-2-carboxylic acid
(CAS No. 2309431-82-9; Enamine Ltd.)

| | |
|---|---|
| 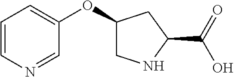 [SMILES: O=C(O)[C@@H]1C[C@H](Oc2cccnc2)CN1] | (15) |

(25,4S)-4-[(pyridin-3-yl)oxy]pyrrolidine-2-carboxylic acid
(“cis-m-O-Py-Pro”)

Compounds (1)-(6), (12), and (14) are commercially available. The compounds (7), (8), (9), (10), (11), (13), and (15) can be prepared from commercially available reagents using standard chemical synthesis techniques well-known in the art. See e.g., Crisalli et al., “Importance of ortho Proton Donors in Catalysis of Hydrazone Formation,” Org. Lett. 2013, 15, 7, 1646-1649.

Compounds (8) and (11) can be prepare by 2-step and 4-step syntheses, respectively, as described in Example 1. Briefly, in preparing compound (8), the compound, diethyl (4-aminopyridin-3-yl)phosphonate is prepared according to the procedure described in Guilard, R. et al. Synthesis, 2008, 10, 1575-1579. Then, the target compound (8), (4-aminopyridin-3-yl)phosphonic acid) is prepared by acid hydrolysis of the precursor compound of the diethyl (4-aminopyridin-3-yl)phosphonate. Compounds (9) and (10) can be prepared from similarly straightforward procedures.

In some examples, proteases may be used alone in un-fixing procedures. In some examples, catalysts may be used alone in un-fixing procedures. In some examples, one or more proteases may be used in combination with one or more catalysts in un-fixing procedures. In some examples, combinations of un-fixing agents may be used simultaneously in un-fixing procedures. In some examples, different un-fixing agents may be used consecutively in un-fixing procedures. In one example, a protease (e.g., Proteinase K) may be used in combination with Compound 1 and/or Compound 8). In another example, a cold active protease (e.g., subtilisin, such as subtilisin A) may be used in combination with Compound 1 and/or Compound 8).

Other reagents and methods can be used to reverse paraformaldehyde fixation including, without limitation, the un-fixing agents and methods described in PCT/US2020/066701, which is incorporated by reference herein in its entirety. In addition, reagents and methods for reversing other types of non-paraformaldehyde fixation can be used such as those described in PCT/US2020/066705, which is incorporated by reference herein in its entirety.

In some examples, un-fixing reactions may be performed on fixed cells as follows. Unattached cells are fixed with 4% PFA for 24 h at 4° C. and quenched with 10% Fetal Bovine Serum (“FBS”) in PBS. Un-fixing agents are prepared in a buffer at neutral pH. The concentration of the un-fixing agent(s) may be titrated to obtain desired results. The fixed cells are treated with the un-fixing agent solution, for example, at 40° C. for 2 hours. The reaction may also contain one or more RNase inhibitors. After the treatment, the reaction volume may be centrifuged to pellet the cells. Biomolecules (e.g., RNA) may be collected from the cell pellet and/or the supernatant of the centrifugation, using standard methods, and quantified. Success of the un-fixing, at least as it relates to RNA un-fixing, may be measured by both the amount of RNA that is recovered, and the ability of the recovered RNA to function as a substrate or template in various enzymatic reactions. In some examples, the ability of the recovered RNA to serve as template for production of suitable sequence libraries may be determined and serve as an indicator of the results obtained from the un-fixing reaction.

### Processing of Fixed Membrane Bound Particles

Fixed biological particles may be allocated into partitions. A partition may comprise the fixed biological particle or the fixed membrane bound particle. A plurality of fixed biological particles or fixed membrane bound particles may be partitioned into a plurality of partitions. The partitions may be partitions as described elsewhere herein, for example a droplet or well. The partitioning may be performed as described elsewhere herein. The partition may comprise additional reagents such as a cleaving agent or a barcode nucleic acid molecule. The barcode nucleic acid molecule may be a barcoded molecule as described elsewhere herein and may attached to a support, such as a bead, or other solid support described elsewhere herein. The barcode nucleic acid molecule may comprise a capture sequence or other sequence that is able to anneal to a nucleic acid from the fixed membrane bound particle. For example, the barcode nucleic acid molecule may comprise a poly-T sequence and may be able to anneal to an mRNA. The barcode nucleic acid molecule may comprise a unique molecular identifier (UMI) sequence or a sequencing primer sequence. For example, the barcode sequence may comprise a Read 1 sequence.

The partition may be subjected to a particular environment. The environment may allow for a reaction to proceed or decrease a probability for a reaction to occur. For example, a partition may be heated. The partition may be heated to allow for the cleavage agent to cleave bond or may increase the rate, efficiency, or efficacy of a cleaving agent. The partition may also be cooled or allowed to cool such to allow a reaction to occur. For example, a partition may be at a temperature that allows a first nucleic acid molecule to anneal to another nucleic acid molecule. For example, the partition may be at 25° C., 53° C., 70° C., or 90° C. For example, a partition may be subjected to a temperature of at least 5° C., 10° C., 15° C., 20° C., 25° C., 30° C., 35° C., 40° C., 45° C., 50° C., 55° C., 60° C., 65° C., 70° C., 75° C., 80° C., 85° C., 90° C., 95° C., 100° C., or higher. A partition may be subjected to a temperature of no higher than 5° C., 10° C., 15° C., 20° C., 25° C., 30° C., 35° C., 40° C., 45° C., 50° C., 55° C., 60° C., 65° C., 70° C., 75° C., 80° C., 85° C., 90° C., 95° C., 100° C., or lower. A partition may be subjected to a particular environment for a certain amount of time. For example, a partition may be subjected to a particular environment for 10 minutes, 15, minutes or 45 minutes. A partition may be subjected to a particular environment for at least 1, 2, 3, 4, 5, 6, 7, 8, 9, 10, 15, 20, 25, 30, 35, 40, 45, 50, 55, 60 minutes or more. A partition may be subjected to a particular environment for no more than 1, 2, 3, 4, 5, 6, 7, 8, 9, 10, 15, 20, 25, 30, 35, 40, 45, 50, 55, 60 minutes or less. A partition may be subjected to a particular environment indefinitely or new condition is initiated.

In various aspects, nucleic acid molecules from a partition may be released from the partition and may be subjected to additional reactions. It may be advantageous to perform a reaction either outside of a partition or inside of a partition. For example, a partition may allow molecules to interact without the interference of other molecules. A partition may cause a particular reagent to have a higher effective concentration which may be advantageous or disadvantageous for a particular reaction. In some cases, a reagent used for a reaction may prevent or decrease the efficacy of a second reaction. For example, a protease may be used for a cleaving reaction in a partition but may inhibit or impede a barcoding reaction (e.g., an enzymatic barcoding reaction) in the same partition. As such, releasing a molecule from a partition, via breaking the partition, or other method described elsewhere herein, for additional reaction(s) (e.g., an enzymatic barcoding reaction) may be performed. A reaction performed after releasing a nucleic acid molecule from a partition may comprise an extension reaction, amplification reaction, or ligation reaction. The reaction may be performed in the presence of a protease inhibitor.

In some aspects, the reactions may be performed or facilitated by an enzyme. For example, the enzyme may be a reverse transcriptase. The reverse transcriptase may be used to extend a nucleic acid molecule (e.g., a 3′ end of a nucleic acid molecule) to generate a nucleic acid barcode molecule. The reverse transcriptase may comprise RNase activity. The reverse transcriptase may append an additional sequence to the nucleic acid molecule. For example, the reverse transcriptase may append a poly-C sequence. The additional sequence may allow the annealing of another sequence such as a template switching oligo. The enzyme may be a ligase or a polymerase. The ligase may be used to ligate additional sequences to another nucleic acid. The ligase may be used to perform a blunt ligation or a splint ligation. A splint may be used in conjunction with a ligase such to ligate two molecules.

In various aspects, a barcoded nucleic acid molecule may be generated from the nucleic acid molecules originating from the fixed biological particle or the fixed membrane bound particle. The nucleic acid derived from or originating from the fixed biological particle or the fixed membrane bound particle may interact with a nucleic acid barcode molecule and a barcoded nucleic acid molecule may be generated by subjecting the molecules to a reaction, such as an extension reaction. In some cases, a template switching oligo may be used and the sequence of the template switching oligo may be appended or otherwise incorporated into the barcoded nucleic acid molecule. For example, a template-switching reverse transcriptase may be used in conjunction with a template switching oligo. The template switching oligo may allow the enzyme to continue to extend the nucleic acid molecule such that the full length of the nucleic acid molecule is generated, along with at least a portion of the sequence of the template switching oligo. Additionally, the template switching oligo or other sequence may be added to the barcoded nucleic acid via a ligation reaction, for example an RNA ligase-based (e.g., App Ligase based) ligation, or a splint ligation. Additionally, a polymerase and primer may be used to amplify the barcoded nucleic acid molecules. Additional sequences for attachment to flow cell of a sequencer, or other functional sequences may be added to a barcoded nucleic acid molecule. The barcoded nucleic acid may be subjected to additional reactions such as a sequencing reaction, or other reactions for generating a construct amenable to a sequenced on a particular sequencing platform.

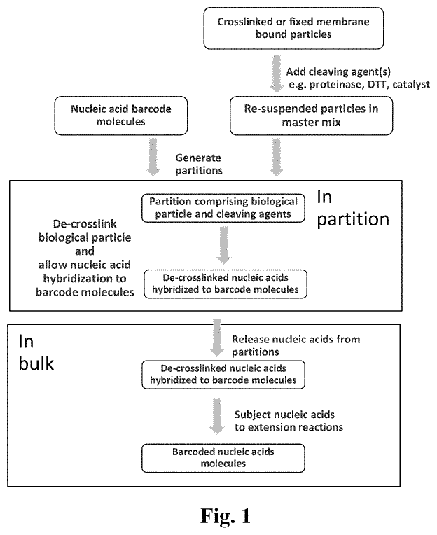

FIG. 1 shows an example workflow for processing crosslinked biological particles or crosslinked membrane bound particles. The biological particles or membrane bound particles may be crosslinked as described elsewhere herein and may be derived from a tissue or other sample. One or more cleaving agents such as proteinases, one or more reducing agents, e.g., dithiothreitol (DTT), and/or one or more catalysts may be added and the biological particles or membrane bound particles and one or more cleaving agents may be re-suspended in a master mix. Nucleic acid barcode molecules (such as those described elsewhere herein) may be added and partitions may be generated such the nucleic acid barcode molecules, crosslinked (or fixed) biological particles or membrane bound particles, and cleaving agents are co-partitioned. The partition may be subjected to a temperature or other environmental condition such that the cleaving agents may be activated. The one or more cleaving agents may cleave bonds (e.g. de-crosslink) and allow the nucleic acid molecules derived from the crosslinked (or fixed) biological particles or membrane bound particles to interact with the nucleic acid barcode molecules. The environment may be altered such that a nucleic acid molecule derived from the crosslinked (or fixed) biological particles or membrane bound particles and a nucleic acid barcode molecule are annealed to one other and are non-covalently bound. The nucleic acid molecule may then be released from the partition, for example via the breaking of the partition, and a nucleic acid derived from the crosslinked (or fixed) biological particles or membrane bound particles and a nucleic acid barcode molecule remain annealed to one another. The nucleic acid molecules may be subjected to an extension reaction such a new barcoded nucleic acid is generated from the nucleic acid molecule derived from the crosslinked (or fixed) biological particles or membrane bound particles and a nucleic acid barcode molecule. The extension reactions may be performed in the presence of the protease inhibitor, which may inhibit the activity of proteases that were used to de-crosslink. The release of the nucleic acid molecules from the partition may also lower the effective concentration of proteases and allow enzymes to perform the extension reactions, without a potentially detrimental activity of the proteases. In some examples, the nucleic acid molecule may not be released from the partition. In some examples, extension reactions may be performed within the partition.

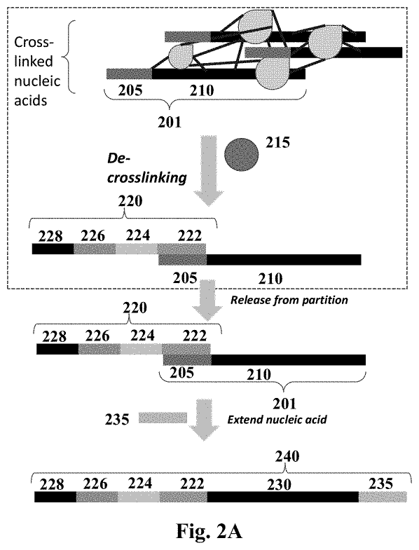

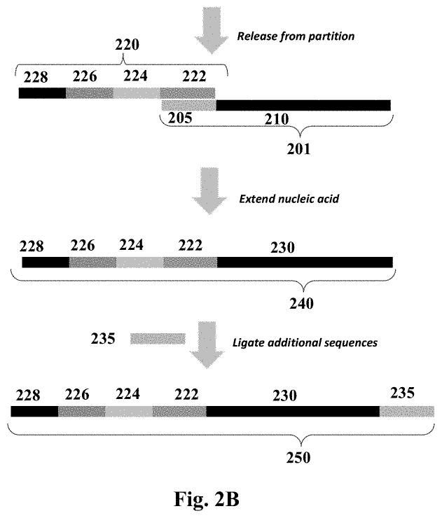

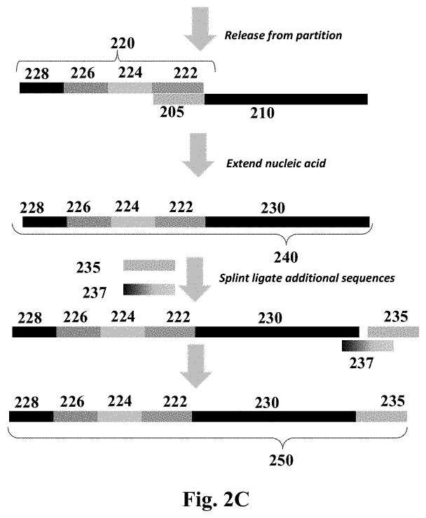

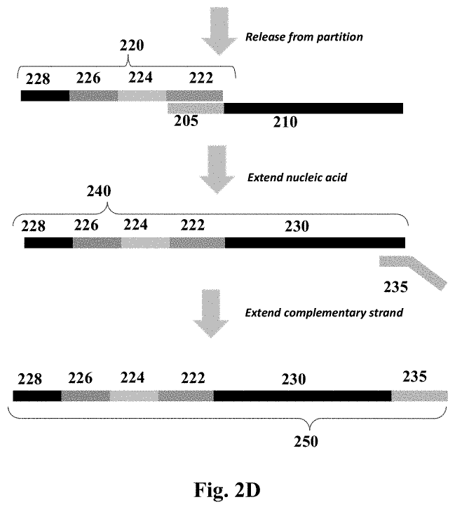

FIGS. 2A-D show additional example schematics for generating barcoded nucleic acid molecules. Crosslinked nucleic acid molecules may be present in a fixed membrane bound particles. FIG. 2A shows the generation of a barcoded nucleic acid molecule from a crosslinked nucleic acid. A crosslinked nucleic acid molecule 201 may comprise a nucleic acid sequence 210 and a capturable sequence 205. For example, the nucleic acid molecule 201 may be an mRNA and the capturable sequence 205 may be a poly-A sequence. The crosslinked nucleic acid molecules may be less accessible to interact with a nucleic acid barcode molecule such as nucleic acid barcode molecule 220. The cross-linked nucleic acid molecule may be de-crosslinked by cleaving agents 215. The cleaving agents comprise proteases, catalysts, or a change in an environment, for example, heating the partition. The de-crosslinked nucleic acid molecules may be able to interact with nucleic acid barcode molecule 220. The nucleic acid barcode molecule 220 may comprise multiple function sequences 222, 224, 226, and 222. 228 may be a sequencing primer sequence, 226 may be a barcode sequence and 224 may be a UMI sequence. Sequence 222 may be a capture sequence or a sequence that is able to anneal to a nucleic acid membrane derived from the fixed membrane bound particle. Sequence 222 may be complementary to sequence 205 of a de-crosslinked nucleic acid molecule. Upon annealing nucleic acid barcode molecule 220 to 201, the temperature of the partition may be lowered such that nucleic acid barcode molecule 220 and nucleic acid molecule 201 remain annealed. The nucleic acid molecule may then be released from the partition, for example via the breaking of the partition, and the nucleic acid molecules may remain annealed in the bulk solution. The nucleic acid complex may be subjected to an extension reaction. The nucleic acid barcode molecule 220 may be extended using reverse transcriptase with template switching activity. A template switching oligo 235 may be used along with the reverse transcriptase. The reverse transcriptase may extend the nucleic acid barcode molecule 220 such to generate a complementary sequence of sequence 210 of the nucleic acid molecule derived from the fixed membrane particle to generate a sequence 230. Additionally, the reverse transcriptase may template switch on to the template switching oligo and generate a sequence 235 corresponding to the template switching oligo sequence, or complement thereof, to generate nucleic acid molecule 240. Nucleic acid molecule 240 may be subjected to downstream processes such as sequencing to identify the sequence of the nucleic acid molecule. In some examples, the nucleic acid molecule may not be released from the partition. In some examples, extension reactions may be performed within the partition. In some examples, reverse transcriptase reactions may be performed within the partition.

FIG. 2B shows another example scheme of barcoding a de-crosslinked nucleic acid molecule. As shown in FIG. 2B, a de-crosslinked nucleic acid molecule 201 may interact with a nucleic acid barcode molecule 220. The de-crosslinked nucleic acid molecule 201 and nucleic acid barcode molecule 220 may then be released from the partition. The nucleic acid complex may be subjected to an extension reaction. The nucleic acid barcode molecule 220 may be extended using reverse transcriptase. The reverse may comprise RNase activity such that the template may be digested. The reverse transcriptase may extend the nucleic acid barcode molecule to add a sequence 230 complementary to nucleic acid molecule 201 and generate nucleic acid molecule 240. Additional sequences may be ligated to nucleic acid molecule 240. The additional sequences may be functional sequences such to sequencing primer sequences or sequences for attaching the nucleic acid molecules to a flow cell. A ligation reaction may be performed on nucleic acid molecule 240. Nucleic acid molecule 235 comprising a functional sequence may be ligated to nucleic acid molecule 240 to generate nucleic acid molecule 250. The reaction may be facilitated by a ligase. The ligase and nucleic acid molecule 235 may be specific such that the ligation reaction has some specificity. For example, the ligase may be an App ligase, and nucleic acid 235 may comprise a 5′ adenylated sequence. Nucleic acid molecule 250 may be subjected to downstream processes such as sequencing to identify the sequence of the nucleic acid.

FIG. 2C shows another example scheme of barcoding a de-crosslinked nucleic acid molecule. As shown in FIG. 2C, a de-crosslinked nucleic acid molecule 201 may interact with a nucleic acid barcode molecule 220. The de-crosslinked nucleic acid molecule 201 and nucleic acid barcode molecule 220 may then be released from the partition. The nucleic acid complex may be subjected to an extension reaction. The nucleic acid barcode molecule 220 may be extended using reverse transcriptase. The reverse may comprise RNase activity such that the template may be digested. The reverse transcriptase may extend the nucleic acid barcode molecule to add a sequence 230 complementary to nucleic acid molecule 201 and generate nucleic acid molecule 240. Additional sequences may be ligated to nucleic acid molecule 240. The additional sequences may be functional sequences such to sequencing primer sequences or sequences for attaching the nucleic acid molecules to a flow cell. A ligation reaction may be performed on nucleic acid molecule 240. Nucleic acid molecule 235 comprising a functional sequence may be ligated to nucleic acid molecule 240 to generate nucleic acid 250. The reaction may be facilitated by a splint nucleic acid molecule 237 and a ligase. The splint may comprise sequences that are complementary to sequence 230 and a sequence complementary to nucleic acid 235. The splint nucleic acid molecule may allow the ligation to be specific and ligate nucleic acid molecule 240 to nucleic acid molecule 235 and generate nucleic acid molecule 250. Nucleic acid molecule 250 may be subjected to downstream processes such as sequencing to identify the sequence of the nucleic acid.

FIG. 2D shows another example scheme of barcoding a de-crosslinked nucleic acid molecule. As shown in FIG. 2D, a de-crosslinked nucleic acid molecule 201 may interact with a nucleic acid barcode molecule 220. The de-crosslinked nucleic acid molecule 201 and nucleic acid barcode molecule 220 may then be released from the partition. The nucleic acid complex may be subjected to an extension reaction. The nucleic acid barcode molecule 220 may be extended using reverse transcriptase. The reverse may comprise RNase activity such that the template may be digested. The reverse transcriptase may extend the nucleic acid barcode molecule to add a sequence 230 complementary to nucleic acid molecule 201 and generate nucleic acid molecule 240. Additional sequences may be appended to nucleic acid molecule 240. The additional sequences may be functional sequences such to sequencing primer sequences or sequences for attaching the nucleic acid molecules to a flow cell. An extension reaction may be performed on nucleic acid molecule 240. Nucleic acid molecule 235 comprising a functional sequence may be used as a primer and anneal to nucleic acid molecule 240 to generate nucleic acid molecule 250. The reaction may be facilitated by a polymerase. The primer may comprise sequences that are complementary to sequence 230. The primer may comprise a random n-mer or comprise a random sequence that may anneal to nucleic acid molecule 240. Nucleic acid molecule 250 may be subjected to downstream processes such as sequencing to identify the sequence of the nucleic acid.

### Systems and Methods for Sample Compartmentalization (Partitioning)

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. The partition can be a droplet in an emulsion. A partition may comprise one or more other partitions.

A partition may include one or more particles. A partition may include one or more types of particles. For example, a partition of the present disclosure may comprise one or more biological particles and/or macromolecular constituents thereof. A partition may comprise one or more gel beads. A partition may comprise one or more cell beads. A partition may include a single gel bead, a single cell bead, or both a single cell bead and single gel bead. A partition may include one or more reagents. Alternatively, a partition may be unoccupied. For example, a partition may not comprise a bead. A cell bead can be a biological particle and/or one or more of its macromolecular constituents encased inside of a gel or polymer matrix, such as via polymerization of a droplet containing the biological particle and precursors capable of being polymerized or gelled. Unique identifiers, such as barcodes, may be injected into the droplets previous to, subsequent to, or concurrently with droplet generation, such as via a support (e.g., bead), as described elsewhere herein. Microfluidic channel networks (e.g., on a chip) can be utilized to generate partitions as described herein. Alternative mechanisms may also be employed in the partitioning of individual biological particles, including porous membranes through which aqueous mixtures of cells are extruded into non-aqueous fluids.

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

In some instances, a droplet is formed by creating an emulsion by mixing or agitating immiscible phases. Mixing or agitation may comprise various agitation techniques, such as vortexing, pipetting, tube flicking, or other agitation techniques. In some cases, mixing or agitation may be performed without using a microfluidic device. In some examples, a droplet may be formed by exposing a mixture to ultrasound or sonication. For example, to partition contents into droplets, a mixture comprising a first fluid, a second fluid, optionally a surfactant, and the contents can be subject to such agitation techniques to generate a plurality of droplets (first fluid-in-second fluid or second fluid-in-first fluid) comprising the contents, or subsets thereof. In an example, a mixture comprises beads. Upon agitation, the beads in the mixture may limit droplet break-up into droplets smaller than the size of the beads, and a substantially monodisperse population of droplets comprising the beads may result.

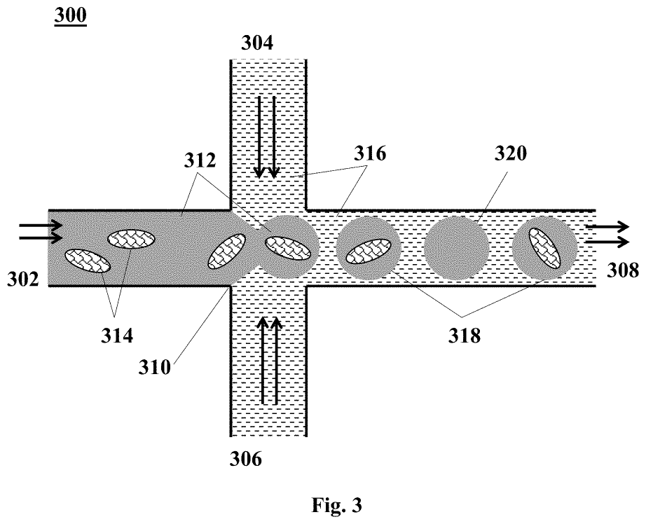

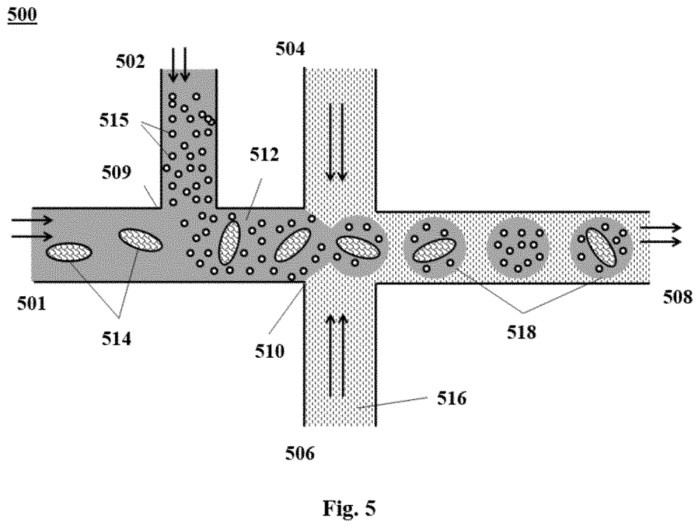

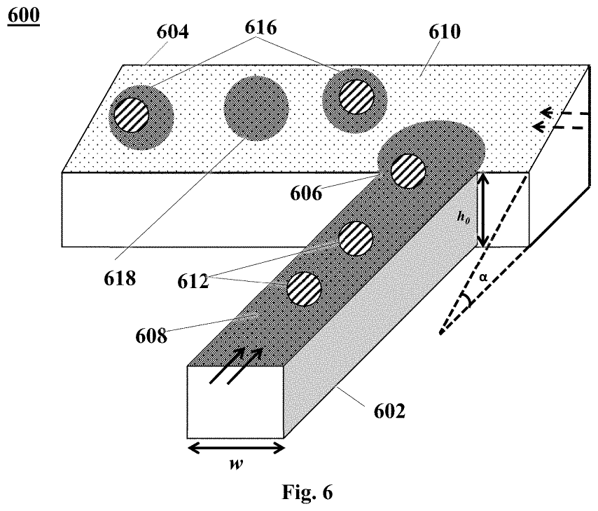

In the case of droplets in an emulsion, allocating individual particles to discrete partitions may in one non-limiting example be accomplished by introducing a flowing stream of particles in an aqueous fluid into a flowing stream or reservoir of a non-aqueous fluid, such that droplets are generated at the junction of the two streams (see generally, e.g., FIGS. 3 and 6). Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition in order to ensure that those partitions that are occupied are primarily singly occupied. In some cases, partitions among a plurality of partitions may contain at most one biological particle (e.g., bead, DNA, cell, nucleus, or cellular material). In some embodiments, the various parameters (e.g., fluid properties, particle properties, microfluidic architectures, etc.) may be selected or adjusted such that most partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

FIG. 3 shows an example of a microfluidic channel structure 300 for partitioning individual biological particles. The channel structure 300 can include channel segments 302, 304, 306 and 308 communicating at a channel junction 310. In operation, a first aqueous fluid 312 that includes suspended biological particles (or cells or nuclei) 314 may be transported along channel segment 302 into junction 310, while a second fluid 316 that is immiscible with the aqueous fluid 312 is delivered to the junction 310 from each of channel segments 304 and 306 to create discrete droplets 318, 320 of the first aqueous fluid 312 flowing into channel segment 308, and flowing away from junction 310. The channel segment 308 may be fluidically coupled to an outlet reservoir where the discrete droplets can be stored and/or harvested. A discrete droplet generated may include an individual biological particle 314 (such as droplets 318). A discrete droplet generated may include more than one individual biological particle 314 (not shown in FIG. 3). A discrete droplet may contain no biological particle 314 (such as droplet 320). Each discrete partition may maintain separation of its own contents (e.g., individual biological particle 314) from the contents of other partitions.

The second fluid 316 can comprise an oil, such as a fluorinated oil, that includes a fluorosurfactant for stabilizing the resulting droplets, for example, inhibiting subsequent coalescence of the resulting droplets 318, 320. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 300 may have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 318, containing one or more biological particles 314, and (2) unoccupied droplets 320, not containing any biological particles 314. Occupied droplets 318 may comprise singly occupied droplets (having one biological particle) and multiply occupied droplets (having more than one biological particle). As described elsewhere herein, in some cases, most occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it may be desirable to minimize the creation of excessive numbers of empty partitions, such as to reduce costs and/or increase efficiency. While this minimization may be achieved by providing a sufficient number of biological particles (e.g., biological particles 314) at the partitioning junction 310, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles (e.g., in channel segment 302), or other fluids directed into the partitioning junction (e.g., in channel segments 304, 306) can be controlled such that, in many cases, no more than about 50% of the generated partitions, no more than about 25% of the generated partitions, or no more than about 10% of the generated partitions are unoccupied. These flows can be controlled to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

As will be appreciated, the above-described occupancy rates are also applicable to partitions that include both biological particles and additional reagents, including, but not limited to, supports or beads (e.g., gel beads) carrying barcoded nucleic acid molecules (e.g., oligonucleotides) (described in relation to FIGS. 3 and 6). The occupied partitions (e.g., at least about 10%, 20%, 30%, 40%, 50%, 60%, 70%, 80%, 90%, 95%, or 99% of the occupied partitions) can include both a support (e.g., bead) comprising barcoded nucleic acid molecules and a biological particle.

As above, in at least one embodiment, the methods comprise generating a discrete droplet encapsulating the biological sample. In at least one embodiment, the method further comprises generating a discrete droplet encapsulating the fixed biological sample and one or more un-fixing agents.

In at least one embodiment wherein the method comprises generating a discrete droplet, the discrete droplet further comprises assay reagents; optionally, wherein the assay reagents are contained in a bead. In at least one embodiment, the discrete droplet further comprises a barcode; optionally, wherein the barcode is contained in a bead.

In some cases, the droplets among a plurality of discrete droplets formed contain at most one particle (e.g., one bead, one cell, one nucleus). The flows and microfluidic channel architectures also can be controlled to ensure a given number of singly occupied droplets, less than a certain level of unoccupied droplets, and/or less than a certain level of multiply occupied droplets.

In another aspect of the disclosure, fixed cells or nuclei, protease composition, and optional un-fixing agent composition may then be partitioned (e.g., in a droplet or well) with other reagents for processing of one or more analytes as described herein. In one embodiment, the fixed cell or nucleus, protease composition, and optional un-fixing agent composition may be partitioned with a support (e.g., a bead) comprising nucleic acid molecules suitable for barcoding of the one or more analytes. In another embodiment, the nucleic acid molecules may include nucleic acid sequences that provide identifying information, e.g., barcode sequence(s).

The inclusion of a barcode in a discrete droplet along with the biological sample provides a unique identifier that allows data from the biological sample to be distinguished and individually analyzed. Barcodes can be delivered before, after, or concurrent with the biological sample in discrete droplet. For example, barcodes may be injected into droplets before, after, or concurrently with droplet generation. Barcodes useful in the methods of the present disclosure typically comprise a nucleic acid molecule (e.g., an oligonucleotide). The nucleic acid barcode molecules typically are delivered to a partition via a support, such as bead. In some cases, barcode nucleic acid molecules are initially associated with the bead upon generation of the discrete droplet, and then released from the bead upon application of a stimulus to droplet. Barcode carrying beads useful in the methods of the present disclosure are described in further detail elsewhere herein.

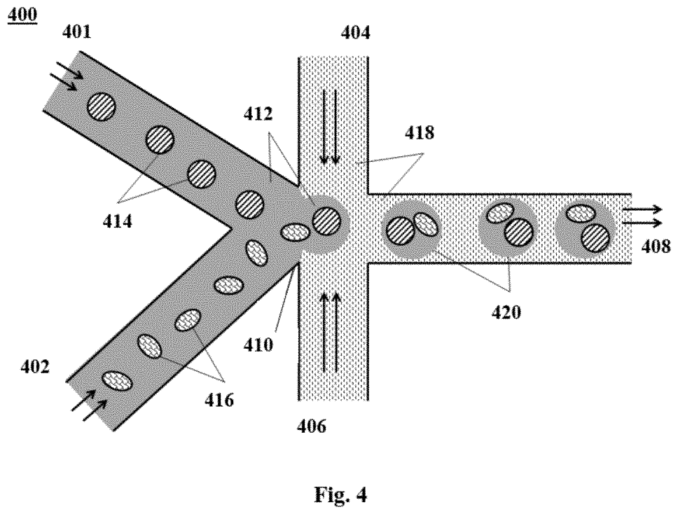

FIG. 4 shows an exemplary microfluidic channel structure 400 for generating discrete droplets encapsulating a barcode carrying bead 414 along with a biological sample particle 416. The channel structure 400 includes channel segments 401, 402, 404, 406 and 408 in fluid communication at a channel junction 410. In operation, the channel segment 401 transports an aqueous fluid 412 that can include a plurality of beads 414 (e.g., gel beads carrying barcode oligonucleotides) along the channel segment 401 into junction 410. The plurality of beads 414 may be sourced from a suspension of beads. For example, the channel segment 401 can be connected to a reservoir comprising an aqueous suspension of beads 414. The channel segment 402 transports the aqueous fluid 412 that includes a plurality of biological sample particles 416 along the channel segment 402 into junction 410. The plurality of biological sample particles 416 may be sourced from a suspension of biological sample particles. For example, the channel segment 402 may be connected to a reservoir comprising an aqueous suspension of biological sample particles 416. In some instances, the aqueous fluid 412 in either the first channel segment 401 or the second channel segment 402, or in both segments, can include one or more reagents, as further described elsewhere herein. For example, in some embodiments of the present disclosure, where the biological sample particles are fixed biological sample particles, the aqueous fluid in the first and/or second channel segments that delivers the biological sample and beads, respectively, can include an un-fixing agent. The second fluid 418 that is immiscible with the aqueous fluid 412 is delivered to the junction 410 from each of channel segments 404 and 406. Upon meeting of the aqueous fluid 412 from each of channel segments 401 and 402 and the second fluid 418 (e.g., a fluorinated oil) from each of channel segments 404 and 406 at the channel junction 410, the aqueous fluid 412 is partitioned into discrete droplets 420 in the second fluid 418 and flow away from the junction 410 along channel segment 408. The channel segment 408 can then deliver the discrete droplets encapsulating the biological sample particle and barcode carrying bead to an outlet reservoir fluidly coupled to the channel segment 408, where they can be collected.

As an alternative, the channel segments 401 and 402 may meet at another junction upstream of the junction 410. At such junction, beads and biological particles may form a mixture that is directed along another channel to the junction 410 to yield droplets 420. The mixture may provide the beads and biological particles in an alternating fashion, such that, for example, a droplet comprises a single bead and a single biological particle.

Using such a channel system as exemplified in FIG. 4, discrete droplets 420 can be generated that encapsulate an individual particle of a biological sample, and one bead, wherein the bead can carry a barcode and/or another reagent. It is also contemplated, that in some instances, a discrete droplet may be generated using the channel system of FIG. 4, wherein droplet includes more than one individual biological sample particle or includes no biological sample. Similarly, in some embodiments, the discrete droplet may include more than one bead or no bead. A discrete droplet also may be completely unoccupied (e.g., no bead or biological sample).

In some embodiments, it is desired that the beads, biological sample particles, and generated discrete droplets flow along channels at substantially regular flow rates that generate a discrete droplet containing a single bead and a single biological sample particle. Regular flow rates and devices that may be used to provide such regular flow rates are known in the art, see e.g., U.S. Patent Publication No. 2015/0292988, which is hereby incorporated by reference herein in its entirety. In some embodiments, the flow rates are set to provide discrete droplets containing a single bead and a biological sample particle with a yield rate of greater than 5%, 10%, 20%, 30%, 40%, 50%, 60%, 70%, 80%, 90%, or 95%.

Beads that can carry barcodes and/or other reagents that are useful with the methods of the present disclosure can include beads that are porous, non-porous, solid, semi-solid, semi-fluidic, fluidic, and/or a combination thereof. In some embodiments, the bead can be made of a material that is dissolvable, disruptable, and/or degradable, such as a gel bead comprising a hydrogel. Alternatively, in some embodiments, the bead is not degradable.

Although FIG. 3 and FIG. 4 have been described in terms of providing substantially singly occupied discrete droplets, it is also contemplated in certain embodiments that it is desirable to provide multiply occupied discrete droplets, e.g., a single droplet that contains two, three, four or more cells or nuclei from a biological sample, and/or multiple different supports, such as a bead carrying a barcode nucleic acid molecule and/or a bead carrying a reagent such as an un-fixing agent or assay reagent. Accordingly, as noted elsewhere herein, the flow characteristics of the biological particle and/or the beads can be controlled to provide for such multiply occupied droplets. In particular, the flow parameters of the liquids used in the channel structures may be controlled to provide a given droplet occupancy rate greater than about 50%, greater than about 75%, and in some cases greater than about 80%, 90%, 95%, or higher.

In some embodiments, the beads useful in the methods of the present disclosure are supports (e.g., beads) capable of delivering reagents (e.g., an un-fixing agent, and/or an assay reagent) into the discrete droplet generated containing the biological sample particle. In some embodiments, the different beads (e.g., containing different reagents) can be introduced from different sources into different inlets leading to a common droplet generation junction (e.g., junction 410). In such cases, the flow and frequency of the different beads into the channel or junction may be controlled to provide for a certain ratio supports (e.g., beads) from each source, while ensuring a given pairing or combination of such beads into a partition with a given number of biological particles (e.g., one biological particle and one bead per partition).

The discrete droplets described herein generally comprise small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less. In some embodiments, the discrete droplets generated that encapsulate a biological sample particle have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. It will be appreciated that the sample fluid volume, e.g., including co-partitioned biological particles and/or beads, within the droplets may be less than about 90% of the above described volumes, less than about 80%, less than about 70%, less than about 60%, less than about 50%, less than about 40%, less than about 30%, less than about 20%, or less than about 10% of the above described volumes.

The methods of generating discrete droplets useful with the methods of the present disclosure, result in the generation of a population or plurality of discrete droplets containing a biological sample particle (e.g., a fixed biological sample) and other reagents (e.g., an un-fixing agent). Generally, the methods are easily controlled to provide for any suitable number of droplets. For example, at least about 1,000 discrete droplets, at least about 5,000 discrete droplets, at least about 10,000 discrete droplets, at least about 50,000 discrete droplets, at least about 100,000 discrete droplets, at least about 500,000 discrete droplets, at least about 1,000,000 discrete droplets, at least about 5,000,000 discrete droplets, at least about 10,000,000 discrete droplets, or more discrete droplets can be generated or otherwise provided. Moreover, the plurality of discrete droplets may comprise both unoccupied and occupied droplets.

As described elsewhere herein, in some embodiments of the methods of the present disclosure, the generated discrete droplets encapsulating a biological sample particle, and optionally, one or more different beads, also contain other reagents. In some embodiments, the other reagents encapsulated in the droplet include lysis and/or un-fixing agents that act to release and/or un-fix the biomolecule contents of the biological sample particle within the droplet. In some embodiments, the lysis and/or un-fixing agents can be contacted with the biological sample suspension concurrently with, or immediately prior to, the introduction of the biological sample particles into the droplet generation junction of the microfluidic system (e.g., junction 410). In some embodiments, the agents are introduced through an additional channel or channels upstream of the channel junction.

In some embodiments, a biological sample particle can be co-partitioned along with the other reagents. FIG. 5 shows an example of a microfluidic channel structure 500 for co-partitioning biological sample particles and other reagents, including lysis and/or un-fixing agents. The channel structure 500 can include channel segments 501, 502, 504, 506 and 508. Channel segments 501 and 502 communicate at a first channel junction 509. Channel segments 502, 504, 506, and 508 communicate at a second channel junction 510. In exemplary co-partitioning operation, the channel segment 501 may transport an aqueous fluid 512 that includes a plurality of biological sample particles 514 (e.g., a fixed biological sample) along the channel segment 501 into the second junction 510. As an alternative or in addition to, channel segment 501 may transport beads (e.g., beads that carry barcodes). For example, the channel segment 501 may be connected to a reservoir comprising an aqueous suspension of biological sample particles 514. Upstream of, and immediately prior to reaching, the second junction 510, the channel segment 501 may meet the channel segment 502 at the first junction 509. The channel segment 502 can transport a plurality of reagents 515 (e.g., lysis or un-fixing agents) in the aqueous fluid 512 along the channel segment 502 into the first junction 509. For example, the channel segment 502 may be connected to a reservoir comprising the reagents 515. After the first junction 509, the aqueous fluid 512 in the channel segment 501 can carry both the biological sample particles 514 and the reagents 515 towards the second junction 510. In some instances, the aqueous fluid 512 in the channel segment 501 can include one or more reagents, which can be the same or different reagents as the reagents 515. A second fluid 516 that is immiscible with the aqueous fluid 512 (e.g., a fluorinated oil) can be delivered to the second junction 510 from each of channel segments 504 and 506. Upon meeting of the aqueous fluid 512 from the channel segment 501 and the second fluid 516 from each of channel segments 504 and 506 at the second channel junction 510, the aqueous fluid 512 is partitioned as discrete droplets 518 in the second fluid 516 and flow away from the second junction 510 along channel segment 508. The channel segment 508 may deliver the discrete droplets 518 to an outlet reservoir fluidly coupled to the channel segment 508, where they may be collected for further analysis.

Discrete droplets generated can include an individual biological sample particle 514 and/or one or more reagents 515, depending on what reagents are included in channel segment 502. In some instances, a discrete droplet generated may also include a barcode carrying bead (not shown), such as can be added via other channel structures described elsewhere herein. In some instances, a discrete droplet may be unoccupied (e.g., no reagents, no biological particles). Generally, the channel segments described herein may be coupled to any of a variety of different fluid sources or receiving components, including reservoirs, tubing, manifolds, or fluidic components of other systems. As will be appreciated, the microfluidic channel structure 500 may have other geometries. For example, a microfluidic channel structure can have more than two channel junctions. For example, a microfluidic channel structure can have 2, 3, 4, 5 channel segments or more each carrying the same or different types of beads, reagents, and/or biological sample particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electro-kinetic pumping, vacuum, capillary or gravity flow, or the like.

FIG. 6 shows an example of a microfluidic channel structure for the controlled partitioning of beads into discrete droplets. A channel structure 600 can include a channel segment 602 communicating at a channel junction 606 (or intersection) with a reservoir 604. The reservoir 604 can be a chamber. Any reference to “reservoir,” as used herein, can also refer to a “chamber.” In operation, an aqueous fluid 608 that includes suspended beads 612 may be transported along the channel segment 602 into the junction 606 to meet a second fluid 610 that is immiscible with the aqueous fluid 608 in the reservoir 604 to create droplets 616, 618 of the aqueous fluid 608 flowing into the reservoir 604. At the junction 606 where the aqueous fluid 608 and the second fluid 610 meet, droplets can form based on factors such as the hydrodynamic forces at the junction 606, flow rates of the two fluids 608, 610, fluid properties, and certain geometric parameters (e.g., w, h0, α, etc.) of the channel structure 600. A plurality of droplets can be collected in the reservoir 604 by continuously injecting the aqueous fluid 608 from the channel segment 602 through the junction 606.

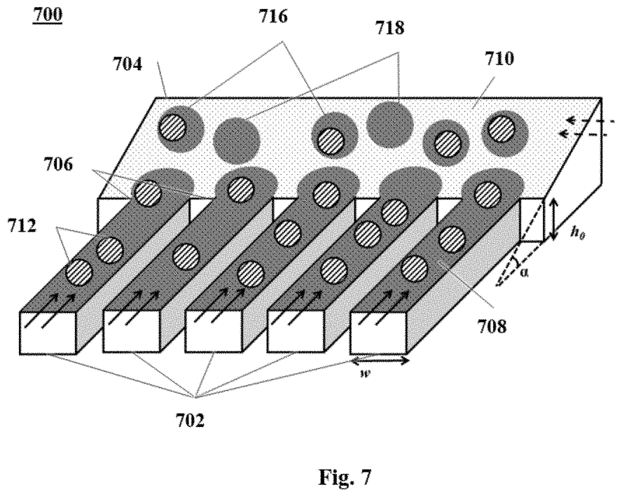

FIG. 7 shows an example of a microfluidic channel structure for increased droplet generation throughput. A microfluidic channel structure 700 can comprise a plurality of channel segments 702 and a reservoir 704. Each of the plurality of channel segments 702 may be in fluid communication with the reservoir 704. The channel structure 700 can comprise a plurality of channel junctions 706 between the plurality of channel segments 702 and the reservoir 704. Each channel junction can be a point of droplet generation. The channel segment 602 from the channel structure 600 in FIG. 6 and any description to the components thereof may correspond to a given channel segment of the plurality of channel segments 702 in channel structure 700 and any description to the corresponding components thereof. The reservoir 604 from the channel structure 600 and any description to the components thereof may correspond to the reservoir 704 from the channel structure 700 and any description to the corresponding components thereof.

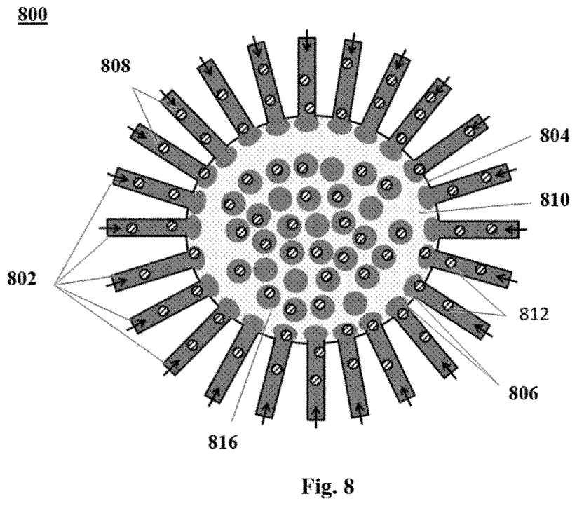

FIG. 8 shows another example of a microfluidic channel structure for increased droplet generation throughput. A microfluidic channel structure 800 can comprise a plurality of channel segments 802 arranged generally circularly around the perimeter of a reservoir 804. Each of the plurality of channel segments 802 may be in fluid communication with the reservoir 804. The channel structure 800 can comprise a plurality of channel junctions 806 between the plurality of channel segments 802 and the reservoir 804. Each channel junction can be a point of droplet generation. The channel segment 602 from the channel structure 600 in FIG. 6 and any description to the components thereof may correspond to a given channel segment of the plurality of channel segments 802 in channel structure 800 and any description to the corresponding components thereof. The reservoir 604 from the channel structure 600 and any description to the components thereof may correspond to the reservoir 804 from the channel structure 800 and any description to the corresponding components thereof. Additional aspects of the microfluidic structures depicted in FIGS. 6, 7 and 8, including systems and methods implementing the same, are provided in US Published Patent Application No 20190323088, which is incorporated herein by reference in its entirety.

Once the lysis and/or un-fixing agents are co-partitioned in a droplet with a fixed biological sample particle, these reagents can facilitate the release and un-fixing of the biomolecular contents of the biological sample particle within the droplet. As described elsewhere herein, the un-fixed biomolecular contents released in a droplet remain discrete from the contents of other droplets, thereby allowing for detection and quantitation of the biomolecular analytes of interest present in that distinct biological sample. As further described herein, lysis agents may also be used in partitions.

In addition to the lysis and/or un-fixing agents co-partitioned into discrete droplets with the biological sample particles, it is further contemplated that other assay reagents can also be co-partitioned in the droplet. For example, DNase and RNase inactivating agents or inhibitors, such as proteinase K, chelating agents, such as EDTA, proteases, such as subtilisin A, and other reagents employed in removing or otherwise reducing negative activity or impact of different cell lysate components on subsequent processing of nucleic acids.

In some embodiments, the biological sample particles encapsulated in discrete droplets with other reagents are exposed to an appropriate stimulus to release the biomolecular contents of the sample particles and/or the contents of a co-partitioned support (e.g., a bead). For example, in some embodiments, a chemical stimulus may be co-partitioned in the droplet along with a biological sample particle and a bead (e.g., gel bead) to allow for the degradation of the bead and release of its contents into the droplet. In some embodiments, a discrete droplet can be generated with a fixed biological sample particle and an un-fixing agent, wherein the un-fixing agent is contained in a support (e.g., a bead) that can be degraded by heat stimulus. In such an embodiment, the droplet is exposed to heat stimulus thereby degrading the bead and releasing the un-fixing agent. In another embodiment, it is contemplated that a droplet encapsulating a fixed biological sample particle, and two different beads (e.g., one bead carrying an un-fixing agent, and one bead carrying assay reagents), wherein the contents of the two different beads are released by non-overlapping stimuli (e.g., a chemical stimulus and a heat stimulus). Such an embodiment can allow the release of the different reagents into the same discrete droplet at different times. For example, a first bead, triggered by heat stimulus, releases an un-fixing agent into the droplet, and then after a set time, a second bead, triggered by a chemical stimulus, releases assay reagents that detect analytes of the un-fixed biological sample particle.

Additional assay reagents may also be co-partitioned into discrete droplets with the biological samples, such as endonucleases to fragment a biological sample's DNA, DNA polymerase enzymes and dNTPs used to amplify the biological sample's nucleic acid fragments and to attach the barcode molecular tags to the amplified fragments. Other enzymes may be co-partitioned, including without limitation, polymerase, transposase, ligase, proteinase K, DNase, subtilisin A, etc. Additional assay reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching.

In some embodiments, template switching can be used to increase the length of cDNA generated in an assay. In some embodiments, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner.

Once the contents of a biological sample cell or nucleus are released into a discrete droplet, the biomolecular components (e.g., macromolecular constituents of biological samples, such as RNA, DNA, or proteins) contained therein may be further processed within the droplet. In accordance with the methods and systems described herein, the biomolecular contents of individual biological samples can be provided with unique barcode identifiers, and upon characterization of the biomolecular components (e.g., in a sequencing assay) they may be attributed as having been derived from the same biological sample. The ability to attribute characteristics to individual biological samples or groups of biological samples is provided by the assignment of a nucleic acid barcode sequence specifically to an individual biological sample or groups of biological samples.

In some aspects, the unique identifier barcodes are provided in the form of nucleic acid molecules (e.g., oligonucleotides) that comprise sequences that may be attached to or otherwise associated with the nucleic acid contents of individual biological sample, or to other components of the biological sample, and particularly to fragments of those nucleic acids. In some embodiments, only one nucleic acid barcode sequence is associated with a given discrete droplet, although in some cases, two or more different barcode sequences may be present. The nucleic acid barcode sequences can include from about 6 to about 20 or more nucleotides within the sequence of the nucleic acid molecules (e.g., oligonucleotides). In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

In some embodiments, the nucleic acid barcode molecules can also comprise other functional sequences useful in the processing of the nucleic acids from the biological sample in the droplet. These functional sequences can include, e.g., targeted or random/universal amplification primer sequences for amplifying the nucleic acid molecules from the individual biological samples within the partitions while attaching the associated barcode sequences, sequencing primers or primer recognition sites, hybridization or probing sequences, e.g., for identification of presence of the sequences or for pulling down barcoded nucleic acid molecules, or any of a number of other potential functional sequences.

### Beads and Barcode Molecules

Nucleic acid barcode molecules may be delivered to a partition (e.g., a droplet or well) via a support (e.g., a solid support) or carrier (e.g., a bead). In some cases, nucleic acid barcode molecules are initially associated with the support and then released from the support upon application of a stimulus, which allows the nucleic acid barcode molecules to dissociate or to be released from the support. In specific examples, nucleic acid barcode molecules are initially associated with the support (e.g., bead) and then released from the support upon application of a biological stimulus, a chemical stimulus, a thermal stimulus, an electrical stimulus, a magnetic stimulus, and/or a photo stimulus.

A nucleic acid barcode molecule may contain a barcode sequence and a functional sequence, such as a nucleic acid primer sequence or a template switch oligonucleotide (TSO) sequence.

The support may be a bead. A support such as a solid support, e.g., a bead, may be porous, non-porous, hollow, solid, semi-solid, and/or a combination thereof. Beads may be solid, semi-solid, semi-fluidic, fluidic, and/or a combination thereof. In some instances, a support, e.g., a bead, may be dissolvable, disruptable, and/or degradable. In some cases, a support, e.g., a bead, may not be degradable. In some cases, the support, e.g., a bead, may be a gel bead. A gel bead may be a hydrogel bead. A gel bead may be formed from molecular precursors, such as a polymeric or monomeric species. A semi-solid support, e.g., a bead, may be a liposomal bead. Supports, e.g., beads, may comprise metals including iron oxide, gold, and silver. In some cases, the solid support, e.g., the bead, may be a silica bead. In some cases, the support, e.g., a bead, can be rigid. In other cases, the support, e.g., a bead, may be flexible and/or compressible.

A partition may comprise one or more unique identifiers, such as barcodes. Barcodes may be previously, subsequently or concurrently delivered to the partitions that hold the compartmentalized or partitioned biological particle. For example, barcodes may be injected into droplets before, after, or concurrently with droplet generation. The delivery of the barcodes to a particular partition allows for the later attribution of the characteristics of the individual biological particle to the partition. Barcodes may be delivered, for example on a nucleic acid molecule (e.g., an oligonucleotide), to a partition via any suitable mechanism. Beads are described in further detail herein.

In some cases, barcoded nucleic acid molecules can be initially associated with the bead and then released from the bead. Release of the barcoded nucleic acid molecules can be passive (e.g., by diffusion out of the bead). In addition, or alternatively, release from the bead can be upon application of a stimulus which allows the barcoded nucleic acid nucleic acid molecules to dissociate or to be released from the bead. Such stimulus may disrupt the bead, an interaction that couples the barcoded nucleic acid molecules to or within the bead, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

In some examples, beads, biological particles (or membrane bound particles) and droplets may flow along channels at substantially regular flow profiles (e.g., at regular flow rates). Such regular flow profiles may permit a droplet to include a single bead and a single biological particle. Such regular flow profiles may permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that may be used to provide such regular flow profiles are provided in, for example, U.S. Patent Publication No. 2015/0292988, which is entirely incorporated herein by reference.

Beneficially, a discrete droplet partitioning a biological particle and a barcode carrying bead may effectively allow the attribution of the barcode to macromolecular constituents of the biological particle within the partition. The contents of a partition may remain discrete from the contents of other partitions.

A bead may be porous, non-porous, solid, semi-solid, semi-fluidic, fluidic, and/or a combination thereof. In some instances, a bead may be dissolvable, disruptable, and/or degradable. In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

In some instances, the bead may contain molecular precursors (e.g., monomers or polymers), which may form a polymer network via polymerization of the molecular precursors. In some cases, a precursor may be an already polymerized species capable of undergoing further polymerization via, for example, a chemical cross-linkage. In some cases, a precursor can comprise one or more of an acrylamide or a methacrylamide monomer, oligomer, or polymer. In some cases, the bead may comprise prepolymers, which are oligomers capable of further polymerization. For example, polyurethane beads may be prepared using prepolymers. In some cases, the bead may contain individual polymers that may be further polymerized together. In some cases, beads may be generated via polymerization of different precursors, such that they comprise mixed polymers, co-polymers, and/or block co-polymers. In some cases, the bead may comprise covalent or ionic bonds between polymeric precursors (e.g., monomers, oligomers, linear polymers), nucleic acid molecules (e.g., oligonucleotides), primers, and other entities. In some cases, the covalent bonds can be carbon-carbon bonds, thioether bonds, or carbon-heteroatom bonds.

Cross-linking may be permanent or reversible, depending upon the particular cross-linker used. Reversible cross-linking may allow for the polymer to linearize or dissociate under appropriate conditions. In some cases, reversible cross-linking may also allow for reversible attachment of a material bound to the surface of a bead. In some cases, a cross-linker may form disulfide linkages. In some cases, the chemical cross-linker forming disulfide linkages may be cystamine or a modified cystamine.

In some cases, disulfide linkages can be formed between molecular precursor units (e.g., monomers, oligomers, or linear polymers) or precursors incorporated into a bead and nucleic acid molecules (e.g., oligonucleotides). Cystamine (including modified cystamines), for example, is an organic agent comprising a disulfide bond that may be used as a crosslinker agent between individual monomeric or polymeric precursors of a bead. Polyacrylamide may be polymerized in the presence of cystamine or a species comprising cystamine (e.g., a modified cystamine) to generate polyacrylamide gel beads comprising disulfide linkages (e.g., chemically degradable beads comprising chemically-reducible cross-linkers). The disulfide linkages may permit the bead to be degraded (or dissolved) upon exposure of the bead to a reducing agent.

In some cases, chitosan, a linear polysaccharide polymer, may be crosslinked with glutaraldehyde via hydrophilic chains to form a bead. Crosslinking of chitosan polymers may be achieved by chemical reactions that are initiated by heat, pressure, change in pH, and/or radiation.

In some cases, a bead may comprise an acrydite moiety, which in certain aspects may be used to attach one or more nucleic acid molecules (e.g., barcode sequence, barcoded nucleic acid molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties may be modified to form chemical bonds with a species to be attached, such as a nucleic acid molecule (e.g., barcode sequence, barcoded nucleic acid molecule, barcoded oligonucleotide, primer, or other oligonucleotides). Acrydite moieties may be modified with thiol groups capable of forming a disulfide bond or may be modified with groups already comprising a disulfide bond. The thiol or disulfide (via disulfide exchange) may be used as an anchor point for a species to be attached or another part of the acrydite moiety may be used for attachment. In some cases, attachment can be reversible, such that when the disulfide bond is broken (e.g., in the presence of a reducing agent), the attached species is released from the bead. In other cases, an acrydite moiety can comprise a reactive hydroxyl group that may be used for attachment.

Functionalization of beads for attachment of nucleic acid molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide) that comprises one or more functional sequences, such as a TSO sequence or a primer sequence (e.g., a poly T sequence, or a nucleic acid primer sequence complementary to a target nucleic acid sequence and/or for amplifying a target nucleic acid sequence, a random primer, or a primer sequence for messenger RNA) that is useful for incorporation into the bead, etc.) and/or one or more barcode sequences. The one or more barcode sequences may include sequences that are the same for all nucleic acid molecules coupled to a given bead and/or sequences that are different across all nucleic acid molecules coupled to the given bead. The nucleic acid molecule may be incorporated into the bead.

In some cases, the nucleic acid molecule can comprise one or more functional sequences. For example, a functional sequence can comprise a sequence for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the functional sequence can comprise a barcode sequence or multiple barcode sequences. In some cases, the functional sequence can comprise a unique molecular identifier (UMI). In some cases, the functional sequence can comprise a primer sequence (e.g., an R1 primer sequence for Illumina sequencing, an R2 primer sequence for Illumina sequencing, etc.). In some cases, a functional sequence can comprise a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence may contain a contiguous or continuous portion or segment, but not all, of a full sequence, for example. In some cases, a downstream procedure may extend the partial sequence, or derivative thereof, to achieve a full sequence of the partial sequence, or derivative thereof. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

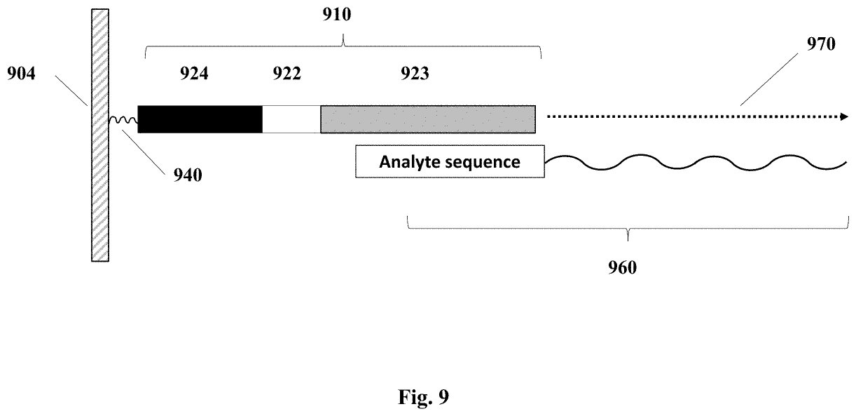

An example barcoded oligonucleotide carried by a bead used in these methods is shown in FIG. 9. In FIG. 9, a barcoded oligonucleotide or capture probe 910 comprises a barcode sequence 922 corresponding to a location on a support 904 to which the oligonucleotide is attached (as illustrated in this example, the oligonucleotide 910 is attached to the support 904 via a modification or chemical moiety 940 capable of attaching to the support 904). The illustrated oligonucleotide 910 also comprises a sequence 923 (i.e., an analyte capture sequence or capture domain) complementary to a sequence of an analyte (e.g., mRNA molecule) 960 from a biological particle (e.g., cell or nucleus). In some instances, sequence 923 comprises a sequence specific for an mRNA molecule. In some instances, sequence 923 comprises a poly(dT) sequence. In some instances, sequence 923 comprises a defined nucleotide sequence, a semi-random nucleotide sequence or a random nucleotide sequence. Sequence 923 is hybridized to mRNA molecule 960 (i.e., the mRNA is captured by the 923 sequence) and extended via a nucleic acid reaction (e.g., a cDNA molecule 970 is generated in a reverse transcription reaction) generating a complementary oligonucleotide comprising barcode sequence 922 (e.g., a spatial barcode sequence, or a reverse complement thereof) and a sequence of the extended nucleic acid (e.g., cDNA 970) (or a portion thereof). A functional sequence 924, such as a primer binding site for amplification and/or a sequencing related primer binding site (e.g., a sequence used for a sequencing reaction), etc. is also included in the barcoded oligonucleotide or capture probe. In some examples, barcoded nucleic acid molecules can then be optionally processed as described elsewhere herein, e.g., to amplify the molecules and/or append sequencing platform specific sequences to the fragments. Barcoded nucleic acid molecules, or derivatives generated therefrom, can then be sequenced on a suitable sequencing platform. Nucleic acid barcode molecule 910 may be attached to support 904 optionally via a releasable linkage 940 (e.g., comprising a labile bond), such as those described in WO2020/047007A2 (Appl. No. PCT/US2019/048430), WO2020/047010A2 (Appl. No. PCT/US2019/048434), WO2020/047004A3 (Appl. No. PCT/US2019/048427), and WO2020/047005A1 (PCT/US2019/048428), each of which are incorporated by reference herein in their entirety.

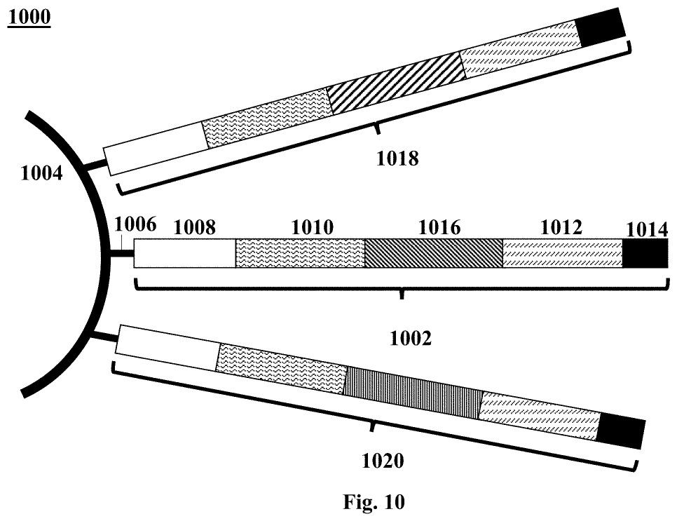

FIG. 10 illustrates an example of a barcode carrying bead. A nucleic acid molecule 1002, such as an oligonucleotide, can be coupled to a bead 1004 by a releasable linkage 1006, such as, for example, a disulfide linker. The same bead 1004 may be coupled (e.g., via releasable linkage) to one or more other nucleic acid molecules 1018, 1020. The nucleic acid molecule 1002 may be or comprise a barcode. As noted elsewhere herein, the structure of the barcode may comprise several sequence elements. The nucleic acid molecule 1002 may comprise a functional sequence 1008 that may be used in subsequent processing. For example, the functional sequence 1008 may include one or more of a sequencer specific flow cell attachment sequence (e.g., a P5 sequence for Illumina® sequencing systems) and a sequencing primer sequence (e.g., a R1 primer for Illumina® sequencing systems), or partial sequence(s) thereof. The nucleic acid molecule 1002 may comprise a barcode sequence 1010 for use in barcoding the sample (e.g., DNA, RNA, protein, etc.). In some cases, the barcode sequence 1010 can be bead-specific such that the barcode sequence 1010 is common to all nucleic acid molecules (e.g., including nucleic acid molecule 1002) coupled to the same bead 1004. Alternatively, or in addition, the barcode sequence 1010 can be partition-specific such that the barcode sequence 1010 is common to all nucleic acid molecules coupled to one or more beads that are partitioned into the same partition. The nucleic acid molecule 1002 may comprise a specific priming sequence 1012, such as an mRNA specific priming sequence (e.g., poly-T sequence), a targeted priming sequence, and/or a random priming sequence. The nucleic acid molecule 1002 may comprise an anchoring sequence 1014 to ensure that the specific priming sequence 1012 hybridizes at the sequence end (e.g., of the mRNA). For example, the anchoring sequence 1014 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 1002 may comprise a unique molecular identifying sequence 1016 (e.g., unique molecular identifier (UMI)). In some cases, the unique molecular identifying sequence 1016 may comprise from about 5 to about 3 nucleotides. Alternatively, the unique molecular identifying sequence 1016 may compress less than about 5 or more than about 8 nucleotides. The unique molecular identifying sequence 1016 may be a unique sequence that varies across individual nucleic acid molecules (e.g., 1002, 1018, 1020, etc.) coupled to a single bead (e.g., bead 1004). In some cases, the unique molecular identifying sequence 1016 may be a random sequence (e.g., such as a random N-mer sequence). For example, the UMI may provide a unique identifier of the starting mRNA molecule that was captured, to allow quantitation of the number of original expressed RNA. As will be appreciated, although FIG. 10 shows three nucleic acid molecules 1002, 1018, 1020 coupled to the surface of the bead 1004, an individual bead may be coupled to any number of individual nucleic acid molecules, for example, from one to tens to hundreds of thousands or even millions of individual nucleic acid molecules. The respective barcodes for the individual nucleic acid molecules can comprise both common sequence segments or relatively common sequence segments (e.g., 1008, 1010, 1012, etc.) and variable or unique sequence segments (e.g., 1016) between different individual nucleic acid molecules coupled to the same bead.

In operation, a biological particle (e.g., cell, nucleus, DNA, RNA, etc.) can be co-partitioned along with a barcode bearing bead 1004. The barcoded nucleic acid molecules 1002, 1018, 1020 can be released from the bead 1004 in the partition. By way of example, in the context of analyzing sample RNA, the poly-T segment (e.g., 1012) of one of the released nucleic acid molecules (e.g., 1002) can hybridize to the poly-A tail of a mRNA molecule. Reverse transcription may result in a cDNA transcript of the mRNA, but which transcript includes each of the sequence segments 1008, 1010, 1016 of the nucleic acid molecule 1002. Because the nucleic acid molecule 1002 comprises an anchoring sequence 1014, it will more likely hybridize to and prime reverse transcription at the sequence end of the poly-A tail of the mRNA. Within any given partition, all the cDNA transcripts of the individual mRNA molecules may include a common barcode sequence segment 1010. However, the transcripts made from the different mRNA molecules within a given partition may vary at the unique molecular identifying sequence 1012 segment (e.g., UMI segment). Beneficially, even following any subsequent amplification of the contents of a given partition, the number of different UMIs can be indicative of the quantity of mRNA originating from a given partition, and thus from the biological particle (e.g., cell or nucleus). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell or nucleus contents.

In some instances, a bead may comprise a capture sequence or binding sequence configured to bind to a corresponding capture sequence or binding sequence. In some instances, a bead may comprise a plurality of different capture sequences or binding sequences configured to bind to different respective corresponding capture sequences or binding sequences. For example, a bead may comprise a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead may comprise any number of different capture sequences. In some instances, a bead may comprise at least 2, 3, 4, 5, 6, 7, 8, 9, 10 or more different capture sequences or binding sequences configured to bind to different respective capture sequences or binding sequences, respectively. Alternatively, or in addition, a bead may comprise at most about 10, 9, 8, 7, 6, 5, 4, 3, or 2 different capture sequences or binding sequences configured to bind to different respective capture sequences or binding sequences. In some instances, the different capture sequences or binding sequences may be configured to facilitate analysis of a same type of analyte. In some instances, the different capture sequences or binding sequences may be configured to facilitate analysis of different types of analytes (with the same bead). The capture sequence may be designed to attach to a corresponding capture sequence. Beneficially, such corresponding capture sequence may be introduced to, or otherwise induced in, a biological particle (e.g., cell, nucleus, cell bead, etc.) for performing different assays in various formats (e.g., barcoded antibodies comprising the corresponding capture sequence, barcoded MHC dextramers comprising the corresponding capture sequence, barcoded guide RNA molecules comprising the corresponding capture sequence, etc.), such that the corresponding capture sequence may later interact with the capture sequence associated with the bead. In some instances, a capture sequence coupled to a bead (or other support) may be configured to attach to a linker molecule, such as a splint molecule, wherein the linker molecule is configured to couple the bead (or other support) to other molecules through the linker molecule, such as to one or more analytes or one or more other linker molecules.

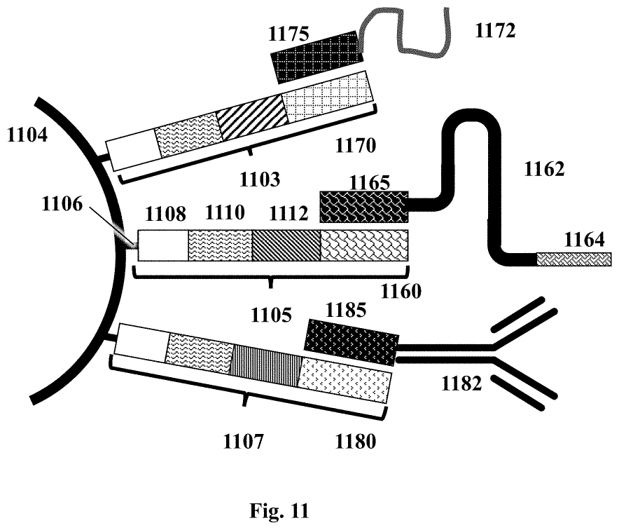

FIG. 11 illustrates another example of a barcode carrying bead. A nucleic acid molecule 1105, such as an oligonucleotide, can be coupled to a bead 1104 by a releasable linkage 1106, such as, for example, a disulfide linker. The nucleic acid molecule 1105 may comprise a first capture sequence 1160. The same bead 1104 may be coupled (e.g., via releasable linkage) to one or more other nucleic acid molecules 1103, 1107 comprising other capture sequences. The nucleic acid molecule 1105 may be or comprise a barcode. As noted elsewhere herein, the structure of the barcode may comprise a number of sequence elements, such as a functional sequence 1108 (e.g., flow cell attachment sequence, sequencing primer sequence, etc.), a barcode sequence 1110 (e.g., bead-specific sequence common to bead, partition-specific sequence common to partition, etc.), and a unique molecular identifier 1112 (e.g., unique sequence within different molecules attached to the bead), or partial sequences thereof. The capture sequence 1160 may be configured to attach to a corresponding capture sequence 1165. In some instances, the corresponding capture sequence 1165 may be coupled to another molecule that may be an analyte or an intermediary carrier. For example, as illustrated in FIG. 11, the corresponding capture sequence 1165 is coupled to a guide RNA molecule 1162 comprising a target sequence 1164, wherein the target sequence 1164 is configured to attach to the analyte. Another oligonucleotide molecule 1107 attached to the bead 1104 comprises a second capture sequence 1180 which is configured to attach to a second corresponding capture sequence 1185. As illustrated in FIG. 11, the second corresponding capture sequence 1185 is coupled to an antibody 1182. In some cases, the antibody 1182 may have binding specificity to an analyte (e.g., surface protein). Alternatively, the antibody 1182 may not have binding specificity. Another oligonucleotide molecule 1103 attached to the bead 1104 comprises a third capture sequence 1170 which is configured to attach to a second corresponding capture sequence 1175. As illustrated in FIG. 11, the third corresponding capture sequence 1175 is coupled to a molecule 1172. The molecule 1172 may or may not be configured to target an analyte. The other oligonucleotide molecules 1103, 1107 may comprise the other sequences (e.g., functional sequence, barcode sequence, UMI, etc.) described with respect to oligonucleotide molecule 1105. While a single oligonucleotide molecule comprising each capture sequence is illustrated in FIG. 11, it will be appreciated that, for each capture sequence, the bead may comprise a set of one or more oligonucleotide molecules each comprising the capture sequence. For example, the bead may comprise any number of sets of one or more different capture sequences. Alternatively, or in addition, the bead 1104 may comprise other capture sequences. Alternatively, or in addition, the bead 1104 may comprise fewer types of capture sequences (e.g., two capture sequences). Alternatively or in addition, the bead 1104 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression.

In operation, the barcoded oligonucleotides may be released (e.g., in a partition), as described elsewhere herein. Alternatively, the nucleic acid molecules bound to the bead (e.g., gel bead) may be used to hybridize and capture analytes (e.g., one or more types of analytes) on the solid phase of the bead.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. For example, some precursors comprising a carboxylic acid (COOH) group can co-polymerize with other precursors to form a gel bead that also comprises a COOH functional group. In some cases, acrylic acid (a species comprising free COOH groups), acrylamide, and bis(acryloyl)cystamine can be co-polymerized together to generate a gel bead comprising free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the bead.

Beads comprising disulfide linkages in their polymeric network may be functionalized with additional species via reduction of some of the disulfide linkages to free thiols. The disulfide linkages may be reduced via, for example, the action of a reducing agent (e.g., DTT, TCEP, etc.) to generate free thiol groups, without dissolution of the bead. Free thiols of the beads can then react with free thiols of a species or a species comprising another disulfide bond (e.g., via thiol-disulfide exchange) such that the species can be linked to the beads (e.g., via a generated disulfide bond). In some cases, free thiols of the beads may react with any other suitable group. For example, free thiols of the beads may react with species comprising an acrydite moiety. The free thiol groups of the beads can react with the acrydite via Michael addition chemistry, such that the species comprising the acrydite is linked to the bead. In some cases, uncontrolled reactions can be prevented by inclusion of a thiol capping agent such as N-ethylmaleimide or iodoacetate.

Activation of disulfide linkages within a bead can be controlled such that only a small number of disulfide linkages are activated. Control may be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent may be used for reduction. Controlling the number of disulfide linkages that are reduced to free thiols may be useful in ensuring bead structural integrity during functionalization. In some cases, optically-active agents, such as fluorescent dyes may be coupled to beads via free thiol groups of the beads and used to quantify the number of free thiols present in a bead and/or track a bead.

In some cases, addition of moieties to a gel bead after gel bead formation may be advantageous. For example, addition of an oligonucleotide (e.g., barcoded oligonucleotide) after gel bead formation may avoid loss of the species during chain transfer termination that can occur during polymerization. Moreover, smaller precursors (e.g., monomers or cross linkers that do not comprise side chain groups and linked moieties) may be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel may possess an upper critical solution temperature (UCST) that can permit temperature driven swelling and collapse of a bead. Such functionality may aid in oligonucleotide (e.g., a primer) infiltration into the bead during subsequent functionalization of the bead with the oligonucleotide. Post-production functionalization may also be useful in controlling loading ratios of species in beads, such that, for example, the variability in loading ratio is minimized. Species loading may also be performed in a batch process such that a plurality of beads can be functionalized with the species in a single batch.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules (e.g., barcoded oligonucleotides), the beads may be degradable, disruptable, or dissolvable spontaneously or upon exposure to one or more stimuli (e.g., temperature changes, pH changes, exposure to particular chemical species or phase, exposure to light, reducing agent, etc.). In some cases, a bead may be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead may be thermally degradable such that when the bead is exposed to an appropriate change in temperature (e.g., heat), the bead degrades. Degradation or dissolution of a bead bound to a species (e.g., a nucleic acid molecule, e.g., barcoded oligonucleotide) may result in release of the species from the bead.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. For example, a polyacrylamide bead comprising cystamine and linked, via a disulfide bond, to a barcode sequence, may be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet comprising a bead-bound barcode sequence in basic solution may also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing nucleic acid molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

In some cases, an acrydite moiety linked to a precursor, another species linked to a precursor, or a precursor itself can comprise a labile bond, such as chemically, thermally, or photo-sensitive bond e.g., disulfide bond, UV sensitive bond, or the like. Once acrydite moieties or other moieties comprising a labile bond are incorporated into a bead, the bead may also comprise the labile bond. The labile bond may be, for example, useful in reversibly linking (e.g., covalently linking) species (e.g., barcodes, primers, etc.) to a bead. In some cases, a thermally labile bond may include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the support (e.g., bead).

The addition of multiple types of labile bonds to a gel bead may result in the generation of a bead capable of responding to varied stimuli. Each type of labile bond may be sensitive to an associated stimulus (e.g., chemical stimulus, light, temperature, enzymatic, etc.) such that release of species attached to a bead via each labile bond may be controlled by the application of the appropriate stimulus. Such functionality may be useful in controlled release of species from a gel bead. In some cases, another species comprising a labile bond may be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

In some cases, a species (e.g., oligonucleotide molecules comprising barcodes) that are attached to a solid support (e.g., a bead) may comprise a U-excising element that allows the species to release from the bead. In some cases, the U-excising element may comprise a single-stranded DNA (ssDNA) sequence that contains at least one uracil. The species may be attached to a solid support via the ssDNA sequence containing the at least one uracil. The species may be released by a combination of uracil-DNA glycosylase (e.g., to remove the uracil) and an endonuclease (e.g., to induce an ssDNA break). If the endonuclease generates a 5′ phosphate group from the cleavage, then additional enzyme treatment may be included in downstream processing to eliminate the phosphate group, e.g., prior to ligation of additional sequencing handle elements, e.g., Illumina full P5 sequence, partial P5 sequence, full R1 sequence, and/or partial R1 sequence.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that may be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNase). A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. Such species may be entered into polymerization reaction mixtures such that generated beads comprise the species upon bead formation. In some cases, such species may be added to the gel beads after formation. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNase, etc. Such species may include one or more reagents described elsewhere herein (e.g., lysis agents, inhibitors, inactivating agents, chelating agents, stimulus). Trapping of such species may be controlled by the polymer network density generated during polymerization of precursors, control of ionic charge within the gel bead (e.g., via ionic species linked to polymerized species), or by the release of other species. Encapsulated species may be released from a bead upon bead degradation and/or by application of a stimulus capable of releasing the species from the bead. Alternatively, or in addition, species may be partitioned in a partition (e.g., droplet) during or after partition formation. Such species may include, without limitation, the abovementioned species that may also be encapsulated in a bead.

A degradable bead may comprise one or more species with a labile bond such that, when the bead/species is exposed to the appropriate stimuli, the bond is broken, and the bead degrades. The labile bond may be a chemical bond (e.g., covalent bond, ionic bond) or may be another type of physical interaction (e.g., van der Waals interactions, dipole-dipole interactions, etc.). In some cases, a crosslinker used to generate a bead may comprise a labile bond. Upon exposure to the appropriate conditions, the labile bond can be broken, and the bead degraded. For example, upon exposure of a polyacrylamide gel bead comprising cystamine crosslinkers to a reducing agent, the disulfide bonds of the cystamine can be broken and the bead degraded.

A degradable bead may be useful in more quickly releasing an attached species (e.g., a nucleic acid molecule, a barcode sequence, a primer, etc.) from the bead when the appropriate stimulus is applied to the bead as compared to a bead that does not degrade. For example, for a species bound to an inner surface of a porous bead or in the case of an encapsulated species, the species may have greater mobility and accessibility to other species in solution upon degradation of the bead. In some cases, a species may also be attached to a degradable bead via a degradable linker (e.g., disulfide linker). The degradable linker may respond to the same stimuli as the degradable bead or the two degradable species may respond to different stimuli. For example, a barcode sequence may be attached, via a disulfide bond, to a polyacrylamide bead comprising cystamine. Upon exposure of the barcoded bead to a reducing agent, the bead degrades, and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation may refer to the disassociation of a bound or entrained species from a bead, both with and without structurally degrading the physical bead itself. For example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

Where degradable beads are provided, it may be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads comprise reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it may be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that may be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers may be used to trigger the degradation of beads. Examples of these chemical changes may include but are not limited to pH-mediated changes to the integrity of a component within the bead, degradation of a component of a bead via cleavage of cross-linked bonds, and depolymerization of a component of a bead.

In some embodiments, a bead may be formed from materials that comprise degradable chemical crosslinkers, such as BAC or cystamine. Degradation of such degradable crosslinkers may be accomplished through several mechanisms. In some examples, a bead may be contacted with a chemical degrading agent that may induce oxidation, reduction, or other chemical changes. For example, a chemical degrading agent may be a reducing agent, such as dithiothreitol (DTT). Additional examples of reducing agents may include β-mercaptoethanol, (2S)-2-amino-1,4-dimercaptobutane (dithiobutylamine or DTBA), tris(2-carboxyethyl) phosphine (TCEP), or combinations thereof. A reducing agent may degrade the disulfide bonds formed between gel precursors forming the bead, and thus, degrade the bead. In other cases, a change in pH of a solution, such as an increase in pH, may trigger degradation of a bead. In other cases, exposure to an aqueous solution, such as water, may trigger hydrolytic degradation, and thus degradation of the bead. In some cases, any combination of stimuli may trigger degradation of a bead. For example, a change in pH may enable a chemical agent (e.g., DTT) to become an effective reducing agent.

Beads may also be induced to release their contents upon the application of a thermal stimulus. A change in temperature can cause a variety of changes to a bead. For example, heat can cause a solid bead to liquefy. A change in heat may cause melting of a bead such that a portion of the bead degrades. In other cases, heat may increase the internal pressure of the bead components such that the bead ruptures or explodes. Heat may also act upon heat-sensitive polymers used as materials to construct beads.

Any suitable agent may degrade beads. In some embodiments, changes in temperature or pH may be used to degrade thermo-sensitive or pH-sensitive bonds within beads. In some embodiments, chemical degrading agents may be used to degrade chemical bonds within beads by oxidation, reduction, or other chemical changes. For example, a chemical degrading agent may be a reducing agent, such as DTT, wherein DTT may degrade the disulfide bonds formed between a crosslinker and gel precursors, thus degrading the bead. In some embodiments, a reducing agent may be added to degrade the bead, which may or may not cause the bead to release its contents. Examples of reducing agents may include dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof. The reducing agent may be present at a concentration of about 0.1 mM, 0.5 mM, 1 mM, 5 mM, 10 mM. The reducing agent may be present at a concentration of at least about 0.1 mM, 0.5 mM, 1 mM, 5 mM, 10 mM, or greater than 10 mM. The reducing agent may be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

Although FIG. 3 and FIG. 6 have been described in terms of providing substantially singly occupied partitions, above, in certain cases, it may be desirable to provide multiply occupied partitions, e.g., containing two, three, four or more cells or nuclei and/or supports (e.g., beads) comprising barcoded nucleic acid molecules (e.g., oligonucleotides) within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

In some cases, additional supports (e.g., beads) can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction (e.g., junction 310). In such cases, the flow and frequency of the different beads into the channel or junction may be controlled to provide for a certain ratio of beads from each source, while ensuring a given pairing or combination of such beads into a partition with a given number of biological particles (e.g., one biological particle and one bead per partition).

The partitions described herein may comprise small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with supports (e.g., beads), it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions. In some examples, any of the methods disclosed herein may be distinguished by having the advantage of high throughput processing of fixed cells in partitions (e.g., droplets in an emulsion or wells, such as microwells). As further described herein, the methods of the present disclosure allow for thousands to tens of thousands to hundreds of thousands fixed cells or nuclei to be analyzed on a single cell level in individual partitions containing individual cells or nuclei.

### Reagents

In accordance with certain aspects, biological particles may be partitioned along with lysis reagents to release the contents of the biological particles within the partition. In such cases, the lysis agents can be contacted with the biological particle suspension concurrently with, or immediately prior to, the introduction of the biological particles into the partitioning junction/droplet generation zone (e.g., junction 310 in FIG. 3), such as through an additional channel or channels upstream of the channel junction. In accordance with other aspects, additionally or alternatively, biological particles may be partitioned along with other reagents, as will be described further below.

Beneficially, when lysis reagents and biological particles are co-partitioned, the lysis reagents can facilitate the release of the contents of the biological particles within the partition. The contents released in a partition may remain discrete from the contents of other partitions.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysis agents may additionally or alternatively be co-partitioned with the biological particles to cause the release of the biological particle's contents into the partitions. For example, in some cases, surfactant-based lysis solutions may be used to lyse cells, although these may be less desirable for emulsion-based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of biological particles that may be in addition to or in place of droplet partitioning, where any pore size of the encapsulate is sufficiently small to retain nucleic acid fragments of a given size, following cellular disruption.

Alternatively or in addition to the lysis agents co-partitioned with the analyte carriers described above, other reagents can also be co-partitioned with the analyte carriers, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated analyte carriers (e.g., a cell or a nucleus in a polymer matrix), the analyte carriers may be exposed to an appropriate stimulus to release the analyte carriers or their contents from a co-partitioned support (e.g., bead). For example, in some cases, a chemical stimulus may be co-partitioned along with an encapsulated analyte carrier to allow for the degradation of the support (e.g., bead) and release of the cell, nucleus or its contents into the larger partition. In some cases, this stimulus may be the same as the stimulus described elsewhere herein for release of nucleic acid molecules (e.g., oligonucleotides) from their respective support (e.g., bead). In alternative examples, this may be a different and non-overlapping stimulus, to allow an encapsulated analyte carrier to be released into a partition at a different time from the release of nucleic acid molecules into the same partition. For a description of methods, compositions, and systems for encapsulating cells (also referred to as a “cell bead”), see, e.g., U.S. Pat. No. 10,428,326 and U.S. Pat. Pub. 20190100632, which are each incorporated by reference in their entirety.

Additional reagents may also be co-partitioned with the biological particles, such as endonucleases to fragment a biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNase, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may comprise a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA.

In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo may be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo may be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells or nuclei are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle or groups of biological particles with the unique identifiers, such as described above (with reference to FIGS. 3 and 6). In some aspects, the unique identifiers are provided in the form of nucleic acid molecules (e.g., oligonucleotides) that comprise nucleic acid barcode sequences that may be attached to or otherwise associated with the nucleic acid contents of individual biological particle, or to other components of the biological particle, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying nucleic acids (e.g., mRNA, the genomic DNA) from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides (e.g., attached to a bead) into partitions, e.g., droplets within microfluidic systems.

In an example, supports, such as beads, are provided that each include large numbers of the above described barcoded nucleic acid molecules (e.g., barcoded oligonucleotides) releasably attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid molecules into the partitions, as they are capable of carrying large numbers of nucleic acid molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, it may be desirable to incorporate multiple different barcodes within a given partition, either attached to single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles and may be degraded for release of the attached nucleic acid molecules through exposure to a reducing agent, such as DTT.

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size. For example, regarding FIG. 6, a discrete droplet generated may include a bead (e.g., as in occupied droplets 616). Alternatively, a discrete droplet generated may include more than one bead. Alternatively, a discrete droplet generated may not include any beads (e.g., as in unoccupied droplet 618). In some instances, a discrete droplet generated may contain one or more biological particles, as described elsewhere herein. In some instances, a discrete droplet generated may comprise one or more reagents, as described elsewhere herein.

In some instances, the aqueous fluid 608 can have a substantially uniform concentration or frequency of beads 612. The beads 612 can be introduced into the channel segment 602 from a separate channel (not shown in FIG. 6). The frequency of beads 612 in the channel segment 602 may be controlled by controlling the frequency in which the beads 612 are introduced into the channel segment 602 and/or the relative flow rates of the fluids in the channel segment 602 and the separate channel. In some instances, the beads can be introduced into the channel segment 602 from a plurality of different channels, and the frequency controlled accordingly.

In some instances, the aqueous fluid 608 in the channel segment 602 can comprise biological particles (e.g., described with reference to FIGS. 3 and 6). In some instances, the aqueous fluid 608 can have a substantially uniform concentration or frequency of biological particles. As with the beads, the biological particles can be introduced into the channel segment 602 from a separate channel. The frequency or concentration of the biological particles in the aqueous fluid 608 in the channel segment 602 may be controlled by controlling the frequency in which the biological particles are introduced into the channel segment 602 and/or the relative flow rates of the fluids in the channel segment 602 and the separate channel. In some instances, the biological particles can be introduced into the channel segment 602 from a plurality of different channels, and the frequency controlled accordingly. In some instances, a first separate channel can introduce beads and a second separate channel can introduce biological particles into the channel segment 602. The first separate channel introducing the beads may be upstream or downstream of the second separate channel introducing the biological particles.

The second fluid 610 can comprise an oil, such as a fluorinated oil, that includes a fluorosurfactant for stabilizing the resulting droplets, for example, inhibiting subsequent coalescence of the resulting droplets.

In some instances, the second fluid 610 may not be subjected to and/or directed to any flow in or out of the reservoir 604. For example, the second fluid 610 may be substantially stationary in the reservoir 604. In some instances, the second fluid 610 may be subjected to flow within the reservoir 604, but not in or out of the reservoir 604, such as via application of pressure to the reservoir 604 and/or as affected by the incoming flow of the aqueous fluid 608 at the junction 606. Alternatively, the second fluid 610 may be subjected and/or directed to flow in or out of the reservoir 604. For example, the reservoir 604 can be a channel directing the second fluid 610 from upstream to downstream, transporting the generated droplets.

The channel structure 600 at or near the junction 606 may have certain geometric features that at least partly determine the sizes of the droplets formed by the channel structure 600. The channel segment 602 can have a height, h₀ and width, w, at or near the junction 606. By way of example, the channel segment 602 can comprise a rectangular cross-section that leads to a reservoir 604 having a wider cross-section (such as in width or diameter). Alternatively, the cross-section of the channel segment 602 can be other shapes, such as a circular shape, trapezoidal shape, polygonal shape, or any other shapes. The top and bottom walls of the reservoir 604 at or near the junction 606 can be inclined at an expansion angle, a. The expansion angle, a, allows the tongue (portion of the aqueous fluid 608 leaving channel segment 602 at junction 606 and entering the reservoir 604 before droplet formation) to increase in depth and facilitate decrease in curvature of the intermediately formed droplet. Droplet size may decrease with increasing expansion angle. The resulting droplet radius, Rd, may be predicted by the following equation for the aforementioned geometric parameters of h₀, w, and a:

$${R}_{d} \approx 0.44 ( 1 + 2.2 \sqrt{tan α} \frac{w}{{h}_{0}} ) \frac{{h}_{0}}{\sqrt{tan α}}$$

By way of example, for a channel structure with w=21 μm, h=21 μm, and α=3°, the predicted droplet size is 121 μm. In another example, for a channel structure with w=25 h=25 μm, and α=5°, the predicted droplet size is 123 μm. In another example, for a channel structure with w=28 μm, h=28 μm, and α=7°, the predicted droplet size is 124 μm.

In some instances, the expansion angle, a, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, or less. In some instances, the width, w, can be between a range of from about 100 micrometers (m) to about 500 μm. In some instances, the width, w, can be between a range of from about 10 μm to about 200 μm. Alternatively, the width can be less than about 10 μm. Alternatively, the width can be greater than about 500 μm. In some instances, the flow rate of the aqueous fluid 608 entering the junction 606 can be between about 0.04 microliters (μL)/minute (min) and about 40 μL/min. In some instances, the flow rate of the aqueous fluid 608 entering the junction 606 can be between about 0.01 microliters (μL)/minute (min) and about 100 μL/min. Alternatively, the flow rate of the aqueous fluid 608 entering the junction 606 can be less than about 0.01 μL/min. Alternatively, the flow rate of the aqueous fluid 608 entering the junction 606 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 608 entering the junction 606.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 606) between aqueous fluid 608 channel segments (e.g., channel segment 602) and the reservoir 604. Alternatively, or in addition, the throughput of droplet generation can be increased by increasing the flow rate of the aqueous fluid 608 in the channel segment 602.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The channel networks, e.g., as described above or elsewhere herein, can be fluidly coupled to appropriate fluidic components. For example, the inlet channel segments are fluidly coupled to appropriate sources of the materials they are to deliver to a channel junction. These sources may include any of a variety of different fluidic components, from simple reservoirs defined in or connected to a body structure of a microfluidic device, to fluid conduits that deliver fluids from off-device sources, manifolds, fluid flow units (e.g., actuators, pumps, compressors) or the like. Likewise, the outlet channel segment (e.g., channel segment 608, reservoir 604, etc.) may be fluidly coupled to a receiving vessel or conduit for the partitioned cells or nuclei for subsequent processing. Again, this may be a reservoir defined in the body of a microfluidic device, or it may be a fluidic conduit for delivering the partitioned cells or nuclei to a subsequent process operation, instrument, or component.

The methods and systems described herein may be used to greatly increase the efficiency of single cell or nucleus applications and/or other applications receiving droplet-based input. For example, following the sorting of occupied cells and/or appropriately-sized cells, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken, and the contents of the droplet pooled for additional operations. Additional reagents that may be co-partitioned along with the barcode bearing bead may include oligonucleotides to block ribosomal RNA (rRNA) and nucleases to digest genomic DNA from cells or nuclei. Alternatively, rRNA removal agents may be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, may be subject to sequencing for sequence analysis. In some cases, amplification may be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

### Enzymes, Polymerases and Reverse Transcriptases

In some examples, various assays are performed on un-fixed cells, nuclei and/or biomolecules from un-fixed cells or nuclei. Exemplary assays may include single-cell transcription profiling, single-cell sequence analysis, and the like. These and other assays may be carried out using available systems for encapsulating biological samples, gel beads, barcodes, and/or other compounds/materials in droplets, such as The Chromium System (10× Genomics, Pleasanton, Calif., USA).

Example reagents used in these assays may include a variety of enzymes, including polymerases, such as DNA or RNA polymerases, for example. The enzymes used in these assays may be isolated enzymes and/or purified enzymes.

Example assay reagents may include reverse transcriptases (RTs). RT refers to an enzyme that can synthesize single-stranded DNA using an RNA template (e.g., first-strand synthesis). Generally, RT enzymes may also synthesize single-stranded DNA using a DNA template (e.g., second-strand synthesis).

In some examples, RT may have terminal transferase activity. RT enzymes may have template switching activity, which generally relies on ability of an RT having terminal transferase activity to add non-templated deoxynucleotides to the 3′ end of a DNA strand synthesized in first-strand synthesis. In presence of a template-switching oligonucleotide that can hybridize to the non-templated sequence added to the 3′ end of the first stand, a template-switching reverse transcriptase may “switch” from using the RNA template that serves as the template for first-strand synthesis, to using the template-switching oligonucleotide as template, to extend the length of the first-strand synthesis product.

In some embodiments, template switching can be used to increase the length of cDNA generated in an assay. In some embodiments, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner.

In some examples, RT may have RNase H activity.

Reverse transcriptases may come from a variety of different systems. In some examples, reverse transcriptases may be from, may be derived from, or may be variants of Avian myeloblastosis virus (AMV) RT or Moloney Murine leukemia virus (MMLV) RT.

Additional assay reagents that may be used with RT enzymes may include RNA or DNA templates, primers for first-stand or second-strand DNA synthesis, and template switching oligonucleotides. Reverse transcriptase enzymes generally require including deoxyribonucleotides (dNTPs). Reverse transcriptase enzymes may require magnesium ions (Mg2+) and/or manganese ions (Mn2+) for activity.

Additional example assay reagents that may be used to perform assays using un-fixed cells or nuclei and/or biomolecules include any assay reagents that can be used to perform one or more chemical or biochemical operations encapsulated in a droplet. Accordingly, assay reagents useful in the assay method include any reagents useful in performing a reaction such as nucleic acid modification (e.g., ligation, digestion, methylation, random mutagenesis, bisulfite conversion, uracil hydrolysis, nucleic acid repair, capping, or decapping), nucleic acid amplification (e.g., isothermal amplification or PCR), nucleic acid insertion or cleavage (e.g., via CRISPR/Cas9-mediated or transposon-mediated insertion or cleavage), and/or reverse transcription. Additionally, useful assay reagents can include those that allow the preparation of a target sequence or sequencing reads that are specific to the macromolecular constituents of interest at a higher rate than to non-target sequence specific reads.

### Polyethylene Glycol (PEG)

In some examples, various polymers may provide benefits to un-fixing of fixed cells or nuclei. In some examples, we have found that addition of polyethylene glycol (PEG) to various un-fixing reactions increases the amount and quality of RNA retrieved from the unfixed cells or nuclei. Increased quality of RNA may include lengths of retrieved RNA, the ability of the retrieved RNA to be used as a template in various enzymatic reactions, the sequence complexity of DNA libraries made using a population of retrieved RNA, and the like.

In some examples, various polymers may provide benefits to assays performed on or using un-fixed cells, nuclei or biomolecules obtained from un-fixed cells or nuclei. In some examples, polyethylene glycol (PEG) increases activities of various enzymes. In some examples, PEG increases activities of a first enzyme in the presence of a second enzyme or substance where, without the presence of PEG, the second enzyme or substance would decrease the activity of the first enzyme. In some examples, we have found that addition of PEG to reverse transcription reactions, where the reactions may also contain components from un-fixing reactions (e.g., proteases, other un-fixing agents), increases various activities of reverse transcriptase where, in absence of PEG, the components from the un-fixing reactions would inhibit or destroy reverse transcriptase activities.

In some examples, a beneficial polymer that provides these effects includes polyethylene glycol (PEG), also sometimes referred to as polyethylene oxide (PEO) or polyoxyethylene (POE). PEG is an oligomer or polymer of ethylene oxide. PEG generally refers to polyether compounds with the chemical formula H—(O—CH₂—CH₂)ₙ—OH. PEG is generally prepared by polymerization of ethylene oxide and is available in a wide range of molecular weights. In some examples, PEG is available in different molecular weights from 300 g/mol to 10,000,000 g/mol. Commercially available PEG may be labeled, for example, as PEG 6000 or PEG 8000. These labeling designations generally mean that the average molecular weight of the polymer molecules is 6000 or 8000, respectively. These compositions are generally polydisperse (i.e., they have a distribution of molecular weights). Lower molecular weight PEGs may be available as pure oligomers, referred to as monodisperse, uniform or discrete. Different forms of PEG are available based on the initiator used in the polymerization used to make the substance. PEGs are also available with different geometries, including branched PEGs, Star PEGS and Comb PEGS. Herein, all types of PEG may be useful in methods disclosed herein.

In some examples, an average molecular weight of PEG used in the methods disclosed here may be 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, 12000, 12100, 12200, 12300, 12400, 12500, 12600, 12700, 12800, 12900, 13000, 13100, 13200, 13300, 13400, 13500, 13600, 13700, 13800, 13900, 14000, 14100, 14200, 14300, 14400, 14500, 14600, 14700, 14800, 14900, 15000, 15100, 15200, 15300, 15400, 15500, 15600, 15700, 15800, 15900, 16000, 16100, 16200, 16300, 16400, 16500, 16600, 16700, 16800, 16900, 17000, 17100, 17200, 17300, 17400, 17500, 17600, 17700, 17800, 17900, 18000, 18100, 18200, 18300, 18400, 18500, 18600, 18700, 18800, 18900, 19000, 19100, 19200, 19300, 19400, 19500, 19600, 19700, 19800, 19900, 20000, 20100, 20200, 20300, 20400, 20500, 20600, 20700, 20800, 20900, 21000, 21100, 21200, 21300, 21400, 21500, 21600, 21700, 21800, 21900, 22000, or more. In some examples, the average molecular weight of PEG used in the disclosed methods, compositions, and so forth, may be in a range between any two of the numbers above.

In some examples, an average final concentration of PEG used in the methods disclosed here may be 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, 25, 25.1, 25.2, 25.3, 25.4, 25.5, 25.6, 25.7, 25.8, 25.9, 26, 26.1, 26.2, 26.3, 26.4, 26.5, 26.6, 26.7, 26.8, 26.9, 27, 27.1, 27.2, 27.3, 27.4, 27.5, 27.6, 27.7, 27.8, 27.9, 28, 28.1, 28.2, 28.3, 28.4, 28.5, 28.6, 28.7, 28.8, 28.9, 29, 29.1, 29.2, 29.3, 29.4, 29.5, 29.6, 29.7, 29.8, 29.9, 30, 30.1, 30.2, 30.3, 30.4, 30.5, 30.6, 30.7, 30.8, 30.9, 31, 31.1, 31.2, 31.3, 31.4, 31.5, 31.6, 31.7, 31.8, 31.9, 32, 32.1, 32.2, 32.3, 32.4, 32.5, 32.6, 32.7, 32.8, 32.9, 33, 33.1, 33.2, 33.3, 33.4, 33.5, 33.6, 33.7, 33.8, 33.9, 34, 34.1, 34.2, 34.3, 34.4, 34.5, 34.6, 34.7, 34.8, 34.9, 35, 35.1, 35.2, 35.3, 35.4, 35.5, 35.6, 35.7, 35.8, 35.9, 36, 36.1, 36.2, 36.3, 36.4, 36.5, 36.6, 36.7, 36.8, 36.9, 37, 37.1, 37.2, 37.3, 37.4, 37.5, 37.6, 37.7, 37.8, 37.9, 38, 38.1, 38.2, 38.3, 38.4, 38.5, 38.6, 38.7, 38.8, 38.9, 39, 39.1, 39.2, 39.3, 39.4, 39.5, 39.6, 39.7, 39.8, 39.9, 40, 40.1, 40.2, 40.3, 40.4, 40.5, 40.6, 40.7, 40.8, 40.9, 41, 41.1, 41.2, 41.3, 41.4, 41.5, 41.6, 41.7, 41.8, 41.9, 42, 42.1, 42.2, 42.3, 42.4, 42.5, 42.6, 42.7, 42.8, 42.9, 43, 43.1, 43.2, 43.3, 43.4, 43.5, 43.6, 43.7, 43.8, 43.9, 44, 44.1, 44.2, 44.3, 44.4, 44.5, 44.6, 44.7, 44.8, 44.9, 45, 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, 48, 48.1, 48.2, 48.3, 48.4, 48.5, 48.6, 48.7, 48.8, 48.9, 49, 49.1, 49.2, 49.3, 49.4, 49.5, 49.6, 49.7, 49.8, 49.9, 50, 50.1, 50.2, 50.3, 50.4, 50.5, 50.6, 50.7, 50.8, 50.9, 51, 51.1, 51.2, 51.3, 51.4, 51.5, 51.6, 51.7, 51.8, 51.9, 52, 52.1, 52.2, 52.3, 52.4, 52.5, 52.6, 52.7, 52.8, 52.9, 53, 53.1, 53.2, 53.3, 53.4, 53.5, 53.6, 53.7, 53.8, 53.9, 54, 54.1, 54.2, 54.3, 54.4, 54.5, 54.6, 54.7, 54.8, 54.9, 55, 55.1, 55.2, 55.3, 55.4, 55.5, 55.6, 55.7, 55.8, 55.9, 56, 56.1, 56.2, 56.3, 56.4, 56.5, 56.6, 56.7, 56.8, 56.9, 57, 57.1, 57.2, 57.3, 57.4, 57.5, 57.6, 57.7, 57.8, 57.9, 58, 58.1, 58.2, 58.3, 58.4, 58.5, 58.6, 58.7, 58.8, 58.9, 59, 59.1, 59.2, 59.3, 59.4, 59.5, 59.6, 59.7, 59.8, 59.9, 60, 60.1, 60.2, 60.3, 60.4, 60.5, 60.6, 60.7, 60.8, 60.9, 61, 61.1, 61.2, 61.3, 61.4, 61.5, 61.6, 61.7, 61.8, 61.9, 62, 62.1, 62.2, 62.3, 62.4, 62.5, 62.6, 62.7, 62.8, 62.9, 63, 63.1, 63.2, 63.3, 63.4, 63.5, 63.6, 63.7, 63.8, 63.9, 64, 64.1, 64.2, 64.3, 64.4, 64.5, 64.6, 64.7, 64.8, 64.9, or 65 percent. In some examples, the average final concentration of PEG in the disclosed methods, compositions, and so forth, may be in a range between any two of the numbers above.

In some examples, PEG is dissolved in an aqueous solution and added to a reaction to achieve a desired final concentration. Instead, PEG may be attached to a protein or enzyme, processes called PEGylation. Such processes are well known in the art. A reverse transcriptase (RT), for example, that has attached PEG may be called PEGylated RT.

### Microwell-Based Analysis

As described herein, one or more processes can be performed in a partition, which can be a well. The well can be a well of a plurality of wells of a substrate, such as a microwell of a microwell array or plate, or the well can be a microwell or microchamber of a device (e.g., microfluidic device) comprising a substrate. The well can be a well of a well array or plate, or the well can be a well or chamber of a device (e.g., fluidic device). Accordingly, the wells or microwells can assume an “open” configuration, in which the wells or microwells are exposed to the environment (e.g., contain an open surface) and are accessible on one planar face of the substrate, or the wells or microwells can assume a “closed” or “sealed” configuration, in which the microwells are not accessible on a planar face of the substrate. In some instances, the wells or microwells can be configured to toggle between “open” and “closed” configurations. For instance, an “open” microwell or set of microwells can be “closed” or “sealed” using a membrane (e.g., semi-permeable membrane), an oil (e.g., fluorinated oil to cover an aqueous solution), or a lid, as described elsewhere herein. The wells or microwells can be initially provided in a “closed” or “sealed” configuration, wherein they are not accessible on a planar surface of the substrate without an external force. For instance, the “closed” or “sealed” configuration can include a substrate such as a sealing film or foil that is puncturable or pierceable by pipette tip(s). Suitable materials for the substrate include, without limitation, polyester, polypropylene, polyethylene, vinyl, and aluminum foil.

In some embodiments, the well can have a volume of less than 1 milliliter (mL). For example, the well can be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well can be configured to hold a volume of about 1000 μL, about 100 μL, about 10 μL, about 1 μL, about 100 nL, about 10 nL, about 1 nL, about 100 pL, about 10 pL, etc. The well can be configured to hold a volume of at least 10 pL, at least 100 pL, at least 1 nL, at least 10 nL, at least 100 nL, at least 1 μL, at least 10 μL, at least 100 μL, at least 1000 μL, or more. The well can be configured to hold a volume in a range of volumes listed herein, for example, from about 5 nL to about 20 nL, from about 1 nL to about 100 nL, from about 500 pL to about 100 μL, etc. The well can be of a plurality of wells that have varying volumes and can be configured to hold a volume appropriate to accommodate any of the partition volumes described herein.

In some instances, a microwell array or plate includes a single variety of microwells. In some instances, a microwell array or plate includes a variety of microwells. For instance, the microwell array or plate can include one or more types of microwells within a single microwell array or plate. The types of microwells can have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate can include any number of different types of microwells. For example, the microwell array or plate can include 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well can have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate includes different types of microwells that are located adjacent to one another within the array or plate. For example, a microwell with one set of dimensions can be located adjacent to and in contact with another microwell with a different set of dimensions. Similarly, microwells of different geometries can be placed adjacent to or in contact with one another. The adjacent microwells can be configured to hold different articles; for example, one microwell can be used to contain a biological particle, such as a cell, a nucleus, or other sample (e.g., cellular components, nucleic acid molecules, etc.) while the adjacent microwell can be used to contain a support (e.g., a bead such as a gel bead), droplet, or other reagent. In some cases, the adjacent microwells can be configured to merge the contents held within, e.g., upon application of a stimulus, or spontaneously, upon contact of the articles in each microwell.

As is described elsewhere herein, a plurality of partitions can be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells can include both unoccupied wells (e.g., empty wells) and occupied wells.

A well can include any of the reagents described herein, or combinations thereof. These reagents can include, for example, barcode molecules, enzymes, adapters, and combinations thereof. The reagents can be physically separated from a biological particle (for example, a cell, a nucleus, or cellular components, e.g., proteins, nucleic acid molecules, etc.) that is placed in the well. This physical separation can be accomplished by containing the reagents within, or coupling to, a support (e.g., a bead such as a gel bead) that is placed within a well. The physical separation can also be accomplished by dispensing the reagents in the well and overlaying the reagents with a layer that is, for example, dissolvable, meltable, or permeable prior to introducing the polynucleotide sample into the well. This layer can be, for example, an oil, wax, membrane (e.g., semi-permeable membrane), or the like. The well can be sealed at any point, for example, after addition of the support or bead, after addition of the reagents, or after addition of either of these components. The sealing of the well can be useful for a variety of purposes, including preventing escape of beads or loaded reagents from the well, permitting select delivery of certain reagents (e.g., via the use of a semi-permeable membrane), for storage of the well prior to or following further processing, etc.

A well can include free reagents and/or reagents encapsulated in, or otherwise coupled to or associated with, supports (e.g., beads), or droplets. In some embodiments, any of the reagents described in this disclosure can be encapsulated in, or otherwise coupled to, a support (e.g., a bead) or a droplet, with any chemicals, particles, and elements suitable for sample processing reactions involving biomolecules, such as, but not limited to, nucleic acid molecules and proteins. For example, a bead or droplet used in a sample preparation reaction for DNA sequencing can include one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, oligonucleotides, nucleotides, deoxyribonucleotide triphosphates (dNTPs), dideoxyribonucleotide triphosphates (ddNTPs), DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA, polymerase, ligase, restriction enzymes, proteases, nucleases, protease inhibitors, nuclease inhibitors, chelating agents, reducing agents, oxidizing agents, fluorophores, probes, chromophores, dyes, organics, emulsifiers, surfactants, stabilizers, polymers, water, small molecules, pharmaceuticals, radioactive molecules, preservatives, antibiotics, aptamers, and pharmaceutical drug compounds. As described herein, one or more reagents in the well can be used to perform one or more reactions, including but not limited to: biological particle (e.g., a cell or a nucleus) processing such as lysis, fixation, permeabilization, nucleic acid reactions, e.g., nucleic acid extension reactions, amplification, reverse transcription, reactions, etc.

The wells disclosed herein can be provided as a part of a kit. For example, a kit can include instructions for use, a microwell array or device, and reagents (e.g., beads). The kit can include any useful reagents for performing the processes described herein, e.g., nucleic acid reactions, barcoding of nucleic acid molecules, sample processing (e.g., for biological particle lysis, fixation, and/or permeabilization).

In some cases, a well includes a support (e.g., a bead) or droplet that includes a set of reagents that has a similar attribute, for example, a set of enzymes, a set of minerals, a set of oligonucleotides, a mixture of different barcode molecules, or a mixture of identical barcode molecules. In other cases, a support (e.g., a bead) or droplet includes a heterogeneous mixture of reagents. In some cases, the heterogeneous mixture of reagents can include all components necessary to perform a reaction. In some cases, such mixture can include all components necessary to perform a reaction, except for 1, 2, 3, 4, 5, or more components necessary to perform a reaction. In some cases, such additional components are contained within, or otherwise coupled to, a different support (e.g., a bead) or droplet, or within a solution within a partition (e.g., microwell) of the system.

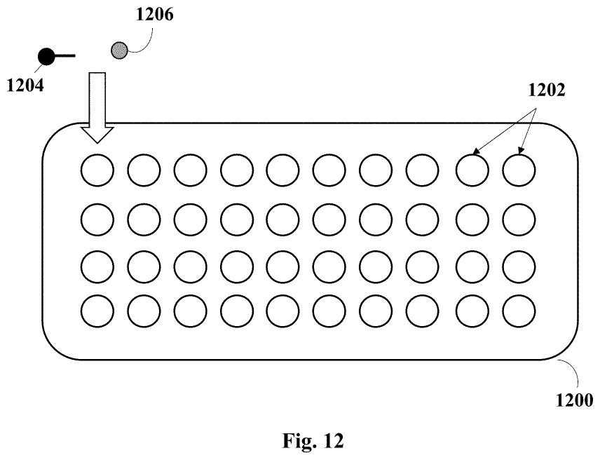

FIG. 12 schematically illustrates an example of a microwell array. The array can be contained within a substrate 1200. The substrate 1200 comprises a plurality of wells 1202. The wells 1202 may be of any size or shape, and the spacing between the wells, the number of wells per substrate, as well as the density of the wells on the substrate 1200 can be modified, depending on the application. In one such example application, a sample molecule 1206, which may comprise a cell or nucleus (e.g., a fixed cell or nucleus or an un-fixed cell/nucleus) or cellular components (e.g., nucleic acid molecules) is co-partitioned with a bead 1204, which may comprise a nucleic acid barcode molecule coupled thereto. The wells 1202 may be loaded using gravity or other loading technique (e.g., centrifugation, liquid handler, acoustic loading, optoelectronic, etc.). In some instances, at least one of the wells 1202 contains a single sample molecule 1206 (e.g., cell or nucleus) and a single bead 1204.

Reagents may be loaded into a well either sequentially or concurrently. In some cases, reagents are introduced to the device either before or after a particular operation. In some cases, reagents (which may be provided, in certain instances, in droplets or beads) are introduced sequentially such that different reactions or operations occur at different steps. The reagents (or droplets or beads) may also be loaded at operations interspersed with a reaction or operation step. For example, droplets or beads comprising reagents for fragmenting polynucleotides (e.g., restriction enzymes) and/or other enzymes (e.g., transposases, ligases, polymerases, etc.) may be loaded into the well or plurality of wells, followed by loading of droplets or beads comprising reagents for attaching nucleic acid barcode molecules to a sample nucleic acid molecule. Reagents may be provided concurrently or sequentially with a sample, such as a cell or nucleus (e.g., a fixed cell/nucleus or an un-fixed cell/nucleus) or cellular components (e.g., organelles, proteins, nucleic acid molecules, carbohydrates, lipids, etc.). Accordingly, use of wells may be useful in performing multi-step operations or reactions.

As described elsewhere herein, the nucleic acid barcode molecules and other reagents may be contained within a bead or droplet. These beads or droplets may be loaded into a partition (e.g., a microwell) before, after, or concurrently with the loading of a cell or nucleus (e.g., a fixed cell/nucleus or an un-fixed cell/nucleus), such that each cell or nucleus is contacted with a different bead or droplet. This technique may be used to attach a unique nucleic acid barcode molecule to nucleic acid molecules obtained from each cell or nucleus (e.g., a fixed cell/nucleus or an un-fixed cell/nucleus). Alternatively, or in addition to, the sample nucleic acid molecules may be attached to a support. For instance, the partition (e.g., microwell) may comprise a bead which has coupled thereto a plurality of nucleic acid barcode molecules. The sample nucleic acid molecules, or derivatives thereof, may couple or attach to the nucleic acid barcode molecules on the support. The resulting barcoded nucleic acid molecules may then be removed from the partition, and in some instances, pooled and sequenced. In such cases, the nucleic acid barcode sequences may be used to trace the origin of the sample nucleic acid molecule. For example, polynucleotides with identical barcodes may be determined to originate from the same cell/nucleus or partition, while polynucleotides with different barcodes may be determined to originate from different cells/nuclei or partitions.

The samples or reagents may be loaded in the wells or microwells using a variety of approaches. The samples (e.g., a cell, a nucleus or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In one particular non-limiting example, the microwell array or plate comprises pairs of microwells, in which each pair of microwells is configured to hold a droplet (e.g., comprising a single cell or nucleus, e.g., a single fixed cell/nucleus or a single un-fixed cell/nucleus) and a single bead (such as those described herein, which may, in some instances, also be provided or encapsulated in a droplet). The droplet and the bead (or droplet containing the bead) may be loaded simultaneously or sequentially, and the droplet and the bead may be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets comprising different reagents and/or samples, which are merged upon contact or upon application of a stimulus. In such instances, the droplet of one microwell of the pair can comprise reagents that may react with an agent in the droplet of the other microwell of the pair. For instance, one droplet can comprise reagents that are configured to release the nucleic acid barcode molecules of a bead contained in another droplet, located in the adjacent microwell. Upon merging of the droplets, the nucleic acid barcode molecules may be released from the bead into the partition (e.g., the microwell or microwell pair that are in contact), and further processing may be performed (e.g., barcoding, nucleic acid reactions, etc.). In cases where cells or nuclei, e.g., fixed cells/nuclei or un-fixed cells/nuclei are loaded in the microwells, one of the droplets may comprise reagents for further processing, e.g., lysis reagents for lysing the cell or nucleus, upon droplet merging.

A droplet may be partitioned into a well. The droplets may be selected or subjected to pre-processing prior to loading into a well. For instance, the droplets may comprise cells or nuclei, e.g., fixed cells/nuclei or un-fixed cells/nuclei, and only certain droplets, such as those containing a single cell or nucleus (or at least one cell/nucleus), may be selected for use in loading of the wells. Such a pre-selection process may be useful in efficient loading of single cells or nuclei, such as to obtain a non-Poissonian distribution, or to pre-filter cells or nuclei for a selected characteristic prior to further partitioning in the wells. Additionally, the technique may be useful in obtaining or preventing cell/nucleus doublet or multiplet formation prior to or during loading of the microwell.

In some instances, the wells can comprise nucleic acid barcode molecules attached thereto. The nucleic acid barcode molecules may be attached to a surface of the well (e.g., a wall of the well). The nucleic acid barcode molecule (e.g., a partition barcode sequence) of one well may differ from the nucleic acid barcode molecule of another well, which can permit identification of the contents contained with a single partition or well. In some cases, the nucleic acid barcode molecule can comprise a spatial barcode sequence that can identify a spatial coordinate of a well, such as within the well array or well plate. In some cases, the nucleic acid barcode molecule can comprise a unique molecular identifier for individual molecule identification. In some instances, the nucleic acid barcode molecules may be configured to attach to or capture a nucleic acid molecule within a sample or cell or nucleus (e.g., a fixed cell/nucleus or an un-fixed cell/nucleus) distributed in the well. For example, the nucleic acid barcode molecules may comprise a capture sequence that may be used to capture or hybridize to a nucleic acid molecule (e.g., RNA, DNA) within the sample. In some instances, the nucleic acid barcode molecules may be releasable from the microwell. For instance, the nucleic acid barcode molecules may comprise a chemical cross-linker which may be cleaved upon application of a stimulus (e.g., photo-, magnetic, chemical, biological, stimulus). The released nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In such cases, the unique partition barcode sequences may be used to identify the cell/nucleus or partition from which a nucleic acid molecule originated.

Characterization of samples within a well may be performed. Such characterization can include, in non-limiting examples, imaging of the sample (e.g., cell/nucleus or cellular components) or derivatives thereof. Characterization techniques such as microscopy or imaging may be useful in measuring sample profiles in fixed spatial locations. For instance, when cells or nuclei (e.g., fixed cells/nuclei or un-fixed cells/nuclei) are partitioned, optionally with beads, imaging of each microwell and the contents contained therein may provide useful information on cell/nucleus doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell/nucleus or bead loading rate, number of cell-bead pairs, cell-cell interactions (when two or more cells/nuclei are co-partitioned). Alternatively, or in addition to, imaging may be used to characterize a quantity of amplification products in the well.

In operation, a well may be loaded with a sample and reagents, simultaneously or sequentially. When cells or nuclei (e.g., fixed cells/nuclei or un-fixed cells/nuclei) are loaded, the well may be subjected to washing, e.g., to remove excess cells from the well, microwell array, or plate. Similarly, washing may be performed to remove excess beads or other reagents from the well, microwell array, or plate. In addition, the cells or nuclei may be lysed in the individual partitions to release the intracellular components or cellular analytes. Alternatively, the cells or nuclei may be fixed or permeabilized in the individual partitions. The intracellular components or cellular analytes may couple to a support, e.g., on a surface of the microwell, on a solid support (e.g., bead), or they may be collected for further downstream processing. For instance, after cell lysis, the intracellular components or cellular analytes may be transferred to individual droplets or other partitions for barcoding. Alternatively, or in addition to, the intracellular components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) may be sealed (e.g., using an oil, membrane, wax, etc.), which enables storage of the assay or selective introduction of additional reagents.

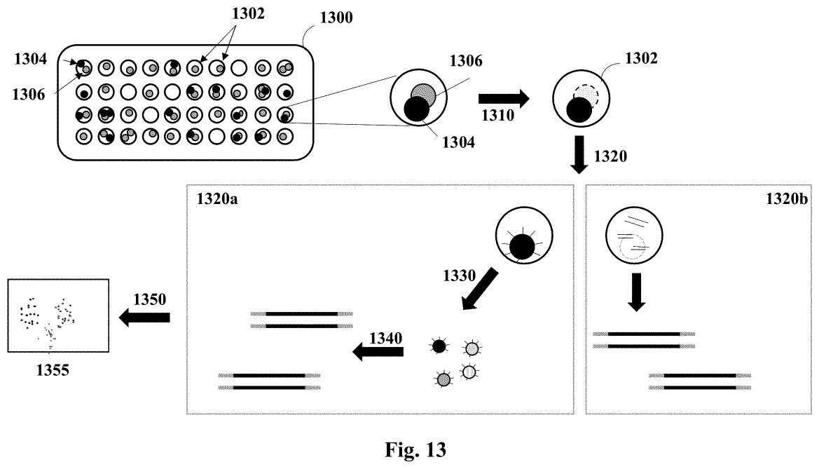

FIG. 13 schematically shows an example workflow for processing nucleic acid molecules within a sample. A substrate 1300 comprising a plurality of microwells 1302 may be provided. A sample 1306 which may comprise a cell or nucleus (e.g., a fixed cell/nucleus or an un-fixed cell/nucleus), cellular components or analytes (e.g., proteins and/or nucleic acid molecules) can be co-partitioned, in a plurality of microwells 1302, with a plurality of beads 1304 comprising nucleic acid barcode molecules. During process 1310, the sample 1306 may be processed within the partition. For instance, the cell or nucleus may be subjected to conditions sufficient to lyse the cells or nuclei (e.g., fixed cells/nuclei or un-fixed cells/nuclei) and release the analytes contained therein. In process 1320, the bead 1304 may be further processed. By way of example, processes 1320a and 1320b schematically illustrate different workflows, depending on the properties of the bead 1304.

In 1320a, the bead comprises nucleic acid barcode molecules that are attached thereto, and sample nucleic acid molecules (e.g., RNA, DNA) may attach, e.g., via hybridization of ligation, to the nucleic acid barcode molecules. Such attachment may occur on the bead. In process 1330, the beads 1304 from multiple wells 1302 may be collected and pooled. Further processing may be performed in process 1340. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1350, further characterization, such as sequencing may be performed to generate sequencing reads. The sequencing reads may yield information on individual cells or populations of cells or nuclei (e.g., fixed cells/nuclei or un-fixed cells/nuclei), which may be represented visually or graphically, e.g., in a plot 1355.

In 1320b, the bead comprises nucleic acid barcode molecules that are releasably attached thereto, as described below. The bead may degrade or otherwise release the nucleic acid barcode molecules into the well 1302; the nucleic acid barcode molecules may then be used to barcode nucleic acid molecules within the well 1302. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1350, further characterization, such as sequencing may be performed to generate sequencing reads. The sequencing reads may yield information on individual cells or populations of cells or nuclei (e.g., fixed cells/nuclei or un-fixed cells/nuclei), which may be represented visually or graphically, e.g., in a plot 1355.

### Computer Systems

The present disclosure provides computer systems that are programmed to implement methods of the disclosure. FIG. 14 shows a computer system 1401 that is programmed or otherwise configured to control a microfluidics system (e.g., fluid flow), generate partitions, or perform sequencing applications. The computer system 1401 can regulate various aspects of the present disclosure, such as, for example, regulating fluid flow rate in one or more channels in a microfluidic structure, or regulating polymerization application units. The computer system 1401 can be an electronic device of a user or a computer system that is remotely located with respect to the electronic device. The electronic device can be a mobile electronic device.

The computer system 1401 includes a central processing unit (CPU, also “processor” and “computer processor” herein) 1405, which can be a single core or multi core processor, or a plurality of processors for parallel processing. The computer system 1401 also includes memory or memory location 1410 (e.g., random-access memory, read-only memory, flash memory), electronic storage unit 1415 (e.g., hard disk), communication interface 1420 (e.g., network adapter) for communicating with one or more other systems, and peripheral devices 1425, such as cache, other memory, data storage and/or electronic display adapters. The memory 1410, storage unit 1415, interface 1420 and peripheral devices 1425 are in communication with the CPU 1405 through a communication bus (solid lines), such as a motherboard. The storage unit 1415 can be a data storage unit (or data repository) for storing data. The computer system 1401 can be operatively coupled to a computer network (“network”) 1430 with the aid of the communication interface 1420. The network 1430 can be the Internet, an internet and/or extranet, or an intranet and/or extranet that is in communication with the Internet. The network 1430 in some cases is a telecommunication and/or data network. The network 1430 can include one or more computer servers, which can enable distributed computing, such as cloud computing. The network 1430, in some cases with the aid of the computer system 1401, can implement a peer-to-peer network, which may enable devices coupled to the computer system 1401 to behave as a client or a server.

The CPU 1405 can execute a sequence of machine-readable instructions, which can be embodied in a program or software. The instructions may be stored in a memory location, such as the memory 1410. The instructions can be directed to the CPU 1405, which can subsequently program or otherwise configure the CPU 1405 to implement methods of the present disclosure. Examples of operations performed by the CPU 1405 can include fetch, decode, execute, and writeback.

The CPU 1405 can be part of a circuit, such as an integrated circuit. One or more other components of the system 1401 can be included in the circuit. In some cases, the circuit is an application specific integrated circuit (ASIC).

The storage unit 1415 can store files, such as drivers, libraries and saved programs. The storage unit 1415 can store user data, e.g., user preferences and user programs. The computer system 1401 in some cases can include one or more additional data storage units that are external to the computer system 1401, such as located on a remote server that is in communication with the computer system 1401 through an intranet or the Internet.

The computer system 1401 can communicate with one or more remote computer systems through the network 1430. For instance, the computer system 1401 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1401 via the network 1430.

Methods as described herein can be implemented by way of machine (e.g., computer processor) executable code stored on an electronic storage location of the computer system 1401, such as, for example, on the memory 1410 or electronic storage unit 1415. The machine executable or machine-readable code can be provided in the form of software. During use, the code can be executed by the processor 1405. In some cases, the code can be retrieved from the storage unit 1415 and stored on the memory 1410 for ready access by the processor 1405. In some situations, the electronic storage unit 1415 can be precluded, and machine-executable instructions are stored on memory 1410.

The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 1401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1401 can include or be in communication with an electronic display 1435 that comprises a user interface (UI) 1440 for providing, for example, the results of sequencing analysis. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1405. The algorithm can, for example, perform sequencing, analyze sequence reads, or determine sequence reads as belonging to a particular biological particle.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) from a single cell or nucleus. For example, a biological particle (e.g., a cell, nucleus or cell bead) is partitioned in a partition (e.g., droplet), and multiple analytes from the biological particle are processed for subsequent processing. The multiple analytes may be from the single cell or nucleus. This may enable, for example, simultaneous proteomic, transcriptomic, and genomic analysis of the cell or nucleus.

### Embodiments

Embodiments of the invention, which are not meant to be limiting, are described in the numbered paragraphs below.

1. A method for processing a nucleic acid molecule, comprising:

a) providing a partition comprising: (i) a fixed biological particle or a fixed membrane bound particle comprising a nucleic acid molecule comprising a nucleic acid sequence and (ii) a nucleic acid barcode molecule;

b) subjecting said partition to a condition sufficient to generate a nucleic acid molecule comprising said nucleic acid sequence coupled to said nucleic acid barcode molecule;

c) releasing said nucleic acid molecule comprising said nucleic acid sequence coupled to said nucleic acid barcode molecule from said partition to generate a released nucleic acid molecule comprising said nucleic acid sequence coupled to said nucleic acid barcode molecule; and

d) subjecting said released nucleic acid molecule comprising said nucleic acid sequence coupled to said nucleic acid barcode molecule to a condition sufficient to extend said nucleic acid barcode molecule (e.g., a 3′ end of said nucleic acid barcode molecule), using said nucleic acid sequence as a template, to generate a barcoded nucleic acid molecule.

2. The method of paragraph 1, wherein said nucleic acid molecule is a cross-linked nucleic acid molecule.

3. The method of paragraph 2, wherein b) comprises (i) generating an unlinked nucleic acid molecule from said cross-linked nucleic acid molecule and (ii) allowing said unlinked nucleic acid molecule to couple with said nucleic acid barcode molecule.

4. The method of paragraph 1, wherein said partition further comprises a cleaving agent.

5. The method of paragraph 4, wherein said cleaving agent is a protease.

6. The method of paragraph 5, wherein said protease is selected from the group consisting of Proteinase K and Subtilisin A.

7. The method of paragraph 4, wherein said cleaving agent is a catalyst.

8. The method of paragraph 4, wherein said cleaving agent breaks a bond generated by a fixation agent.

9. The method of paragraph 1, wherein b) comprises heating said partition.

10. The method of paragraph 9, wherein b) comprises subjecting said partition to temperature of 53° C. for 45 minutes and then a temperature of 70° C. for 15 minutes.

11. The method of paragraph 10, wherein b) further comprises subjecting said partition to a temperature of 90° C. for 10 minutes.

12. The method of paragraph 1, further comprising, subsequent to b) and prior to c), cooling said partition or allowing said partition to cool.

13. The method of paragraph 1, wherein d) is performed in a presence of a protease inhibitor.

14. The method of paragraph 1, wherein d) comprises using a reverse transcriptase to extend said nucleic acid barcode molecule (e.g., a 3′ end of said nucleic acid molecule barcode molecule).

15. The method of paragraph 14, wherein said reverse transcriptase comprises RNase activity.

16. The method of paragraph 1, wherein said nucleic acid barcode molecule comprises a capture sequence configured to anneal to a nucleic acid molecule of said fixed biological particle or said fixed membrane bound particle.

17. The method of paragraph 16, wherein said capture sequence is a poly-T sequence.

18. The method of paragraph 1, wherein said nucleic acid barcode molecule comprises a unique molecular identifier sequence (UMI), or a sequence configured to allow attachment to a flow cell.

19. The method of paragraph 1, further comprising appending an additional sequence to said barcoded nucleic acid molecule.

20. The method of paragraph 19, wherein said additional sequence is a poly-C sequence.

21. The method of paragraph 19, wherein said appending is performed by a ligase or a polymerase.

22. The method of paragraph 21, wherein said polymerase is a reverse transcriptase.

23. The method of paragraph 19, wherein said appending comprises using a splint nucleic acid molecule.

24. The method of paragraph 19, wherein said appending comprises using a primer to anneal to said barcoded nucleic acid molecule.

25. The method of paragraph 19, wherein said partition further comprises a template switching oligonucleotide (TSO).

26. The method of paragraph 25, further comprising subjecting said partition to a condition sufficient to hybridize said TSO to said additional sequence.

27. The method of paragraph 25, further comprising extending said barcoded nucleic acid molecule to generate an extended barcoded nucleic acid molecule comprising a sequence complementary to said TSO.

28. The method of paragraph 27, wherein said TSO comprises a sequencing primer sequence or complement thereof.

29. The method of paragraph 1, wherein said nucleic acid barcode molecule is coupled to a support.

30. The method of paragraph 29, wherein said support is a bead.

31. The method of paragraph 30, wherein said bead is a gel bead.

32. The method of paragraph 29, wherein said nucleic acid barcode molecule is coupled to said support by a labile moiety.

33. The method of paragraph 1, wherein said partition is a droplet or a well.

34. The method of paragraph 33, wherein said releasing comprises breaking said droplet.

35. The method of paragraph 2, wherein said cross-linked nucleic acid molecules comprises a ribonucleic acid (RNA) molecule.

36. The method of paragraph 35, wherein said RNA molecule is a messenger RNA (mRNA) molecule.

37. The method of paragraph 1, further comprising (e) sequencing said barcoded nucleic acid molecule or an amplification product thereof.

38. The method of paragraph 1, further comprising providing a plurality of partitions.

39. The method of paragraph 38, further comprising, prior to a), partitioning a plurality of fixed biological particles or a plurality of fixed membrane bound particles into a plurality of partitions.

40. The method of paragraph 38, wherein subsequent to b) and prior to c), said plurality of partitions comprise a plurality of nucleic acid molecules coupled to nucleic acid barcode molecules.

41. The method of paragraph 40, further comprising subsequent to c), pooling said plurality of nucleic acid molecules coupled to said nucleic acid barcode molecules from said plurality of partitions.

42. The method of paragraph 1, further comprising prior to a), fixing a biological particle or a membrane bound particle to generate said fixed biological particle or said fixed membrane bound particle.

43. The method of paragraph 42, wherein said fixing comprises use of a fixation agent.

44. The method of paragraph 43, wherein said fixation agent comprises paraformaldehyde.

45. The method of paragraph 1, wherein said fixed biological particle or said fixed membrane bound particle comprises a cell, nucleus, virus, or nucleus.

46. A composition, comprising a reverse transcriptase (RT), a protease and a polyethylene glycol (PEG).

47. The composition of paragraph 46, comprising a nucleic acid template capable of being used by the RT to synthesize a complementary DNA strand.

48. The composition of paragraph 47, wherein the nucleic acid template includes ribonucleic acid (RNA).

49. The composition of paragraph 48, wherein the nucleic acid template is from a cell or a nucleus.

50. The composition of paragraph 49, wherein the cell or nucleus is fixed and at least partially un-fixed.

51. The composition of paragraph 50, wherein the protease is capable of un-fixing or contributing to un-fixing of biomolecules in the fixed cell or nucleus.

52. The composition of any one of paragraphs 47-51, comprising a first primer capable of being used by the RT to initiate first-stand synthesis of the complementary DNA strand.

53. The composition of any one of paragraphs 47-52, including deoxyribonucleotides (dNTPs) and magnesium ions (Mg2+) or manganese ions (Mn2⁺).

54. The composition of paragraph 53, comprising a TS oligo (TSO).

55. The composition of paragraph 54, wherein the RT is capable of using the TSO as template to synthesize a DNA strand that is longer than a complementary DNA strand synthesized without the TSO.

56. The composition of any one of paragraphs 46-55, wherein the RT is or is derived from Avian myeloblastosis virus (AMV) RT or Moloney Murine leukemia virus (MMLV) RT.

57. The composition of any one of paragraphs 46-55, wherein the RT includes variants of Avian myeloblastosis virus (AMV) RT or Moloney Murine leukemia virus (MMLV) RT.

58. The composition of any one of paragraphs 46-57, wherein the RT has template switching (TS) activity.

59. The composition of any one of paragraphs 46-58, wherein the RT has terminal transferase activity.

60. The composition of any one of paragraphs 46-59, wherein the PEG comprises oligomers or polymers that have an average molecular mass between about 1,000-16,000 g/mol; 2,000-14,000 g/mol; 3,000-12,000 g/mol; 4,000-10,000 g/mol; or 5,000-9,000 g/mol.

61. The composition of any one of paragraphs 46-60, wherein a final concentration of PEG in the composition is between about 1-12%, 2-11%, 3-10% or 4-8%.

62. The composition of one of paragraphs 53 or 54, wherein an amount of DNA synthesized by the RT in the composition is greater than an amount of DNA synthesized by the RT in an equivalent composition not comprising PEG.

63. The composition of one of paragraphs 53 or 54, wherein the composition comprises a second primer capable of being used by the RT to initiate second-strand DNA synthesis.

64. The composition of any one of paragraphs 46-63, wherein the PEG is attached to the RT.

65. A method, comprising synthesizing a complementary single-stranded DNA in a composition that includes a reverse transcriptase (RT), a nucleic acid template, a protease, and a polyethylene glycol (PEG).

66. The method of paragraph 65, wherein the composition additionally includes a first primer for initiation of first-strand synthesis of the complementary single-stranded DNA.

67. The method of paragraph 66, wherein the composition includes deoxyribonucleotides (dNTPs), and magnesium ions (Mg²⁺) or manganese ions (Mn²⁺).

68. The method of paragraph 67, wherein the RT has template switching (TS) activity.

69. The method of paragraph 68, wherein the composition additionally includes a TS oligo (TSO).

70. The method of paragraph 69, wherein at least some of the synthesized complementary single-stranded DNA is longer than a complementary single-stranded DNA synthesized in absence of the TSO.

71. The method of any one of paragraphs 67-70, wherein the composition comprises a second primer capable of being used by the RT to initiate second-strand DNA synthesis.

72. The method of any one of paragraphs 67-71, where the complementary single stranded DNA synthesis and/or the second-strand DNA synthesis is performed in a discrete droplet, where the droplet optionally includes a single cell, nucleus and/or biomolecules from a single cell or nucleus.

73. The method of any one of paragraphs 65-72, wherein the nucleic acid template includes ribonucleic acid (RNA).

74. The method of paragraph 73, wherein the RNA is from or part of fixed cells or nuclei that have been un-fixed or partially un-fixed.

75. The method of paragraph 74, wherein the protease is capable of un-fixing or contributing to un-fixing of the fixed cells or nuclei.

76. The method of any one of paragraphs 65-75, wherein the PEG comprises oligomers or polymers that have an average molecular mass between about 1,000-16,000 g/mol; 2,000-14,000 g/mol; 3,000-12,000 g/mol; 4,000-10,000 g/mol; or 5,000-9,000 g/mol.

77. The method of any one of paragraphs 65-76, wherein a final concentration of PEG in the composition is between about 1-12%, 2-11%, 3-10% or 4-8%.

78. The method of any one of paragraphs 65-77, wherein the PEG is attached to the RT.

79. A method, comprising:

(a) un-fixing or partially un-fixing fixed cells, nuclei or tissues using an un-fixing agent; and

(b) synthesizing a complementary single-stranded DNA using a nucleic acid template from the un-fixed/partially un-fixed cells, nuclei or tissues and a reverse transcriptase (RT), in presence of a polyethylene glycol (PEG).

80. The method of paragraph 79, wherein the un-fixing agent is capable of removing crosslinks formed in biomolecules in the cells or nuclei by fixation with an aldehyde (e.g., paraformaldehyde, glutaraldehyde), an NHS ester (e.g., N-Hydroxysuccinimide), an imidoesters, or a combination thereof.

81. The method of paragraph 79, wherein the un-fixing agent is capable of removing crosslinks formed in biomolecules by fixation with paraformaldehyde; optionally, fixation with a PF solution at a concentration of 1%-4% PFA.

82. The method of any one of paragraphs 79-81, wherein the un-fixing agent has protease activity.

83. The method of any one of paragraphs 79-82, wherein the un-fixing agent includes a protease selected from the group consisting of Proteinase K and a subtilisin.

84. The method of any one of paragraphs 79-83, wherein the PEG comprises oligomers or polymers that have an average molecular mass between about 1,000-16,000 g/mol; 2,000-14,000 g/mol; 3,000-12,000 g/mol; 4,000-10,000 g/mol; or 5,000-9,000 g/mol.

85. The method of any one of paragraphs 79-84, wherein a final concentration of PEG in step (b) is between about 1-12%, 2-11%, 3-10% or 4-8%.

86. The method of any one of paragraphs 79-85, wherein step (a) is performed in presence of a polyethylene glycol (PEG).

87. The method of paragraph 86, wherein the PEG in step (a) comprises oligomers or polymers that have an average molecular mass between about 1,000-16,000 g/mol; 2,000-14,000 g/mol; 3,000-12,000 g/mol; 4,000-10,000 g/mol; or 5,000-9,000 g/mol.

88. The method of one of paragraphs 86 or 87, wherein a final concentration of PEG in step (a) is between about 1-12%, 2-11%, 3-10% or 4-8%.

89. The method of any one of paragraphs 79-88, wherein step (b) is performed in presence of protease activity.

90. The method of any one of paragraphs 79-89, comprising, prior to step (a), fixing cells or nuclei with a fixing agent.

91. The method of paragraph 90, wherein the fixing agent is selected from the group consisting of an aldehyde (e.g., paraformaldehyde, glutaraldehyde), an NHS ester (e.g., N-Hydroxysuccinimide), an imidoesters, or a combination thereof.

92. The method of paragraph 90, wherein the fixing reagent includes paraformaldehyde (“PFA”); optionally, the fixing reagent is a PFA solution at a concentration of 1%-4% PFA.

93. The method of any one of paragraphs 79-92, comprising, subsequent to step (b), synthesizing a second-strand DNA using the complementary single-stranded DNA as template.

94. The method of any one of paragraphs 79-93, wherein in step (b) the PEG is attached to the RT.

95. A kit, comprising a reverse transcriptase (RT) and a polyethylene glycol (PEG).

96. The kit of paragraph 95, additionally including an un-fixing agent, which includes a protease.

97. The kit of paragraph 96, additionally including a fixing agent.

98. The kit of any one of paragraphs 95-97, wherein the PEG is attached to the RT.

99. A method, comprising un-fixing cells, nuclei or tissues that have been fixed with a fixing agent, wherein the un-fixing is performed using an un-fixing agent in presence of polyethylene glycol (PEG).

100. The method of paragraph 99, wherein the fixing agent is selected from the group consisting of an aldehyde (e.g., paraformaldehyde, glutaraldehyde), an NHS ester (e.g., N-Hydroxysuccinimide), an imidoesters, or a combination thereof.

101. The method of paragraph 99, wherein the fixing reagent includes paraformaldehyde (“PFA”); optionally, the fixing reagent is a PFA solution at a concentration of 1%-4% PFA.

102. The method of any one of paragraphs 99-101, wherein the un-fixing agent is capable of removing crosslinks formed in biomolecules in the cells or nuclei by fixation with an aldehyde (e.g., paraformaldehyde, glutaraldehyde), an NHS ester (e.g., N-Hydroxysuccinimide), an imidoesters, or a combination thereof.

103. The method of any one of paragraphs 99-101, wherein the un-fixing agent is capable of removing crosslinks formed in biomolecules by fixation with paraformaldehyde; optionally, fixation with a PF solution at a concentration of 1%-4% PFA.

104. The method of any one of paragraphs 99-103, wherein the un-fixing agent has protease activity.

105. The method of any one of paragraphs 99-104, wherein the un-fixing agent includes Proteinase K and/or a subtilisin.

106. The method of any one of paragraphs 99-105, comprising an additional step of using biomolecules from the un-fixed cells or nuclei as a substrate or template in an enzymatic reaction.

107. The method of paragraph 106, wherein a nucleic acid from the un-fixed cells or nuclei is used as a template in a DNA synthesis reaction.

108. The method of paragraph 107, wherein the nucleic acid includes ribonucleic acid (RNA) or deoxyribonucleic acid (DNA).

109. The method of any one of paragraphs 107-108, wherein an enzyme used in the enzymatic reaction includes a DNA polymerase, reverse transcriptase (RT), or RNA polymerase.

110. The method of any one of paragraphs 99-105, comprising an additional step of isolating a nucleic acid from the un-fixed cells or nuclei.

111. A method, comprising:

(a) fixing cells or nuclei with a fixing agent to crosslink biomolecules in the cells or nuclei; and

(b) un-fixing the fixed cells or nuclei with an un-fixing agent in the presence of polyethylene glycol (PEG) to remove at least some crosslinks in the biomolecules.

112. The method of paragraph 111, wherein the fixing agent is selected from the group consisting of an aldehyde (e.g., paraformaldehyde, glutaraldehyde), an NHS ester (e.g., N-Hydroxysuccinimide), an imidoesters, or a combination thereof.

113. The method of paragraph 111 wherein the fixing reagent includes paraformaldehyde (“PFA”); optionally, the fixing reagent is a PFA solution at a concentration of 1%-4% PFA.

114. The method of any one of paragraphs 111-113, wherein the un-fixing agent is capable of removing crosslinks formed in biomolecules in the cells or nuclei by fixation with an aldehyde (e.g., paraformaldehyde, glutaraldehyde), an NHS ester (e.g., N-Hydroxysuccinimide), an imidoesters, or a combination thereof.

115. The method of any one of paragraphs 111-113, wherein the un-fixing agent is capable of removing crosslinks formed in biomolecules by fixation with paraformaldehyde; optionally, fixation with a PF solution at a concentration of 1%-4% PFA.

116. The method of any one of paragraphs 111-115, wherein the un-fixing agent has protease activity.

117. The method of any one of paragraphs 111-116, wherein the un-fixing agent includes Proteinase K and/or a subtilisin.

118. The method of any one of paragraphs 111-117, comprising an additional step (c) of isolating a nucleic acid from the un-fixed cells or nuclei.

119. The method of paragraph 118, wherein the nucleic acid includes ribonucleic acid (RNA).

120. The method of paragraph 119, wherein an amount of RNA isolated is greater than an amount of RNA isolated from an equivalent number of fixed cells or nuclei in which step (b) was performed without PEG.

121. A composition comprising a plurality of partitions, wherein a partition of said plurality of partitions comprises a reverse transcriptase (RT), a protease and a polyethylene glycol (PEG).

122. The composition of paragraph 121, wherein said plurality of partitions is a plurality of droplets or a plurality of wells.

123. The composition of paragraph 121, wherein said partition further comprises a nucleic acid template molecule.

124. The composition of paragraph 123, wherein said nucleic acid template molecule comprises RNA.

125. The composition of paragraph 123, wherein said nucleic acid template is derived from a cell or nucleus.

126. The composition of paragraph 125, wherein said cell is a fixed cell or said nucleus is a fixed nucleus.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

### EXAMPLES

The following examples are for illustrating various embodiments and are not to be construed as limitations.

### Example 1: Barcoding Nucleic Acid Molecules Derived from Fixed Membrane Bound Particles

Membrane bound particles, here either fresh (unfixed) or paraformaldehyde fixed (4%) Jurkat cells were used.

The fresh Jurkat cells were used as a positive control. The fresh Jurkat cells (sometimes referred to as sample “SOP” in these Examples) were processed as single cells in droplets using 10× Genomics 3′ RNA-seq Gene Expression (3′ v3 GEX).

The fixed membrane bound particles were resuspended in a master mix with cleaving agents. In various examples, a protease (e.g., Proteinase K, or CAP, i.e., subtilisin A) was used, a catalyst was used, or a protease and a catalyst were used. The fixed membrane bound particles, cleavage agents and nucleic acid barcode molecules, attached to a support (a bead), were co-partitioned into partitions. The partitions were then heated, and the cleaving agents were activated. The partition was heated to 53° C. for 45 mins followed by a heating to 70° C. for 15 mins.

The nucleic acid molecules which had been subjected to the cleavage agents were de-crosslinked such that the nucleic acid molecules could anneal with the nucleic acid barcode molecules. In particular, the nucleic acid barcode molecules comprised poly-T tails and could specifically capture the mRNA in the cells. The nucleic acid barcode molecules comprised a Read 1 sequence, a barcode sequence, a UMI sequences, and a capture sequence (poly-T) for capturing nucleic acid molecules for the fixed membrane bound particles. The barcode sequence in each partition was different and was used to identify nucleic acid molecule as belonging to a particular partition. Barcoded nucleic acid molecules generated from the methods comprised a barcode indicative of a single cell and the cDNA sequence derived from an mRNA. Upon pooling and sequencing in bulk, cDNA sequences belonging to a particular could be grouped and the transcription profile single fixed cells was elucidated.

To perform the above, the partitions were cooled to 25° C. such that the annealed nucleic acid molecules remained annealed. The nucleic acid molecules were then released from the partitions by breaking of the partitions.

Upon release of the nucleic acid molecule from the partition, the nucleic acid molecules were extended to generate a barcoded nucleic acid molecule.

Here, an extension reaction was performed on the nucleic acid barcode molecule in the bulk solution. The extension reaction was performed with a template switching reverse transcriptase in the presence of a template switching oligo (TSO). This reaction was performed at 53° C. for 45 mins followed by heating to 85° C. for 15 mins.

In another potential example processing (not used in this Example), the released nucleic acid molecules from the partition are subjected to reverse transcription reaction using reverse transcriptase comprising RNase activity and without template switching. As opposed to using a TSO in conjunction with a template switching reverse transcription, a ligation reaction may be used. An App ligase and a 5′ App TSO oligonucleotide may be used to ligate the TSO sequence with specificity determined by the App ligase specificity to particular substrates. This reaction may be run at 65° C. for 18 hours. Alternatively, a splint ligation using a ligase (such as a T4 ligase), may be performed using a 5′P TSO, and a 5′ reverse complement TSO (rcTSO)-8N oligonucleotide splint. The splint may anneal to the TSO and the 8N may anneal to a sequence of a nucleic acid molecule of interest. Upon incubating at 25° C. for 1 hr., followed by a 90° C. for 10 mins, the nucleic acid molecule or interest and the TSO may be ligated together.

After subjecting the nucleic acid barcode molecule sequences to an extension reaction, a primer was used to extend the complementary strand. A 5′ rcTSO-6N primer was used and incubated at 25° C. for 45 mins, followed by 90° C. for 10 mins, for the complementary strand to be generated.

The barcoded nucleic acid molecule generated was then subjected to clean up and amplification reactions and then sequenced to generate sequence reads.

In the work performed in this Example, single-cell UMI profiles were collapsed across each entire sample to generate pseudo-bulk profiles for each gene detected. The pseudo-bulk profile for one condition (i.e., fresh cells, fixed cells unfixed using different cleaving agents) was compared with another condition by plotting one condition on the x-axis and the other condition on the y-axis. A line was drawn though the points and R² was determined to correlate the results obtained by the two compared methods. The R² values obtained from the example study are shown below:

Proteinase K protease+catalysts vs. Fresh cells, R²=0.59;

CAP protease+catalysts vs. Fresh cells, R²=0.44; and

CAP protease+catalysts vs. Proteinase K+catalysts, R²=0.966.

These data show that, using the methods disclosed herein, single-cell gene expression sequence data can be obtained from un-fixed single cells. In this example, single cells were un-fixed in partitions, and extension/amplification reactions were performed in bulk.

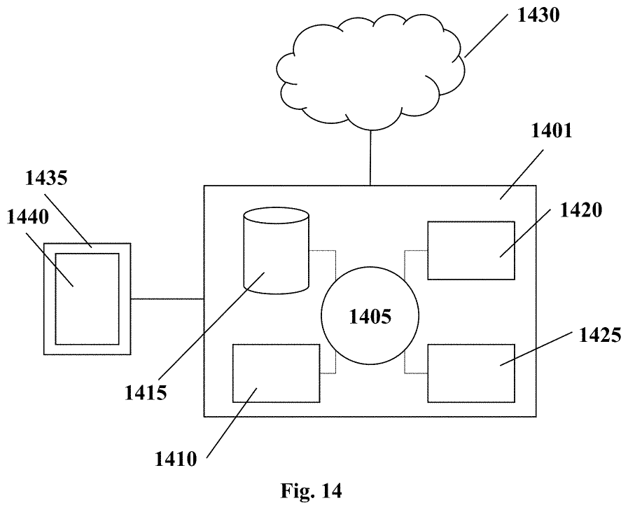

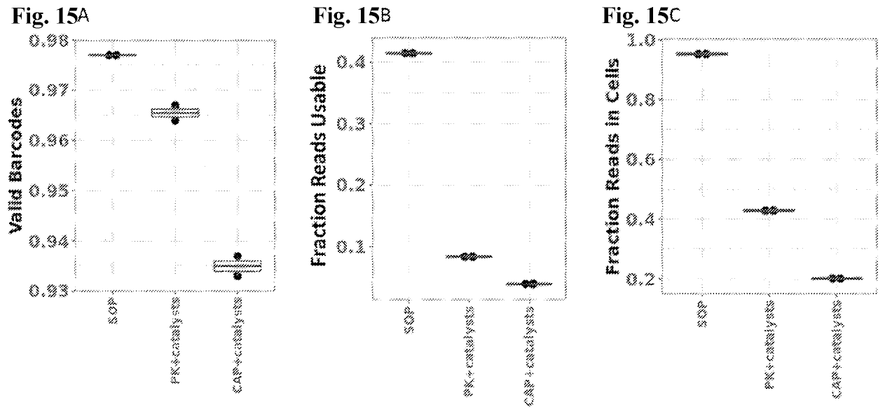

In another analysis, the single-cell libraries obtained under the various conditions described above were compared. These data are shown in FIGS. 15A, 15B and 15C.

FIG. 15A compares the relative number of valid barcodes present in libraries prepared from fresh cells (shown as “SOP”), cells un-fixed using Proteinase K protease+catalysts, and cells unfixed using cold-active protease (CAP)+catalysts. Likewise, FIG. 15B compares the fraction of usable reads present in these different libraries. FIG. 15C compares the fraction of reads in cells. Although these library quality metrics for the un-fixed cells are lower than for the library made from fresh cells, the data demonstrate that reads can be obtained from fixed and un-fixed single cells, using the conditions disclosed here.

In another analysis of the libraries, the complexity of genes and UMIs in libraries obtained from un-fixed cells was lower than the complexity of the libraries obtained from fresh cells. However, the data demonstrated that such data can be obtained from un-fixed single cells.

### Example 2: Analysis of Un-Fixed Cells Processed in GEMs, Using Various Combinations of Un-Fixing Agents

Fixed cells were decrosslinked/un-fixed and then processed using the Chromium Single Cell 3′ v3 protocols and reagents (10×Genomics, Inc.). Several different conditions for reversing PFA fixation in GEMs were tested as summarized in Table 2, below. Data for un-fixing under various conditions was correlated with data obtained from un-fixed, fresh cells, using the Pearson correlation coefficient.

TABLE 2
Un-Fixing/Decrosslinking Conditions

| | | | Pearson Correlation |
|---|---|---|---|
| | | | Coefficient Compared |
| Condition | Protease | Catalyst(s) | to Fresh Cells |
| 1 | Proteinase K | N/A | 0.473 |
| 2 | Proteinase K | Compound 1 | N/A |
| 3 | Proteinase K | Compound 8 | N/A |
| 4 | Proteinase K | Compounds 1 + 8 | 0.564 |
| 5 | Subtilisin A | N/A | 0.465 |
| 6 | Subtilisin A | Compound 1 | N/A |
| 7 | Subtilisin A | Compound 8 | N/A |
| 8 | Subtilisin A | Compounds 1 + 8 | 0.421 |

For negative controls, fresh, unfixed Jurkat cells were similarly processed to reverse fixation, as in Table 2.

The cells (fixed and un-fixed; un-fixed controls), reagents, 10× Genomics 3′ gel beads (containing nucleic acid barcode molecules) were co-partitioned into GEMs. GEMs were then heated to activate the reagents for reversing fixation. GEMs were heated to 53° C. for 45 mins followed by a heating to 70° C. for 15 mins. The GEMs were cooled to 25° C. such that the annealed nucleic acid molecules may remain annealed. The emulsions were broken, and the contents pooled for reverse transcription in bulk. The resulting libraries were analyzed.

Based on single cell UMI profiles and gene expression correlations computed as the Pearson correlation (R²) of gene expression counts between samples, feasibility for in-GEM de-crosslinking followed by bulk reverse transcription was demonstrated.

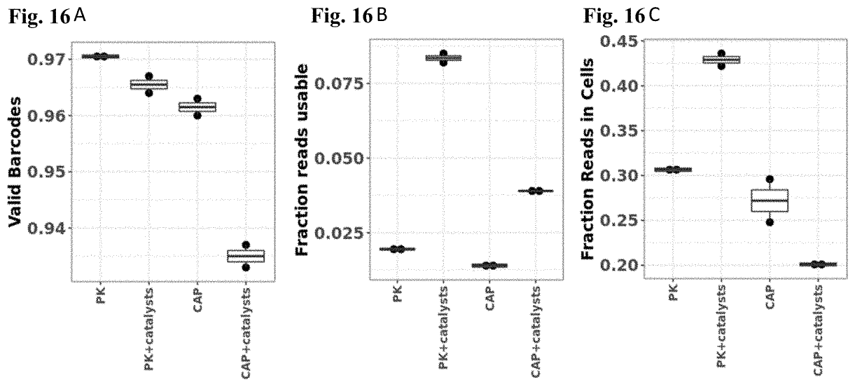

In addition, a study similar to that described in Example 1 was performed to determine properties of single-cell libraries obtained from fixed cells that were un-fixed by proteases alone, compared with libraries obtained from fixed cells that were un-fixed by proteases in combination with catalysts. These data are shown in FIGS. 16A, 16B and 16C.

FIG. 16A compares the relative number of valid barcodes present in libraries prepared from cells unfixed by Proteinase K protease (PK), PK in combination with catalysts, cold-active protease (CAP) or CAP in combination with catalysts. Likewise, FIG. 16B compares the fraction of usable reads present in these different libraries. FIG. 16C compares the fraction of reads in cells. The data show that at least some parameters of libraries obtained from cells un-fixed by proteases in combination with catalysts were improved as compared to libraries obtained from cells un-fixed by proteases alone. The data show, however, that under all conditions (proteases, proteases+catalysts), sequences were obtained from single cells which were un-fixed in partitions.

In an addition to this study, in looking at reads from a single gene, GRCh38, the number of reads mapped confidentially to the transcript in libraries obtained from cells un-fixed with either Proteinase K or cold-active protease, were significantly increased when the cells were un-fixed with either Proteinase K+catalysts or cold-active protease+catalysts, respectively.

### Example 3: Polyethylene Glycol Increases Reverse Transcriptase Activities in Presence of Proteases

Since RNA from un-fixed cells may be used as template for reverse transcriptase (RT) reactions, and since proteases are used in some cell unfixing reactions, RT activity in the presence of two different un-fixing proteases was tested. Generally, proteases were found to decrease RT activities. Different substances were tested for their ability to reverse the effects of proteases on RT activity. First-strand RT reactions were performed in the presence of a known polyadenylated RNA template, an oligo dT primer, and a template switching oligo (TSO). The reverse transcriptase was Moloney Murine Leukemia Virus (MMLV) reverse transcriptase variant 42B (U.S. Patent Application Publication No. 2018/0312822, herein incorporated by reference). The reactions were run under conditions permissive for both full-length first strand DNA synthesis, and for DNA synthesis that extends the length of the 3′ end of the first strand through use of the TSO as template to produce a DNA stand that is longer than the full-length first stand DNA synthesis product. After the reactions had run either 45 or 90 min., the reactions were stopped, and the input and reaction products were separated by capillary electrophoresis (CE) and the amounts of full-length and full-length+TSO (generally just called “TSO”) first strands produced were determined from the peaks (see the diagram after the next paragraph).

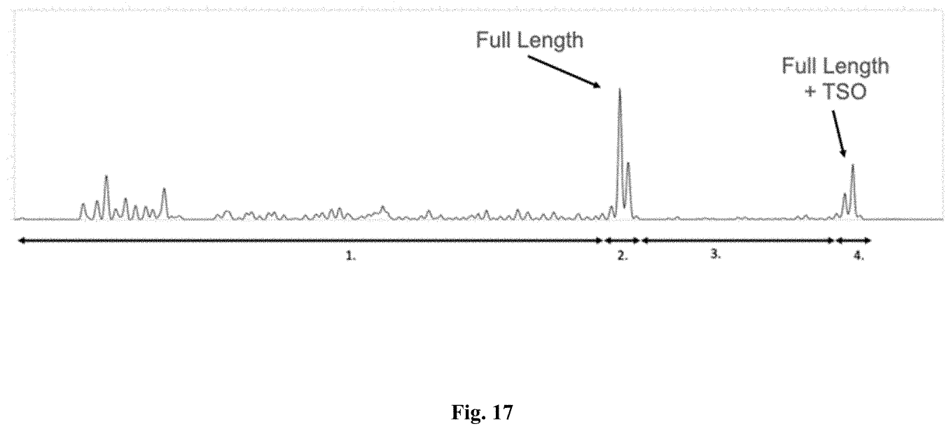

In more detail, the reactions were performed in 20 μl reaction volumes that included primer, template, TSO, RT enzyme, water, and GEM-U Reagent and were run on a thermocycler at 25° C. starting temperature, 53° C. for 45 minutes, 85° C. for 5 minutes, then held at 4° C. The reaction products were diluted 1:20 in Formamide-std mix containing 1:20 GS120Liz Size Standard, and 18:20 HiDi Formamide, then heated for 95° C. for 5 minutes and 4° C. for 2 minutes before loading onto an Applied Biosystems SegStudio™ and running the Fragment Analysis program using standard settings. An example output is shown in FIG. 17. As indicated in FIG. 17, areas under the peaks shown in the trace were designated as 1 (incomplete), 2 (elongation plus tailing), 3 (incomplete TSO) and 4 (complete TSO).

Using the areas, as indicated above, Transcription Efficiency was calculated by dividing the area of the full length and TSO peak products by total area of product. Transcription=Area (2+3+4)/Area (1+2+3+4).

TSO Efficiency was calculated by dividing TSO peak area by total product area. TSO Efficiency=Area 4/Area (1+2+3+4).

Nucleotide sequences used in the reactions were as follows: Primer:

(SEQ ID NO: 1)
/56-FAM/CGA CTC ACT GAC ACT CGC;
Template, 82 bp, GC Content 56.1%:
(SEQ ID NO 2)
rArCrG rArCrC rGrUrC rGrUrC rArUrG
rUrArG rCrGrU rUrUrG rUrCrG rGrArG
rArCrU rCrCrU rArGrA rUrCrA rGrArU
rGrUrC rCrUrC rCrUrG rGrCrU rArCrU
rGrCrA rCrGrC rGrArG rUrGrU rCrArG
rUrGrA rGrUrC rG;
TSO:
(SEQ ID NO: 3)
AAG CAG TGG TAT CAA CGC AGA GTA CAT
rGrGrG.

GEM-U Reagent contained 8% (v/v) Glycerol, 50 mM Tris pH 8.0, 4.22 mM Tris pH 7.5, 8.44 mM Sodium Chloride, 3 mM Magnesium Chloride, 75 mM Potassium Chloride, 0.5% (w/v) Synperonic F108, 0.16 mM dNTPs, 399 ug/mL BSA, 20.8 mM DTT, 0.086× Enzyme Storage Buffer, 0.008% (v/v) Triton-X=100, 0.084 mM EDTA, 0.11% (w/v) DBDM, and 0.5% (w/v) Polyacrylamide.

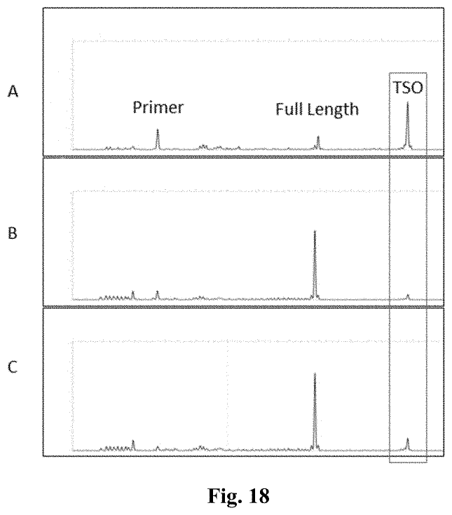

FIGS. 18A, 18B and 18C show example CE traces from various RT reactions. Separated peaks of input primers, full-length first strand RT product, and TSO products are shown. The height of each peak indicates the relative amount of each species in the peak. FIG. 18A shows results of a standard reverse transcriptase reaction, as described above. FIG. 18B shows results of an example RT reaction as above, where the reaction mixture additionally contained Proteinase K. The data shown that, compared to FIG. 18A, that addition of Proteinase K significantly inhibits RT synthesis of the product that uses the TSO to produce the extended first strand. FIG. 18C shows an example RT reaction where, in addition to Proteinase K, polyethylene glycol (PEG) 6000 was added to a final concentration of 8% in the reaction mixture. These data show, as compared to FIG. 18B, an increase in the amount of the extended-length TSO product as compared to the full-length product in FIG. 18C. These data show that Proteinase K decreases RT synthesis of TSO product and that addition of PEG partially reverses inhibition of synthesis of the TSO product.

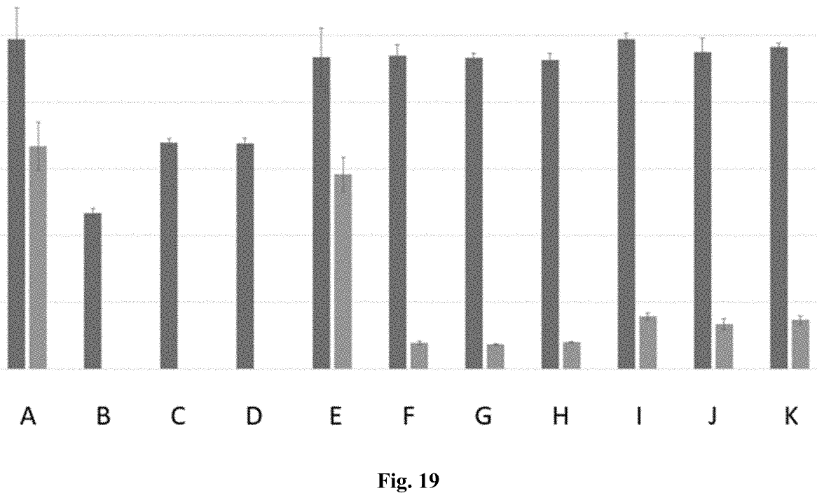

Additional studies were performed to examine the ability of PEG, and other substances, to reduce or prevent reduction of RT activities in the presence of various proteases. RT reactions were performed to test effects of an additional protease, subtilisin A, on RT and to test whether detrimental effects of subtilisin A and Proteinase K on RT could be reversed by PEG or by a broad-spectrum protease inhibitor. The reactions were performed as described above and analyzed by CE. The CE results for each reaction were plotted on a graph, as shown in FIG. 19. The relative amounts of full-length first-strand product (indicated as Transcription Efficiency in FIG. 19; left bar for each reaction sample in the graph) and extended-length TSO first-strand product (indicated as TSO Efficiency in FIG. 19; right bar for each reaction sample in the graph). All reactions were run for either 45 or 90 minutes. All reactions contained template, primers and reverse transcriptase, as described above. Other additions to the reactions, and conditions for the reactions were as follows:

(A) No additions, 45 min.;

(B) Subtilisin A, 45 min.;

(C) Subtilisin A and PEG 6000 (8% final concentration); 45 min.;

(D) Subtilisin A, PEG 6000 (8% final concentration) and protease inhibitor; 45 min.;

(E) No additions, 90 min.;

(F) Proteinase K; 45 min.;

(G) Proteinase K; 90 min.;

(H) Proteinase K and protease inhibitor; 90 min.;

(I) Proteinase K and PEG 6000 (8% final concentration); 45 min.;

(J) Proteinase K, PEG 6000 (8% final concentration) and protease inhibitor; 90 min.; and

(K) Proteinase K and PEG 6000 (4% final concentration); 45 min.

Samples A, F and I of FIG. 19 show similar data to that shown in FIGS. 18A, 18B and 18C, respectively. In the control reaction, RT produces a full-length first-strand DNA and an extended-length first strand TSO product (sample A). Addition of Proteinase K to the reaction has little effect on full-length first strand synthesis, but significantly decreases synthesis of the extended-length first strand TSO product (sample F). Addition of PEG 6000 to a final concentration of 8% in the reaction rescues synthesis of some of the TSO product (sample I). Addition of PEG 6000 to a final concentration of 4% rescues synthesis of TSO product to about the same degree as 8% PEG (sample K).

When Proteinase K is replaced with a different protease—subtilisin A—another result is obtained. Here, subtilisin A eliminated the ability of RT to produce the extended-length first strand TSO products (compare sample B with sample A), like Proteinase K (sample F). But subtilisin A also decreases the ability of RT to produce full-length first strand product (sample B). PEG addition to the reaction rescues some of the RT's ability to produce full-length product (sample C), but PEG does not detectably rescue any of the ability of RT to produce TSO product in presence of the subtilisin A enzyme. Adding a broad-spectrum protease inhibitor to the reaction containing PEG (sample D) produced the same result as PEG alone.

The above reactions were all carried out for 45 min. In reactions performed for 90 min., Proteinase K decreased RT ability to synthesize TSO product (compare sample G to sample E). Addition of the broad-spectrum protease inhibitor to a sample containing Proteinase K (sample H) did not rescue TSO activity. When PEG 6000 at an 8% final concentration is added to the reaction containing Proteinase K and the protease inhibitor (sample J), some of the ability of RT to produce TSO products was rescued.

Using the conditions described here, generally about 30% of Full-Length product is converted to TSO Product. In these studies, addition of Proteinase K decreases the percentage of Full-Length product converted to TSO Product to approximately 4%. Addition of PEG to a reaction also containing Proteinase K increases the percentage of Full-Length product converted to TSO Product to approximately 7.5-8.0%, which is within a range sufficient for our systems.

### Example 4: Polyethylene Glycol Increases Yield of RNA Isolated from Fixed Cells

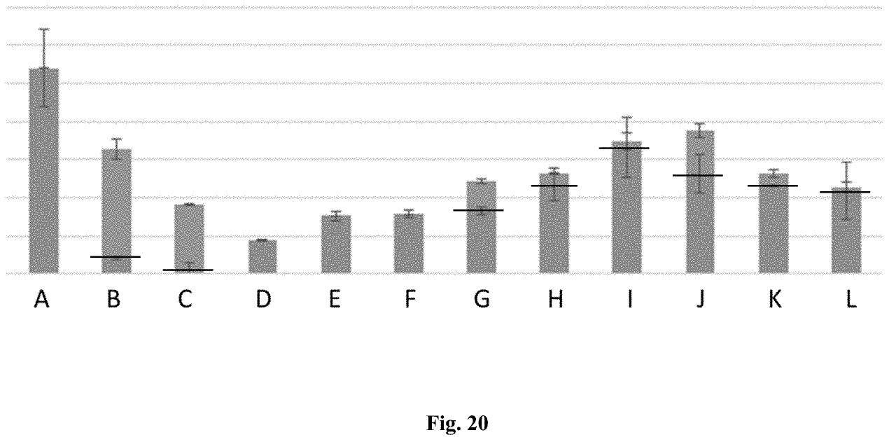

Since RT reactions, in the presence of un-fixing proteases, would be performed using template RNA from un-fixed cells, various conditions for maximizing recovery of RNA from fixed cells were tested. The experiments used Jurkat cells that were fixed in 4% paraformaldehyde at 4° C. for 24 hours. After 24 hours, the fixation reactions were quenched in 10% FBS-PBS. The fixed cells were then subjected to various un-fixing/de-crosslinking conditions (Proteinase K, 2-amino-5-methylbenzoic acid un-fixer and/or (4-aminopyridin-3-yl)phosphonic acid un-fixer) and then centrifuged. The amount of RNA present in both the centrifuged cell pellet and the supernatant from the centrifugation was determined. The data from these experiments are shown in the bar graph of FIG. 20. The height of the bar for each sample represents the sum of RNA from the pellet and supernatant. RNA was detected in the supernatant (“leakage” from the pellet) in samples B, C, G, H, I, J, K and L. For those samples, the amount of RNA in the supernatant is shown above the line drawn across each of those bars. In bars from samples in which no line across the bar is visible (samples D, E and F), the amount of RNA in the supernatant was negligible. All un-fixing reactions were performed at 53° C. for 45 min. When a single un-fixing agent was used in a reaction, its concentration was 50 mM. When two un-fixing agents were used in a reaction, the concentration of each was 25 mM. Additions to the reactions were as indicated below.

(A) RNA from fresh cells (positive control);

(B) 0.1 mg Proteinase K (PK) per ml;

(C) 0.1 mg PK per ml and 2-amino-5-methylbenzoic acid un-fixer;

(D) 0.1 mg PK per ml and (4-aminopyridin-3-yl)phosphonic acid un-fixer;

(E) 0.1 mg PK per ml, (4-aminopyridin-3-yl)phosphonic acid un-fixer and PEG 6000 (4% final concentration);

(F) 0.1 mg PK per ml, (4-aminopyridin-3-yl)phosphonic acid un-fixer and PEG 6000 (8% final concentration);

(G) 0.1 mg PK per ml, 2-amino-5-methylbenzoic acid and (4-aminopyridin-3-yl)phosphonic acid un-fixers;

(H) 0.1 mg PK per ml, 2-amino-5-methylbenzoic acid un-fixer, (4-aminopyridin-3-yl)phosphonic acid un-fixer and PEG 6000 (4% final concentration);

(I) 0.1 mg PK per ml, 2-amino-5-methylbenzoic acid un-fixer, (4-aminopyridin-3-yl)phosphonic acid un-fixer and PEG 6000 (8% final concentration);

(J) 0.2 mg PK per ml, 2-amino-5-methylbenzoic acid un-fixer and (4-aminopyridin-3-yl)phosphonic acid un-fixer;

(K) 0.2 mg PK per ml, 2-amino-5-methylbenzoic acid un-fixer, (4-aminopyridin-3-yl)phosphonic acid un-fixer and PEG 6000 (4% final concentration); and

(L) 0.2 mg PK per ml, 2-amino-5-methylbenzoic acid un-fixer, (4-aminopyridin-3-yl)phosphonic acid un-fixer and PEG 6000 (8% final concentration).

As shown in sample B, Proteinase K alone to un-fix cells was sufficient to yield 60% of the RNA (most of it in the supernatant) obtained from an equivalent number of fresh cells (compare with sample A). In other experiments, it had previously been shown that no RNA was obtained from un-fixed cells in absence of Proteinase K. Addition of the 2-amino-5-methylbenzoic acid un-fixer to Proteinase K decreased the total amount of recovered RNA, as well as the amount of RNA recovered in the pellet (sample C). Addition of the 4-aminopyridin-3-yl)phosphonic acid un-fixer to the Proteinase K increased the amount of RNA recovered in the pellet of centrifuged cells (compare sample D with samples B and C), although total amount of RNA recovered was less. Addition of PEG 6000 at either 4% (sample E) or 8% (sample F) to Proteinase K and 4-aminopyridin-3-yl)phosphonic acid un-fixer increased both the total amount of recovered RNA, as well as the amount of RNA recovered in the pellet (samples E and F) compared to Proteinase K and 4-aminopyridin-3-yl)phosphonic acid un-fixer alone.

Use of Proteinase K, 2-amino-5-methylbenzoic acid and 4-aminopyridin-3-yl)phosphonic acid un-fixers increased total RNA and RNA recovered in the cell pellet (sample G) compared to Proteinase K plus 2-amino-5-methylbenzoic acid (sample C), Proteinase K plus (4-aminopyridin-3-yl)phosphonic acid (sample D), and Proteinase K plus (4-aminopyridin-3-yl)phosphonic acid in the presence of PEG (samples E and F). Four percent final concentration of PEG 6000 to the un-fixing reaction containing Proteinase K, 2-amino-5-methylbenzoic acid and (4-aminopyridin-3-yl)phosphonic acid increased both total RNA and pellet RNA (sample H).

Eight percent final concentration of PEG 6000 in this situation increased both total and pellet RNA even more (sample I).

Increasing Proteinase K concentration from 0.1% (Sample G) to 0.2% (sample J) in presence of both 2-amino-5-methylbenzoic acid un-fixer and (4-aminopyridin-3-yl)phosphonic acid increased both total and pellet RNA recovery. But, at 0.2% Proteinase K, PEG 6000 did not increase the recovery as compared to absence of PEG (samples K and L).

Additional studies demonstrated that RNA obtained from both cell pellets and supernatants, as described above, served as templates for synthesis of cDNA using reverse transcriptase.

### Example 5: Use of Polyethylene Glycol (PEG) with Fixed Cells in Partitions (e.g., Droplets or Wells Containing Single Cells)

Polyethylene glycol is used in partitions within single-cell partitions (e.g., droplets or wells), along with fixed single cells and un-fixing agents, to (i) increase the yield and/or quality of mRNA from the single cells, and/or (ii) improve reverse transcriptase activity in the presence of an un-fixing agent (e.g., a protease).

In one example, a master mix that contains ingredients to be partitioned into droplets is formulated with increasing concentrations of PEG 600, PEG 1000, PEG 2000, PEG 6000 and/or PEG 10000 (e.g. 0.5%. 1%. 2% w/v). Un-fixing is performed in the partitions and, in one example, a single-cell 3′ protocol is performed. In an example, the single-cell 3′ protocol is performed in bulk without PEG (or without additional PEG). In an example, the single-cell 3′ protocol is performed in bulk in presence of PEG (or in presence of additional PEG).

Generally, the data generated from this experiment will show any improvements to the quality and/or yield of mRNA from cells that are un-fixed in presence of PEG in a droplet environment, as compared to libraries made from mRNA from cells un-fixed without PEG in droplets. The un-fixing may be performed in droplets using a protease (but optionally including a catalyst described herein, e.g., Compound 1 and/or Compound 8). The quality and/or yield of mRNA may be assessed based on the quality of nucleic acid libraries that are generated based on mRNA captured by in-droplet hybridization to nucleic acid barcode molecules (e.g., poly-T based hybridization) followed by bulk (i.e., outside of droplets) reverse transcription to generate cDNA molecules, which can be used to generate the nucleic acid library for sequencing.

In another example, the single cell 3′ protocol is performed with droplets containing PEG, a reverse transcriptase (RT), and a protease (but optionally including a catalyst described herein, e.g., Compound 1 and/or Compound 8). Generally, the data generated from this experiment will show any improvements to the quality and/or yield of mRNA from cells that are un-fixed in the presence of PEG in a droplet environment, as compared to libraries made from mRNA from cells un-fixed without PEG in droplets. The un-fixing may be performed in droplets using a protease (but optionally including a catalyst described herein, e.g., Compound 1 and/or Compound 8). The quality and/or yield of mRNA may be assessed based on the quality of nucleic acid libraries that are generated based on mRNA captured by in-droplet hybridization to nucleic acid barcode molecules (e.g., poly-T based hybridization) followed by in-droplet reverse transcription to generate cDNA molecules, which can be used to generate the nucleic acid library for sequencing.

1. A method for processing a nucleic acid molecule, comprising:
a) providing a partition comprising: (i) a fixed biological particle or a fixed membrane bound particle comprising a nucleic acid molecule comprising a nucleic acid sequence, (ii) a nucleic acid barcode molecule, and (iii) a cleaving agent;
b) subjecting said partition to a condition sufficient to generate a nucleic acid molecule comprising said nucleic acid sequence coupled to said nucleic acid barcode molecule;
c) releasing said nucleic acid molecule comprising said nucleic acid sequence coupled to said nucleic acid barcode molecule from said partition to generate a released nucleic acid molecule comprising said nucleic acid sequence coupled to said nucleic acid barcode molecule; and
d) subjecting said released nucleic acid molecule comprising said nucleic acid sequence coupled to said nucleic acid barcode molecule to a condition sufficient to extend said nucleic acid barcode molecule, using said nucleic acid sequence as a template and performed in presence of polyethylene glycol, to generate a barcoded nucleic acid molecule.

2. The method of claim 1, wherein said nucleic acid molecule is a cross-linked nucleic acid molecule, a cross-linked ribonucleic acid (RNA) molecule, or a cross-linked messenger RNA (mRNA) molecule.

3. The method of claim 2, wherein b) comprises: (i) generating an unlinked nucleic acid molecule from said cross-linked nucleic acid molecule and (ii) allowing said unlinked nucleic acid molecule to couple with said nucleic acid barcode molecule.

4. The method of claim 1, wherein said cleaving agent is a protease and, optionally, wherein d) is performed in presence of a protease inhibitor.

5. The method of claim 1, wherein the partition further comprises: (iv) a catalyst and/or (v) polyethylene glycol.

6. The method of claim 1, wherein the fixed biological particle or fixed membrane bound particle is a fixed single cell, a fixed single nucleus, a cell, a nucleus, or a virus.

7. (canceled)

8. (canceled)

9. The method of claim 1, wherein b) comprises heating said partition, and wherein the method further optionally comprises, subsequent to b) and prior to c), cooling said partition or allowing said partition to cool.

10. (canceled)

11. (canceled)

12. (canceled)

13. The method of claim 1, wherein d) comprises using a reverse transcriptase to extend said nucleic acid molecule barcode molecule, and optionally wherein said reverse transcriptase comprises RNase activity.

14. (canceled)

15. The method of claim 1, wherein said nucleic acid barcode molecule comprises a capture sequence configured to anneal to a nucleic acid molecule of said fixed biological particle or said fixed membrane bound particle.

16. The method of claim 1, further comprising:
appending an additional sequence to said barcoded nucleic acid molecule, optionally wherein said additional sequence is a poly-C sequence, and wherein said appending is performed by a ligase or a polymerase or a reverse transcriptase or by using a splint nucleic acid molecule; and
optionally subjecting said partition to a condition sufficient to hybridize a template switching oligonucleotide (TSO) to said additional sequence and extending said barcoded nucleic acid molecule to generate an extended barcoded nucleic acid molecule comprising a sequence complementary to said TSO.

17. (canceled)

18. (canceled)

19. (canceled)

20. (canceled)

21. (canceled)

22. (canceled)

23. (canceled)

24. The method of claim 1, wherein said nucleic acid barcode molecule is coupled to a support, optionally wherein said support is a bead or optionally wherein said nucleic acid barcode molecule is coupled to said support by a labile moiety.

25. (canceled)

26. (canceled)

27. The method of claim 1, wherein said partition is a droplet or a well.

28. (canceled)

29. (canceled)

30. The method of claim 1, further comprising: e) sequencing said barcoded nucleic acid molecule or an amplification product thereof.

31. The method of claim 1, further comprising:
providing a plurality of partitions
prior to a), partitioning a plurality of fixed biological particles or a plurality of fixed membrane bound particles into the plurality of partitions;
subsequent to b) and prior to c), said plurality of partitions comprise a plurality of nucleic acid molecules coupled to nucleic acid barcode molecules; and
subsequent to c), pooling said plurality of nucleic acid molecules coupled to said nucleic acid barcode molecules from said plurality of partitions.

32. (canceled)

33. (canceled)

34. (canceled)

35. The method of claim 1, further comprising prior to a), fixing a biological particle or a membrane bound particle to generate said fixed biological particle or said fixed membrane bound particle, wherein said fixing comprises use of a fixation agent, and optionally wherein said fixation agent comprises paraformaldehyde.

36. (canceled)

37. (canceled)

38. (canceled)

39. (canceled)

40. (canceled)

41. (canceled)

42. (canceled)

43. (canceled)

44. (canceled)

45. A method, comprising:
a) un-fixing or partially un-fixing fixed cells, nuclei or tissues using an un-fixing agent; and
b) synthesizing a complementary single-stranded DNA using a nucleic acid template from the un-fixed or partially un-fixed cells, nuclei or tissues and a reverse transcriptase (RT),
wherein a) and/or b) is conducted in presence of a polyethylene glycol (PEG).

46. The method of claim 45, wherein the un-fixing agent removes crosslinks formed by an aldehyde, including paraformaldehyde and/or glutaraldehyde; an NHS ester, including N-Hydroxysuccinimide; or an imidoester, that are present in biomolecules in the fixed cells or nuclei.

47. The method of claim 45, wherein the un-fixing agent has protease activity and/or wherein the un-fixing agent includes a catalyst.

48. (canceled)

49. (canceled)

50. (canceled)

51. (canceled)

52. The method of claim 45, wherein a) is performed in a partition.

53. The method of claim 45, further, comprising: isolating a nucleic acid from the un-fixed cells or nuclei.

54. (canceled)

55. (canceled)

56. (canceled)

57. (canceled)

58. (canceled)

59. (canceled)

60. (canceled)

61. (canceled)

62. (canceled)


SEQUENCE LISTING (3 sequences)

SEQ ID NO: 1 - DNA, 18 bp, synthetic construct
  source 1..18: mol_type = other DNA
cgactcactgacactcgc

SEQ ID NO: 2 - RNA, 82 nt, synthetic construct
  source 1..82: mol_type = other RNA
acgaccgtcgtcatgtagcgtttgtcggagactcctagatcagatgtcctcctggctactgcacgcgagtgtcagtgagtcg

SEQ ID NO: 3 - DNA, 30 bp, synthetic construct
  source 1..30: mol_type = other DNA
aagcagtggtatcaacgcagagtacatggg